SEQUENCE LISTING (14 sequences)

SEQ ID NO: 1 - DNA, 29 bp, Artificial Sequence
  Consensus sequence
acaggcgattcgtcagtccgctgtagcgt

SEQ ID NO: 2 - DNA, 29 bp, Artificial Sequence
  Consensus sequence
acaggcgattcgtcagtccgctgtagcgt

SEQ ID NO: 3 - DNA, 29 bp, Artificial Sequence
  Contig Consensus sequence
acaggcgattcgtcagtccgctgtagcgt

SEQ ID NO: 4 - DNA, 12 bp, Artificial Sequence
  Consensus sequence
cagcagcagcag

SEQ ID NO: 5 - DNA, 21 bp, Artificial Sequence
  Consensus sequence
cagcagcagcagcagcagcag

SEQ ID NO: 6 - DNA, 21 bp, Artificial Sequence
  Consensus sequence
cagcagcagcagcagcagcag

SEQ ID NO: 7 - DNA, 12 bp, Artificial Sequence
  Consensus sequence
cagcagcagcag

SEQ ID NO: 8 - DNA, 21 bp, Artificial Sequence
  Contig Consensus sequence
cagcagcagcagcagcagcag

SEQ ID NO: 9 - DNA, 15 bp, Artificial Sequence
  Flanking sequence
cagcagcagcagcag

SEQ ID NO: 10 - DNA, 29 bp, Artificial Sequence
  Consensus sequence
acaggcgattcgtcagtccgctgtagcgt

SEQ ID NO: 11 - protein, 21 aa, Homo sapiens
TLVVSSIDHISSDFKIMLKET

SEQ ID NO: 12 - protein, 19 aa, Homo sapiens
PWWFLPLTTSPLTSKSCKK

SEQ ID NO: 13 - protein, 18 aa, Homo sapiens
LGGFFHPHLLLQNHAERN

SEQ ID NO: 14 - protein, 17 aa, Homo sapiens
GQHNRGNVVDGRVEFDH

(19) United States
(12) Patent Application Publication
(10) Pub. No.: US 2019/0244678 A1
(43) Pub. Date: Aug. 8, 2019

(54) METHODS, SYSTEMS AND PROCESSES OF DE NOVO ASSEMBLY OF SEQUENCING READS

(71) Applicant: INVITAE CORPORATION, San Francisco, CA (US)

(72) Inventors: Karel KONVICKA, San Francisco, CA (US); Kevin JACOBS, San Francisco, CA (US)

(21) Appl. No.: 15/513,374

(22) PCT Filed: Oct. 9, 2015
(86) PCT No.: PCT/IB15/57716

§ 371 Date: Mar. 22, 2017

Related U.S. Application Data
(60) Provisional application No. 62/062,636, filed on Oct. 10, 2014.

(51) Int. Cl. G16B 20/20; G16B 30/00; G16B 20/00

(52) U.S. Cl. CPC G16B 20/20; G16B 20/00; G16B 30/00

(57) ABSTRACT

Provided herein are novel methods, systems and processes of mapping and assembling sequence reads. Also provided herein are methods, systems and processes of identifying the presence or absence of a genetic variation in a genome of a subject.

### RELATED PATENT APPLICATIONS

This patent application claims the benefit of Provisional Patent Application No. 62/062,636 filed on Oct. 10, 2014, entitled “METHODS, SYSTEMS AND PROCESSES OF DE NOVO ASSEMBLY OF SEQUENCING READS”, naming Karel Konvicka and Kevin Jacobs as an inventor, and designated by attorney docket no. 055911-0432229. The entire content of the foregoing patent application is incorporated herein by reference, including all text, tables and drawings.

### FIELD

The technology relates in part to methods and processes of nucleic acid manipulation, analysis and high-throughput sequencing.

### BACKGROUND

Genetic information of living organisms (e.g., animals, plants, microorganisms, viruses) is encoded in deoxyribonucleic acid (DNA) or ribonucleic acid (RNA). Genetic information is a succession of nucleotides or modified nucleotides representing the primary structure of nucleic acids. The nucleic acid content (e.g., DNA) of an organism is often referred to as a genome. In humans, the complete genome typically contains about 30,000 genes located on twenty-four (24) chromosomes. Most genes encode a specific protein, which after expression via transcription and translation fulfills one or more biochemical functions within a living cell.

Many medical conditions are caused by one or more genetic variations within a genome. Some genetic variations may predispose an individual to, or cause, any of a number of diseases such as, for example, diabetes, arteriosclerosis, obesity, various autoimmune diseases and cancer (e.g., colorectal, breast, ovarian, lung). Such genetic diseases can result from an addition, substitution, insertion or deletion of one or more nucleotides within a genome.

Genetic variations can be identified by analysis of nucleic acids. Nucleic acids of a genome can be analyzed by various methods including, for example, methods that involve massively parallel sequencing. Massively parallel sequencing techniques often generate thousands, millions or even billions of small sequencing reads. To determine genomic sequences, each read is often mapped to a reference genome and collections of reads are assembled, into a sequence representation of an individual's genome, or portions thereof. The process of mapping and assembly of reads is carried out by one or more computers (e.g., hardware microprocessors (i.e., microprocessors) and memory) and is driven by a set of instructions (e.g., software instructions and/or algorithms) created by the hand of man. Such mapping and assembly processes often fail when a genetic variation is encountered in a genome of a subject. Existing software and programs incorrectly map reads, fail to map reads and fail to correctly assemble regions of a genome that comprise a genetic variation. Methods, systems and processes herein offer significant advances and improvements to current nucleic acid analysis techniques.

### SUMMARY

In some aspects provided herein is a method of analyzing a nucleic acid library comprising a non-transitory computer-readable storage medium with an executable program stored thereon, which program is configured to instruct a microprocessor to (a) obtain a set of paired-end sequence reads comprising a plurality of read mate pairs, each pair comprising two read mates, where at least one of the two read mates of each pair is mapped to at least one portion of a reference genome comprising a pre-selected genomic region of interest and where some of the paired-end sequence reads were not mapped to the at least one portion of the reference genome, (b) determine a pile-up relationship for the set of sequence reads, (c) construct one or more contigs according to the pile-up relationship determined in (b), comprising iteratively adding at least one nucleotide to a position 3′ or 5′ of one or more starter reads where the position (e.g., the advancing position) includes a majority consensus nucleotide, (d) assemble one or more supercontigs, according to one or more read mate pairs that bridge two or more contigs, (e) generate a genotype likelihood ratio according to the one or more supercontigs, and (f) determine the presence or absence of genetic alteration according to the genotype likelihood ratio generated in (e).

In some aspects the pile-up relationship comprises a plurality of overlaps between two or more reads of the set where each of the plurality of overlaps is selected according to (i) a first read of the set that comprises a first overlap with a second read of the set, (ii) the first overlap includes an alignment score that is greater than a predetermined alignment score threshold, (iii) the second read extends one or more nucleotides past a 3′ end or a 5′ end of the first read, and (iv) the first overlap includes a highest alignment score of all possible first overlaps that satisfies (i), (ii) and (iii). In some aspects the pile-up relationship comprises a second read comprising a second overlap with a third read of the set, where (i) the second read includes the first overlap, (ii) the second overlap includes an alignment score that is greater than a predetermined alignment score threshold, (iii) the third read extends one or more nucleotides past a 3′ end or a 5′ end of the second read, and the second read and the third read extend the first read in a same 3′ or 5′ direction, and (iv) the second overlap includes the highest alignment score of all possible second overlaps that satisfy (i), (ii) and (iii).

In some aspects a majority consensus nucleotide is determined according to the plurality of overlaps determined for the pile-up relationship. In certain embodiments, constructing the contig comprises iteratively adding at least one nucleotide to a position 3′ or 5′ of each of one or more intermediate contigs. In some embodiments, where the position (e.g., the advancing position) comprises two different majority consensus nucleotides, constructing the contig comprises generating a copy of the intermediate contig, thereby providing two identical intermediate contigs, adding one of the two different majority consensus nucleotides to each of the two identical intermediate contigs, where a different nucleotide is added to each of the two identical intermediate contigs. In some embodiments, where the position (e.g., the advancing position) comprises three different majority consensus nucleotides, constructing the contig comprises generating two copies of the intermediate contig, thereby providing three identical intermediate contigs, adding one of the three different majority consensus nucleotides to each of the three identical intermediate contigs, where a different nucleotide is added to each of the three identical intermediate contigs. In some embodiments, where the position (e.g., the advancing position) comprises four different majority consensus nucleotides, constructing the contig comprises generating three copies of the intermediate contig, thereby providing four identical intermediate contigs, adding one of the four different majority consensus nucleotides to each of the four identical intermediate contigs, where a different nucleotide is added to each of the four identical intermediate contigs.

In some aspects samples are obtained from one or more human subjects.

Certain embodiments are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 shows an embodiment of a system flow chart (e.g., Kragle).

FIG. 2 shows an embodiment of an overlap and an example of read-read connection filtering with the default minimum count of extending reads (set to 1). A read (red color) has reads A through G extending it to the right (green color). Some of these extending reads have also reads extending them to the right (blue color). The red read will keep 3 connections to reads extending it to the right. It will keep read A because it has the highest score, but since read A does not have any read extending it to the right, the red read will also keep read B and C. These two reads have the same score (1200) and do have reads extending them to the right.

No additional read connections are necessary; the red read has among the three connections at least one read that itself can be extended to the right (both reads B and C can be extended to the right by another read).

FIG. 3 shows and embodiment of an overlap. Read A has to keep connections to both read B (on haplotype with polymorphic base A) and read C (on haplotype with polymorphic base C). It will keep the connection to read B because it is the best scoring read extending read A to the right (and if read B has itself a read extending it to the right, then read A does not need itself additional connections). However read A is the best scoring read extending read C to the left, therefore the connection between read A and read C is forced by read C. Thus read A will have two connections, both extending it to the right, however each one to a different haplotype.

FIG. 4 shows an embodiment of a contig assembly showing an extension of “all recruited” edge and the consensus sequence. All reads that are inside the one-base extended “all recruited” interval will recruit in the current iteration.

FIG. 5 shows an embodiment of a contig assembly where more than one majority consensus nucleotide exists for the advancing position. FIG. 5 shows a representation of splitting (copying) of contigs. An A/C polymorphic position is encountered and causes the current contig to be split into two. From the five reads with base A (color blue) three reads (or their mates) are crossing the previous split position. From the three read pairs with base C (color green) 2 cross the previous split position. No base gets haplotype adjusted count 0, therefore two new contigs will be generated from the current contig; in addition to the red reads one contig will take the read pairs with base A (blue reads) and the other contig will take the read pairs with base C (green reads).

FIG. 6 shows a polymorphic read pileup that does not support contig splitting because the reads with polymorphic base C have haplotype-adjusted count 0 (e.g., no read pair with base C crosses the previous split position). Therefore the read pairs with base C will be eliminated from the contig and will not generate a new contig.

FIG. 7 shows an embodiment of a supercontigs assembly. The red contig in a) encounters first A/C polymorphic position and splits off new contig (blue). The red contig encounters another G/T polymorphic position and splits off another contig (green). When the blue contig is built it encounters G/T polymorphic position that is identical (identical read composition and the positions in the reads) to the G/T split of the red contig. This split is therefore only marked and the remainders of the blue contig are not constructed. However, during contig consolidation phase, the blue contig is appended with the two possible endings of the red contig that had the matching split, resulting in the first two of the four contigs in b).

FIG. 8 shows an embodiment of how contigs are connected to form supercontigs. The following graph of contig connections results in 5 supercontigs (5 paths through the graph from the start nodes to the end nodes).

FIG. 9 shows read-pair alignments to hypothetical sequence around false insertion. False insertion is composed of reads (blue), which mates do not cross over to the neighboring flanks. Such false insertions usually occur in repetitive regions where some false alignments can be accomplished between the reads from the true repeat and the repeat sequence in reads originating from sequence outside of this region.

FIG. 10 shows an overview of an embodiment comprising forming pile-up relationships, assembling contigs, assembling supercontigs and generating genotype probabilities.

FIG. 11 describes an example of a process of generating pile-up relationships (e.g. read-read alignments).

FIG. 12 shows an embodiment of filtering overlaps.

FIG. 13 shows an embodiment of read alignment graph cycles.

FIG. 14 shows an embodiment of assembling contigs and/or supercontigs.

FIG. 15 describes an embodiment of contig assembly.

FIG. 16 shows another embodiment of contig assembly.

FIG. 17 describes an embodiment comprising spitting (e.g., copying) contigs during a contig assembly process.

FIG. 18 describes an embodiment of finalizing a contig assembly.

FIG. 19 shows an embodiment of assembling supercontigs.

FIG. 20 shows an example of diploid hypothesis.

FIG. 21 show an example of a genotype likelihood model that includes an insertion penalty portion.

FIG. 22 shows an example of a derivation of separate allele representation.

FIG. 23 shows an embodiment of a portion of a Kragle methodology.

FIG. 24 shows an example of the results obtained by applying Kragle.

FIG. 25 shows an example of a CFTR caller.

FIG. 26 show an example of a challenging assembly task.

FIG. 27 shows an example of mapping of two assembled haplotypes for the confirmed heterozygous deletion in exon 19 of BRCA1 gene. The figure displays the mapping of the 3′ side of haplotype 2 that contains the deletion.

FIG. 28 shows an example of mapping of the 5′ side of haplotype 2 and assembling an experimentally confirmed deletion in exon 19 of BRCA1 gene.

### DETAILED DESCRIPTION

Next generation sequencing (NGS) allows for sequencing nucleic acids on a genome-wide scale by methods that are faster and cheaper than traditional methods of sequencing. Methods and processes herein provide for improvements of advanced sequencing technologies that can be used to locate and identify genetic variations and/or associated diseases and disorders. In some embodiments, provided herein are methods that comprise, in part, manipulation and analysis of sequence reads that are often obtained by a massively parallel sequencing method.

Traditional assemblers and aligners often fail to correctly assemble genomic sequences that contain genetic variation (e.g., short tandem repeats (STRs), polymorphisms, insertions, etc.). Calling genetic variation such as STRs is a difficult problem for most aligners and mappers. Existing algorithms and software packages fail to correctly map and align reads in genomic regions that comprise such genomic variations. Examples of assemblers that were tested and failed in this regard include Lobstr, Repeatseq and general de-novo assemblers such as GATK's Haplotype Caller, AMOS de-novo assembler, Mira de-novo assembler, FERMI, SGA and others. There is a great need for new and improved systems and methods (e.g., microprocessor dependent methods) that can correctly and routinely assemble genomic regions that comprise genetic variations and/or accurately identify genetic variation from a set of sequencing reads. Such methods, systems and processes are described and claimed herein.

Subjects

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. A subject can be a patient (e.g. a human patient).

Samples

Provided herein are methods and compositions for analyzing a sample. A sample (e.g., a sample comprising nucleic acid) can be obtained from a suitable subject. A sample can be isolated or obtained directly from a subject or part thereof. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-free). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may comprise cells or tissues that are normal, healthy, diseased (e.g., infected), and/or cancerous (e.g., cancer cells). A sample obtained from a subject may comprise cells or cellular material (e.g., nucleic acids) of multiple organisms (e.g., virus nucleic acid, fetal nucleic acid, bacterial nucleic acid, parasite nucleic acid).

In some embodiments, a sample comprises nucleic acid, or fragments thereof. A sample can comprise nucleic acids obtained from one or more a subjects. In some embodiments a sample comprises nucleic acid obtained from a single subject. In some embodiments, a sample comprises a mixture of nucleic acids. A mixture of nucleic acids can comprise two or more nucleic acid species having different nucleotide sequences, different fragment lengths, different origins (e.g., genomic origins, cell or tissue origins, subject origins, the like or combinations thereof), or combinations thereof. A sample may comprise synthetic nucleic acid.

Nucleic Acids

The terms “nucleic acid” refers to one or more nucleic acids (e.g., a set or subset of nucleic acids) of any composition from, such as DNA (e.g., complementary DNA (cDNA), genomic DNA (gDNA) and the like), RNA (e.g., message RNA (mRNA), short inhibitory RNA (siRNA), ribosomal RNA (rRNA), tRNA, microRNA, and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. Unless specifically limited, the term encompasses nucleic acids comprising deoxyribonucleotides, ribonucleotides and known analogs of natural nucleotides. A nucleic acid may include, as equivalents, derivatives, or variants thereof, suitable analogs of RNA or DNA synthesized from nucleotide analogs, single-stranded (“sense” or “antisense”, “plus” strand or “minus” strand, “forward” reading frame or “reverse” reading frame) and double-stranded polynucleotides. Nucleic acids may be single or double stranded. A nucleic acid can be of any length of 2 or more, 3 or more, 4 or more or 5 or more contiguous nucleotides. A nucleic acid can comprise a specific 5′ to 3′ order of nucleotides known in the art as a sequence (e.g., a nucleic acid sequence, e.g., a sequence).

A nucleic acid may be naturally occurring and/or may be synthesized, copied or altered by the hand of man. For, example, a nucleic acid may be an amplicon. A nucleic acid may be from a nucleic acid library, such as a gDNA, cDNA or RNA library, for example. A nucleic acid can be synthesized (e.g., chemically synthesized) or generated (e.g., by polymerase extension in vitro, e.g., by amplification, e.g., by PCR). A nucleic acid may be, or may be from, a plasmid, phage, virus, autonomously replicating sequence (ARS), centromere, artificial chromosome, chromosome, or other nucleic acid able to replicate or be replicated in vitro or in a host cell, a cell, a cell nucleus or cytoplasm of a cell in certain embodiments. Nucleic acids (e.g., a library of nucleic acids) may comprise nucleic acid from one sample or from two or more samples (e.g., from 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more samples). Nucleic acid provided for processes or methods described herein may comprise nucleic acids from 1 to 1000, 1 to 500, 1 to 200, 1 to 100, 1 to 50, 1 to 20 or 1 to 10 samples.

The term “gene” means the segment of DNA involved in producing a polypeptide chain and can include regions preceding and following the coding region (leader and trailer) involved in the transcription/translation of the gene product and the regulation of the transcription/translation, as well as intervening sequences (introns) between individual coding segments (exons). A gene may not necessarily produce a peptide or may produce a truncated or non-functional protein due to genetic variation in a gene sequence (e.g., mutations in coding and non-coding portions of a gene). A gene, whether functional or non-functional, can often be identified by homology to a gene in a reference genome.

Oligonucleotides are relatively short nucleic acids. Oligonucleotides can be from about 2 to 150, 2 to 100, 2 to 50, or 2 to about 35 nucleic acids in length. In some embodiments oligonucleotides are single stranded. In certain embodiments, oligonucleotides are primers. Primers are often configured to hybridize to a selected complementary nucleic acid and are configured to be extended by a polymerase after hybridizing.

Nucleic Acid Isolation & Purification

Nucleic acid may be derived, isolated, extracted, purified or partially purified from one or more subjects, one or more samples or one or more sources using suitable methods known in the art. Any suitable method can be used for isolating, extracting and/or purifying nucleic acid.

The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a subject (e.g., a human subject). An isolated nucleic acid can be provided with fewer non-nucleic acid molecules (e.g., protein, lipid, small compounds, carbohydrate, contaminants, particles, aggregates, salts, detergents, etc.) than an amount of non-nucleic acid molecules present in a source sample. A composition comprising isolated nucleic acid can be about 50% to greater than 99% free of non-nucleic acid molecules. A composition comprising isolated nucleic acid can be about 90%, 91%, 92%, 93%, 94%, 95%, 96%, 97%, 98%, 99% or greater than 99% free of non-nucleic acid molecules. The term “purified” as used herein can refer to a nucleic acid provided that contains fewer non-nucleic acid molecules than the amount of non-nucleic acid molecules present prior to subjecting the nucleic acid to a purification procedure. A composition comprising purified nucleic acid may be at least about 60%, 70%, 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid molecules. A composition comprising purified nucleic acid may be at least about 60%, 70%, 80%, 81%, 82%, 83%, 84%, 85%, 86%, 87%, 88%, 89%, 90%, 91%, 92%, 93%, 94%, 95%, 96%, 97%, 98%, 99% or greater than 99% free of other nucleic acids. A composition comprising purified nucleic acid may comprise at least 80%, 81%, 82%, 83%, 84%, 85%, 86%, 87%, 88%, 89%, 90%, 91%, 92%, 93%, 94%, 95%, 96%, 97%, 98%, 99% or greater than 99% of the total nucleic acid present in a sample prior to application of a purification method.

Nucleic Acid Sequencing

In certain embodiments nucleic acids (e.g., amplicons, nucleic acids of a library, captured nucleic acids) are analyzed by a process comprising nucleic acid sequencing. In some embodiments, nucleic acids may be sequenced. In some embodiments, a full or substantially full sequence is obtained and sometimes a partial sequence is obtained.

A suitable method of sequencing nucleic acids can be used, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first generation technology, such as, for example, Sanger sequencing methods including automated Sanger sequencing methods, including microfluidic Sanger sequencing, can be used in a method provided herein. In some embodiments sequencing technologies that include the use of nucleic acid imaging technologies (e.g. transmission electron microscopy (TEM) and atomic force microscopy (AFM)), can be used. In some embodiments, a high-throughput sequencing method is used. High-throughput sequencing methods generally involve clonally amplified DNA templates or single DNA molecules that are sequenced in a massively parallel fashion, sometimes within a flow cell. Next generation (e.g., 2nd and 3rd generation) sequencing techniques capable of sequencing DNA in a massively parallel fashion can be used for methods described herein and are collectively referred to herein as “massively parallel sequencing” (MPS). Any suitable MPS or next generation sequencing method, system or technology platform for conducting methods described herein can be used to obtain sequencing reads, non-limiting examples of which include Illumina/Solex/HiSeq (e.g., Illumina's Genome Analyzer; Genome Analyzer II; HISEQ 2000; HISEQ 2500, SOLiD, Roche/454, PACBIO, SMRT, Helicos True Single Molecule Sequencing, Ion Torrent and Ion semiconductor-based sequencing, WildFire, 5500, 5500xl W and/or 5500xl W Genetic Analyzer based technologies (e.g., as developed and sold by Life Technologies), Polony sequencing; Pyrosequencing, Massively Parallel Signature Sequencing, RNA polymerase (RNAP) sequencing, IBS methods, LaserGen systems and methods, chemical-sensitive field effect transistor (CHEMFET) array, electron microscopy-based sequencing, nanoball sequencing, sequencing-by-synthesis, sequencing by ligation, sequencing-by-hybridization, the like or variations thereof. Additional sequencing technologies that include the use of developing nucleic acid imaging technologies (e.g. transmission electron microscopy (TEM) and atomic force microscopy (AFM)), also are contemplated herein. In some embodiments, a high-throughput sequencing method is used. High-throughput sequencing methods generally involve clonally amplified DNA templates or single DNA molecules that are sequenced in a massively parallel fashion, sometimes within a flow cell. In some embodiments MPS sequencing methods utilize a targeted approach, where sequence reads are generated from specific chromosomes, genes or regions of interest. Specific chromosomes, genes or regions of interest are sometimes referred to herein as targeted genomic regions. In certain embodiments a non-targeted approach is used where most or all nucleic acid fragments in a sample are sequenced, amplified and/or captured randomly.

Sequence Reads

Subjecting a nucleic acid to a sequencing method often provides sequence reads. As used herein, “reads” (e.g., “a read”, “a sequence read”) are short nucleotide sequences produced by any sequencing process described herein or known in the art. Reads can be generated from one end of a nucleic acid fragment (“single-end reads”), and sometimes are generated from both ends of a nucleic acid fragment (e.g., paired-end reads, paired-end sequence reads, double-end reads). Paired end reads often include one or more pairs of reads (e.g., two reads, a read mate pair) were each pair of reads is obtained from each end of a nucleic acid fragment that was sequenced. Each read of a read mate pair is sometimes referred to herein as a read mate. A paired end sequencing approach (e.g., where one or more libraries of nucleic acids are sequenced) often results in a plurality of read mate pairs and a plurality of read mates.

The length of a sequence read is often associated with the particular sequencing technology. High-throughput methods and/or next generation sequence, for example, provide sequence reads that can vary in size from tens to hundreds of base pairs (bp). In some embodiments, sequence reads are of a mean, median, average or absolute length of about 15 bp to about 900 bp long. In certain embodiments sequence reads are of a mean, median, average or absolute length about 1000 bp or more.

Single end reads can be of any suitable length. In some embodiments the nominal, average, mean or absolute length of single-end reads sometimes is about 10 nucleotides to about 1000 contiguous nucleotides, about 10 nucleotide to about 500 contiguous nucleotides, about 10 nucleotide to about 250 contiguous nucleotides, about 10 nucleotide to about 200 contiguous nucleotides, about 10 nucleotide to about 150 contiguous nucleotides, about 15 contiguous nucleotides to about 100 contiguous nucleotides, about 20 contiguous nucleotides to about 75 contiguous nucleotides, or about 30 contiguous nucleotides or about 50 contiguous nucleotides. In certain embodiments the nominal, average, mean or absolute length of single-end reads is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 or more nucleotides in length.

Paired-end reads (e.g., read mates) can be of any suitable length. In certain embodiments, both ends of a nucleic acid fragment are sequenced at a suitable read length that is sufficient to map each read (e.g., reads of both ends of a fragment template) to a reference genome. In certain embodiments, the nominal, average, mean or absolute length of paired-end reads is about 10 contiguous nucleotides to about 500 contiguous nucleotides, about 10 contiguous nucleotides to about 400 contiguous nucleotides, about 10 contiguous nucleotides to about 300 contiguous nucleotides, about 50 contiguous nucleotides to about 200 contiguous nucleotides, about 100 contiguous nucleotides to about 200 contiguous nucleotides, or about 100 contiguous nucleotides to about 150 contiguous nucleotides. In certain embodiments, the nominal, average, mean or absolute length of paired-end reads is about 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170 or more nucleotides.

Reads generally are representations of nucleotide sequences in a physical nucleic acid. For example, in a read containing an ATGC depiction of a sequence, “A” represents an adenine nucleotide, “T” represents a thymine nucleotide, “G” represents a guanine nucleotide and “C” represents a cytosine nucleotide, in a physical nucleic acid. A mixture of relatively short reads can be transformed by processes described herein into a representation of a genomic nucleic acid present in subject. A mixture of relatively short reads can be transformed into a representation of a copy number variation (e.g., a copy number variation), genetic variation or an aneuploidy, for example. Reads of a mixture of nucleic acids from multiples subjects can be transformed into a representation of genome, or portion thereof, for each of the multiple subjects. In certain embodiments, “obtaining” nucleic acid sequence reads of a sample from a subject and/or “obtaining” nucleic acid sequence reads of a biological specimen from one or more reference persons can involve directly sequencing nucleic acid to obtain the sequence information. In some embodiments, “obtaining” can involve receiving sequence information obtained directly from a nucleic acid by another.

Mapping Reads

Sequence reads can be mapped. In some embodiments a suitable mapping method, process or algorithm can be used. In certain embodiments modified mapping methods and processes are used herein. Certain aspects of mapping processes are described hereafter.

Mapping nucleotide sequence reads (e.g., sequence information from a fragment whose physical genomic position is unknown) can be performed in a number of ways, and often comprises alignment of the obtained sequence reads, or portions thereof, with a matching sequence in a reference genome. In such alignments, sequence reads generally are aligned to a reference sequence and those that align are designated as being “mapped”, “a mapped sequence read” or “a mapped read”.

As used herein, the terms “aligned”, “alignment”, or “aligning” refer to two or more nucleic acid sequences that can be identified as a match (e.g., 100% identity) or partial match. Alignments can be done manually or by a computer (e.g., a software, program, computer program component, or algorithm), non-limiting examples of which include the Efficient Local Alignment of Nucleotide Data (ELAND) computer program distributed as part of the Illumina Genomics Analysis pipeline. Alignment of a sequence read can be a 100% sequence match. In some cases, an alignment is less than a 100% sequence match (e.g., non-perfect match, partial match, partial alignment). In some embodiments an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76% or 75% match. In some embodiments, an alignment comprises a mismatch. In some embodiments, an alignment comprises 1, 2, 3, 4 5 or more mismatches. Two or more sequences can be aligned using either strand. In certain embodiments a nucleic acid sequence is aligned with the reverse complement of another nucleic acid sequence.

Various computational methods can be used to map and/or align sequence reads to a reference genome. Sequence reads can be mapped by a mapping component or by a machine or computer comprising a mapping component (e.g., a suitable mapping and/or alignment program), which mapping component generally maps reads to a reference genome or segment thereof. Sequence reads and/or paired-end reads are often mapped to a reference genome by use of a suitable mapping and/or alignment program non-limiting examples of which include BWA (Li H. and Durbin R. (2009)Bioinformatics 25, 1754-60), Novoalign [Novocraft (2010)], Bowtie (Langmead B, et al., (2009) Genome Biol. 10:R25), SOAP2 (Li R, et al., (2009) Bioinformatics 25, 1966-67), BFAST (Homer N, et al., (2009) PLoS ONE 4, e7767), GASSST (Rizk, G. and Lavenier, D. (2010) Bioinformatics 26, 2534-2540), and MPscan (Rivals E., et al. (2009) Lecture Notes in Computer Science 5724, 246-260), or the like. Sequence reads and/or paired-end reads can be mapped and/or aligned using a suitable short read alignment program. Non-limiting examples of short read alignment programs are BarraCUDA, BFAST, BLASTN, BLAST, BLAT, BLITZ, Bowtie (e.g., BOWTIE 1, BOWTIE 2), BWA, CASHX, CUDA-EC, CUSHAW, CUSHAW2, drFAST, FASTA, ELAND, ERNE, GNUMAP, GEM, GensearchNGS, GMAP, Geneious Assembler, iSAAC, LAST, MAQ, mrFAST, mrsFAST, MOSAIK, MPscan, Novoalign, NovoalignCS, Novocraft, NextGENe, Omixon, PALMapper, Partek, PASS, PerM, PROBEMATCH, QPalma, RazerS, REAL, cREAL, RMAP, rNA, RTG, Segemehl, SeqMap, Shrec, SHRiMP, SLIDER, SOAP, SOAP2, SOAPS, SOCS, SSAHA, SSAHA2, Stampy, SToRM, Subread, Subjunc, Taipan, UGENE, VelociMapper, TimeLogic, XpressAlign, ZOOM, the like, variations thereof or combinations thereof. A mapping component can map sequencing reads by a suitable method known in the art or described herein. In some embodiments, a mapping component or a machine or computer comprising a mapping component is required to provide mapped sequence reads. A mapping component often comprises a suitable mapping and/or alignment program or algorithm.

In some embodiments one or more sequence reads and/or information associated with a sequence read are stored on and/or accessed from a non-transitory computer-readable storage medium in a suitable computer-readable format. Information stored on a non-transitory computer-readable storage medium is sometimes referred to as a file or data file. Reads (e.g., individual reads, paired end reads, read mates, read mate pairs), selected reads, sets or subsets of reads and/or information associated with one or more reads is often stored in a file or data file. A file often comprises a format. For example, a sequence read is sometimes stored in a format that includes information about one or more sequence reads, non-limiting examples of such information includes a complete or partial nucleic acid sequence, mappability, a mappability score, a mapped location, a relative location or distance from other mapped or unmapped reads (e.g., estimated distance between read mates), orientation relative to a reference genome or to other reads (e.g., relative to read mates), an estimated or precise location of a read mates, G/C content, the like or combinations thereof. A “computer-readable format” is sometimes referred to generally herein as a format. In some embodiments sequence reads are stored and/or accessed in a suitable binary format, a text format, the like or a combination thereof. A binary format is sometimes a BAM format. A text format is sometimes a sequence alignment/map (SAM) format. Non-limiting examples of binary and/or text formats include BAM, sorted BAM, SAM, SRF, FASTA, FASTQ, Gzip, the like, or combinations thereof.

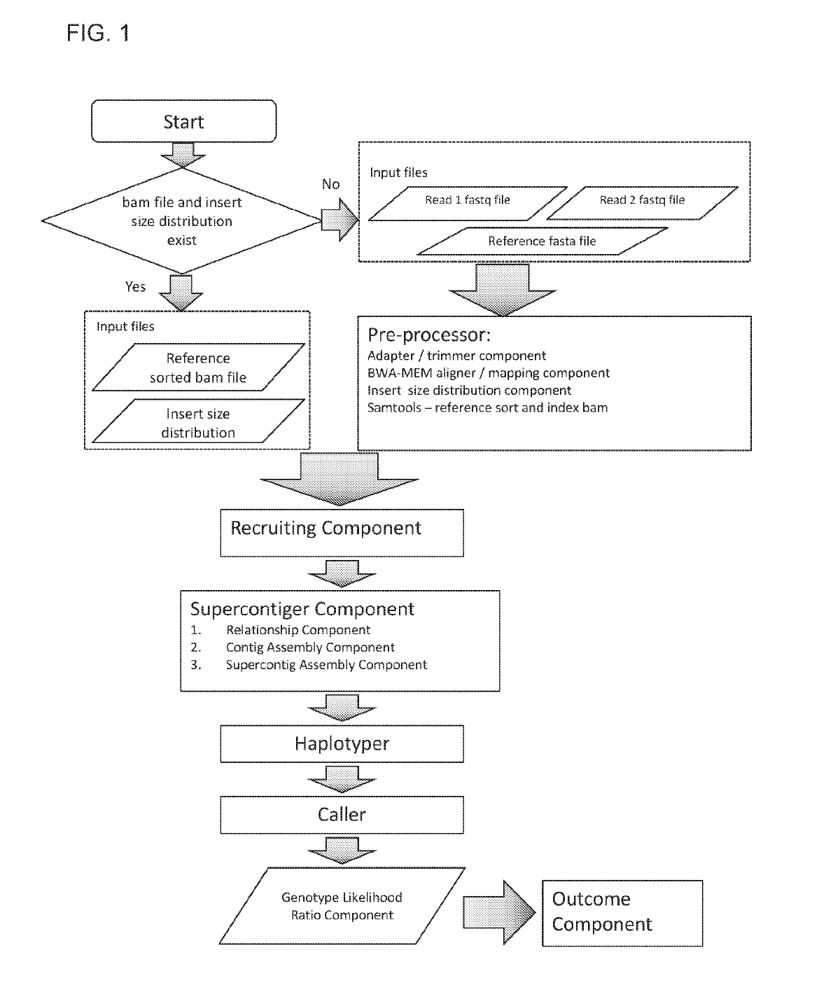

In some embodiments a program herein is configured to instruct a microprocessor to obtain or retrieve one or more files (e.g., sorted bam files). In some embodiments a program herein is configured to instruct a microprocessor to obtain or retrieve one or more FASTQ files (e.g., a FASTQ file for a first read and a second read) and/or one or more reference files (e.g., a FASTA or FASTQ file). In some embodiments a program herein instructs a microprocessor to call a computer program component and/or transfers data and/or information (e.g., files) to or from one or more computer program components (e.g., an adapter trimmer component, BWA-MEM aligner, insert size distribution component, samtools, and the like). In some embodiments a program instructs a processor to call a computer program component which creates new files and formats for input into another processing step (see Example 1 and FIG. 1). In some embodiments sequence reads in a first format are compressed into a second format requiring less storage space than the first format. The term “compressed” as used herein refers to a process of data compression, source coding, and/or bit-rate reduction where a computer readable data file is reduced in size. Non-limiting examples of a compression component include GZIP and BGZF, the like or modifications thereof.

In some embodiments, a read may uniquely or non-uniquely map to a reference genome. A read is considered as “uniquely mapped” if it aligns with a single sequence in the reference genome. A read is considered as “non-uniquely mapped” if it aligns with two or more sequences in a reference genome. In some embodiments, non-uniquely mapped reads are eliminated from further analysis (e.g., quantification). A certain, small degree of mismatch (0-1) may be allowed to account for single nucleotide polymorphisms that may exist between the reference genome and the reads from individual samples being mapped, in certain embodiments. In some embodiments, no degree of mismatch is allowed for a read mapped to a reference sequence.

As used herein, the term “reference genome” can refer to any particular known, sequenced or characterized genome, whether partial or complete, of any organism or virus which may be used to reference identified sequences from a subject. A reference genome sometimes refers to a segment of a reference genome (e.g., a chromosome or part thereof, e.g., one or more portions of a reference genome). Human genomes, human genome assemblies and/or genomes from any other organisms can be used as a reference genome. One or more human genomes, human genome assemblies as well as genomes of other organisms can be found at the National Center for Biotechnology Information at www.ncbi.nlm.nih.gov. A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. In some embodiments, a reference genome comprises sequences assigned to chromosomes. The term “reference sequence” as used herein refers to one or more polynucleotide sequences of one or more reference samples. In some embodiments reference sequences comprise sequence reads obtained from a reference sample. In some embodiments reference sequences comprise sequence reads, an assembly of reads, and/or a consensus DNA sequence (e.g., a sequence contig). In some embodiments a reference sample is obtained from a reference subject substantially free of a genetic variation (e.g., a genetic variation in question). In some embodiments a reference sample is obtained from a reference subject comprising a known genetic variation. The term “reference” as used herein can refer to a reference genome, a reference sequence, reference sample and/or a reference subject. In some embodiments, sequence reads can be found and/or aligned with sequences in nucleic acid databases known in the art including, for example, GenBank, dbEST, dbSTS, EMBL (European Molecular Biology Laboratory) and DDBJ (DNA Databank of Japan). BLAST or similar tools can be used to search the identified sequences against a sequence database

In certain embodiments, mappability is assessed for a genomic region (e.g., portions, genomic portions). Mappability is the ability to unambiguously align a nucleotide sequence read to a portion of a reference genome, typically up to a specified number of mismatches, including, for example, 0, 1, 2 or more mismatches. In some embodiments, mappability is provided as a score or value where the score or value is generated by a suitable mapping algorithm or computer mapping software. High quality sequence reads aligned to genomic regions comprising stretches of unique nucleotide sequence sometimes have a high mappability value.

Paired-end reads are sometimes mapped to opposing ends of the same polynucleotide fragment, according to a reference genome. In some embodiments only one read of a read mate pair is mapped to a reference genome. In some embodiments, read mates of a read mate pair are mapped independently. In some embodiments, information from both read mates of a read mate pair (e.g., orientation, estimated insert size, estimated distance between reads) is factored in the mapping process. A reference genome is often used to determined and/or infer the sequence of nucleic acids located between read mate pairs. A nucleic acid located between two paired-end reads is often referred to herein as an insert. In some embodiments insert size is determined or estimated by mapping both read mates of a read mate pair to a reference sequence. In some embodiments insert size (e.g., length) is estimated or determined according to a distribution. In certain embodiments the probability of an insert size comprising a viable insert is determined from the insert size distribution. In some embodiments insert size is determined by a suitable distribution and/or a suitable distribution function. In some embodiments insert size or an estimated insert size is determined by an insert size distribution component which often comprises a distribution function. Non-limiting examples of a distribution function include a probability function, probability distribution function, probability density function (PDF), a kernel density function (kernel density estimation), a cumulative distribution function, probability mass function, discrete probability distribution, an absolutely continuous univariate distribution, the like, any suitable distribution, or combinations thereof. Insert size is sometimes generated from averaged, normalized and/or weighted insert lengths. Insert size distributions are sometimes estimated according to estimated and/or known nucleic acid fragment lengths derived from fragments of a nucleic acid library that was sequenced. In some embodiments a suitable storage medium comprises stored estimated insert lengths, insert length distributions and the like. In certain embodiments, sequence reads comprise an insert size distribution, estimated insert lengths, estimated distances between read mates, the like or combinations thereof.

Read Recruitment

In some embodiments a method, process or system herein comprises a read recruitment process. A read recruitment process is often carried out by a read recruitment component. In certain embodiments a read recruitment process comprises obtaining and/or selecting sequence reads as described herein. In some embodiments a read recruitment process comprises a method of obtaining and/or selecting a subset of reads from a plurality of reads.

In some embodiments one read mate of a read mate pair (e.g., obtained from a paired-end sequencing approach) maps to a reference genome and the other read mate of the read mate pair is mapped incorrectly to the reference genome, fails to map to the reference genome or comprises a low mappability score. Such a read mate pair is sometimes referred to as a discordant read mate pair. In some embodiments a discordant read mate pair comprises one read mate that maps to a region of a reference genome of interest (e.g., a genomic regions of interest) and the other read mate fails to map to a portion of the reference genome of interest. In some embodiments a discordant read mate pair comprises a first read mate that maps to a portion of a reference genome of interest (e.g., a portion of a genomic region of interest) and a second read mate that maps to an unexpected location of a reference genome. Non-limiting examples of an unexpected location of a reference genome include (i) a different chromosome than the chromosome to which the first read mapped, (ii) a genomic location separated from the first read mate by more than a predetermined distance, non-limiting examples of which include a distance predicted from an estimated insert size; a distance of more than 300 bp, more than 500 bp, more than 1000 bp, more than 5000 bp, or more than 10,000 bp and (iii) an orientation inconsistent with the first read (e.g., opposite orientations), the like or a combination thereof. In some embodiments a discordant read mate pair comprises a first read mate that maps to a first segment of a reference genome, or a portion thereof, and a second read mate that is unmappable and/or comprises low mappability (e.g., a low mappability score). In some embodiments a discordant read mate pair comprises a first read mate that maps to a first segment of a reference genome, or a portion thereof, and a second read mate, where the mappability of the second read mate, or a portion thereof, is not determined. Discordant read mate pairs can be identified by a suitable discordant read identifying component or by a machine comprising a discordant read identifying component, which discordant read identifying component generally identifies discordant read mate pair. Non-limiting examples of discordant read identifying components include SVDetect, Lumpy, BreakDancer, BreakDancerMax, CREST, DELLY, the like or combinations thereof. In some embodiments discordant read mate pair are not identified by an algorithm or component. In certain embodiments discordant read pairs are identified by an algorithm that identifies paired-end read mates, where one read mate of a read mate pair maps to a reference genome and the other read mate of the read mate pair is mapped incorrectly to the reference genome, fails to map to the reference genome or comprises a low mappability score.

In some embodiments a read recruitment process selects and/or obtains all paired end reads (e.g., from a plurality of reads) that map to a reference genome in a genomic region of interest. In some embodiments all paired end reads, where at least one of each read mate pair maps completely or partially to a reference genome, in a genomic region of interest, are obtained and/or used for an analysis herein. In some embodiments all paired end reads, wherein at least one or both of each read mate pair maps completely or partially to a reference genome in a genomic region of interest, are obtained and/or used for an analysis herein. In some embodiments all discordant read mate pairs, wherein at least one of the reads of each discordant read mate pair maps to a reference genome in a genomic region of interest, are obtained and/or used for an analysis herein.

In some embodiments a method or system herein comprises obtaining a set of paired-end sequence reads comprising a plurality of read mate pairs. In some embodiments a method or system herein comprises obtaining a set of paired-end sequence reads consisting of a plurality of read mate pairs. In certain embodiments each pair of sequencing reads of a read mate pair are obtained from a paired end sequencing approach. In certain embodiments each pair of sequencing reads of a read mate pair consists of two read mates. A read mate is often a sequencing read. In some embodiments a method or system herein comprises obtaining a set of paired-end sequence reads comprising a plurality of read mates pairs, where at least one of the read mates of each pair, or a portion thereof, is mapped to at least a portion of a reference genome comprising a pre-selected genomic region of interest and where some of the paired-end sequence reads are not mapped to the at least one portion of the reference genome comprising the pre-selected genomic region of interest.

In some embodiments methods and systems herein circumvent read mapping problems in regions comprising expanded STR's, sequence junctions and large complex variations, by recruiting both read mates of a read mate pair obtained from paired end sequence reads where a first read of a read mate pair maps to a genomic region of interest, regardless of the mappability of a second read of a read mate pair. In certain embodiments methods and systems herein utilize the location of mapped read mates, the orientation of both read mates of a read mate pair, and/or estimated distances between read mates (e.g., estimated insert sizes) to assemble regions of genomic nucleic acid obtained from a subject that may comprise genetic variations.

In certain embodiments methods and systems herein use one genomic region of interest to which reads are mapped. In certain embodiments methods and systems herein use two genomic regions of interest, which might have been identified using split-read signal or discordant mate signal, to recruit and/or retrieve reads located at or near genetic variations comprising translocations and/or junctions. In some embodiments a genomic region of interest is preselected (e.g., prior to obtaining reads, prior to recruiting reads, prior to analysis, mapping and/or assembly of reads). A genomic regions of interest can be any suitable portion of a genome. A genomic region of interest can comprise or consist of one or more chromosomes, genes, exons, introns, untranslated regions (e.g., regulatory regions, promoter/enhancer regions), methylated regions, unmethylated regions, or portions thereof. In some embodiments a genomic region of interest comprises a region suspected of having a genetic variation or a region that may contain a known genetic variation (e.g., a genetic variation previously identified in another subject or sub-population). In some embodiments a genomic region of interest comprises a genetic variation. In some embodiments a genomic region of interest does not comprises a genetic variation.

Sequence reads (e.g., read mates) often comprise a known orientation. For example, a storage medium often comprises a file which contains a known orientation of read mates. In some embodiments an orientation of read mates and/or an estimated insert size is used to determine the position of a mapped, unmapped, poorly mapped or discordant read mate within a pile-up, contig and/or supercontig.

In some embodiments, sequence reads are trimmed. In certain embodiments trimming refers to identification and/or removal of synthetic and/or heterologous nucleic acids, or portions of nucleic acids from sequence reads, which synthetic and/or heterologous nucleic acids were used in construction of a library and/or for a sequencing method. Heterologous nucleic acids are often heterologous or foreign to a subjects genome. Non-limiting examples of synthetic and/or heterologous nucleic acids that are often trimmed include adapters, plasmids, vectors, primer binding sites, index tags (e.g., nucleic acid barcodes sequences), nucleic acid capture sequences, the like or combinations thereof. In some embodiments trimming comprises instructing a processor to delete and/or ignore those portions of sequencing reads that are synthetic and/or heterologous. Synthetic nucleic acids, heterologous nucleic acids and/or trimmed nucleic acids are often not included in method or process herein. In some embodiments sequence reads are trimmed prior to, or during, obtaining a set of paired-end sequence reads. In some embodiments sequence reads are trimmed prior to, or during, determining a pile-up relationship, filtering, constructing one or more contigs, assembling one or more supercontigs and/or generating a genotype likelihood ratio. In certain embodiments trimming is performed by a trimming component.

Pile-Up Relationships

In some embodiments a method or process herein comprises determining a pile-up relationship for a set or subset of sequence reads. In some embodiments a pile-up relationship comprises one or more overlaps (e.g., a plurality of overlaps) between a plurality of reads of a set wherein some of the reads map to a region of a reference genome of interest. In some embodiments a pile-up relationship comprises constructing a tiling graph. In some embodiments a pile-up relationship comprises all reads of a set of paired-end sequence reads. In some embodiments a pile-up relationship comprises selected reads of a set of paired-end sequence reads. In some embodiments an overlap comprises an alignment of two or more reads. In certain embodiments an overlap comprises an alignment score. In certain embodiments an overlap is determined according to a k-mer hashing strategy.

In some embodiments a pile-up relationship comprises a plurality of overlaps. In certain embodiments a pile-up relationship comprises one or more overlaps that are selected and/or stored (e.g., stored into memory). Sometimes determining a pile-up relationship includes determining and/or evaluating all possible overlaps between a plurality of reads. In certain embodiments, only some overlaps, of all possible overlaps, are selected and/or stored. In certain embodiments, all overlaps that are selected are stored and are used for a pile-up relationship.

Overlaps that are used for a pile-up relationship often meet one or more criteria. For example, in some embodiments a first criteria includes an overlap between a first read and a second read that is above an alignment threshold score. In some embodiments, an overlap is selected and/or stored where a first read of a set comprises an overlap with a second read of a set and the overlap comprises an alignment score that is greater than a predetermined alignment score threshold or cutoff. In some embodiments, an overlap is selected and/or stored where a first read of a set comprises an overlap with one, two, three or more other reads of a set and the overlap comprises an alignment score that is greater than a predetermined alignment score threshold. In some embodiments, an overlap is selected and/or stored where a first read of a set comprises an overlap with one, two, three or more other reads of a set, each of the overlaps comprises an alignment score that is greater than a predetermined alignment score threshold and each of the overlaps comprises an identical alignment score. An alignment score can be determined by any suitable method or algorithm, non-limiting examples of which include the method of Smith and Waterman (Smith T F, Waterman M S., (1981) J. Theor. Biol. 91(2):379-80; and Smith T F, Waterman M S., (1981) J. Mol. Biol. 147(1):195-7) and Needleman (Needleman, S. B. and Wunsch, C. D. (1970) J. Mol. Biol. 48(3):443-53). For example, in some embodiments the algorithm of Smith-Waterman is used with an alignment score cutoff of 500 where the match score is 10 and mismatch penalty is −500. In certain embodiments, insertions and deletions (in/dels) are prohibited and/or excluded in a read-read alignments. In some embodiments the penalty for initiating or extending insertions or deletions is set sufficiently high to exclude all or most in/dels. In some embodiments, gaps are not allowed. In certain embodiments, some in/dels can be allowed or included in a read-read alignments.

In some embodiments a second criteria requires an overlap to be the highest alignment score of all possible overlaps. In some embodiments, an overlap that is selected and/or stored comprises a highest alignment score of all possible overlaps (e.g., all possible alignments) between a first read and any other read of a set of reads. Sometimes an overlap that is selected and/or stored comprises a highest alignment score of a plurality of overlaps (e.g., a plurality of alignments) determined between a first read and a plurality of other read.

In some embodiments an overlap extends one or more nucleotides past a 3′ end or a 5′ end of a read. In some embodiments a third criteria requires an overlap to extend a first read past a 5′ or 3′ end of the first read. In certain embodiments a first read comprises an overlap that extends the first read in either a 5′ or 3′ direction and past an end of the first read. An overlap between a first read and a second read that extends the first read often comprises one or more nucleotides of a second read that extend past a 3′ end or a 5′ end of the first read. Sometimes an overlap is selected or stored when a first read and a second read overlap, and the overlap extends the first read past the 3′ or 5′ end of the first read. In some embodiments an overlap extends at least 1, 2, 3, 4, 5, 6, 7, 8, 9, 10, 15, 20, 50, 100 or at least 150 nucleotides past a 3′ end or a 5′ end of a read. In certain embodiments a first read comprises a first overlap with a second read that extends the first read in a 3′ direction and the first read comprises a second overlap with a third read that extends the first read in a 5′ direction. In certain embodiments a pile-up relationship comprises on overlap between a first read and a second, and an overlap between the first read and a third read where the overlaps extend the first read in the 3′ and the 5′ direction.

In some embodiments a pile-up relationship comprises additional selected overlaps for a first read, a second read and, for example, a third read. For example, a first read often comprises a first overlap with a second read that is selected and/or stored when the second read includes an overlap with a third read that extends the second read. In the foregoing example, the overlaps would extend the first read and the second read in the same 3′ or 5′ direction. Furthermore, the third read may, or may not overlap with the first read. In some embodiments a first read comprises a plurality of overlaps with a plurality of reads that extend the first read in a 5′ and/or a 3′ direction where each overlap meets one or more of the criteria. In some embodiments a first read comprises at least two overlaps that extend past the 5′ end of the first read and at least two overlaps that extend past the 3′ end of the first read.

In some embodiments a pile-up relationship comprises a plurality of selected and/or stored overlaps for a plurality of reads where each overlap may be selected from a plurality of overlaps. In some embodiments a pile-up relationship comprises a plurality of selected and/or stored overlaps for a set of reads where each overlap satisfies the following: (i) the overlap must comprise an alignment between a first read and a second read where the alignment score is higher than a predetermined alignment score threshold, (ii) the overlap between the first read and the second read must extend the first read past the 3′ end or the 5′ end of the first read and (iii) the overlap between the first read and the second read include a highest alignment score of all possible overlaps that satisfy (i) and (ii) between the first read and any other read of a set of reads. In certain embodiments, in addition to (i), (ii) and (iii) above, the second read comprises an overlap that (iv) is higher than a predetermined alignment score threshold, (v) extends the first read and the second read in the same 3′ or 5′ direction and past the end of the second read and (vi) is the highest alignment score between the second read and any other read that satisfies (iv) and (v) above. In certain embodiments a method or process comprises determining a pile-up relationship comprising selecting and/or storing overlaps of a plurality of reads of a set where each overlap satisfies (i), (ii), and (iii) above. In some embodiments each read of a set comprises 1, 2, 3, 4, 5, 6, 7, 8, 9, 10, 11, 12, 13, 14, or 15 or more overlaps that extend the read to in the 5′ and/or 3′ directions. A pile-relationship often comprises a plurality of reads, each comprising a plurality of overlaps.

In some embodiments an overlap comprises a score or index. For examples, in certain embodiments all possible overlaps for a set of reads are determined and sometimes each overlap is associated with a score or value. A score or value (e.g., a point value) associated with an overlap is sometimes a sum, average or mean value determined from one or more of conditions (i), (ii), (iii), (iv), and/or (v) above. In some embodiments an overlap is associated with an alignment score. In certain embodiments, overlaps are filtered. Overlaps that are filtered are often removed or deleted from a pile-up relationship. Overlaps that are deleted or filtered are often not considered for de novo assembly of a contig or supercontig. In some embodiments overlaps are filtered according to a score or a predetermined cut-off score. In some embodiments overlaps are filtered according to a predetermined alignment score threshold. In some embodiments overlaps that do not meet the requirements of some or all of (i), (ii), (iii), (iv) and (v) are filtered. Filtering algorithms are known and any suitable filter can be modified to filter overlaps of a pile-relationship. In some embodiments a filter comprises a pruning algorithm that iterates over all reads in a set and maintains a list of overlaps for each read (e.g., according to (i), (ii), (iii), (iv) and/or (v)) that are being selected and/or stored. In certain embodiments a program instructs a microprocessor to filter a plurality overlaps for a set of reads.

In certain embodiments, determining a pile-up relationship does not comprise a process that includes error correction. In some embodiments a pile-up relationship does not comprise overlaps that comprise insertions or deletions. In some embodiments a pile-up relationship comprises overlaps that comprise one or more mismatches.

Contigs

In some embodiments one or more contigs are assembled and/or constructed for a set of reads. In some embodiments one or more contigs are constructed according to a plurality of overlaps that are selected and/or stored for set of reads. In certain embodiments one or more contigs are constructed according to a pile-up relationship comprising a plurality of overlaps for a set of reads. In certain embodiments contigs are constructed from one or more starter reads. In certain embodiments one or more contigs are constructed from 1, 2, 3, 4, 5, 6, 7, 8, 9 or 10 or more starter reads. A starter read can be any suitable read of a set. Sometimes a starter read comprises the most 5′ read and/or the most 3′ read of a set of reads. The most 5′ read is often a read mapped to the most 5′ region of a genomic region of interest to which some or all sequence reads of a set are mapped. Likewise, the most 3′ read is often a read mapped to the most 3′ region of a genomic region of interest to which some or all sequence reads of a set are mapped. In certain embodiments a contig is assembled from a starter read that is not the most 3′ or 5′ read of a set.

In some embodiments a contig is assembled from a starter read and the process comprises iteratively adding at least one nucleotide to a position 3′ or 5′ of a starter read. A position 3′ or 5′ of a starter read can be a position 3′ or 5′ of any suitable nucleotide of a starter read. In some embodiments a position 3′ or 5′ of a starter read is a position 3′ or 5′ of an end of a starter read (e.g., a 3′ end or a 5′ end). In some embodiments a position 3′ or 5′ of a starter read is a position 3′ or 5′ of a median or middle nucleotide of a starter read. Often, a process of iteratively adding at least one nucleotide to a position 3′ or 5′ of a starter read comprises first selecting a suitable position within a starter read (e.g., a nucleotide located at a suitable position), determining a majority consensus nucleotide for the selected position (e.g., see below for determining a majority consensus nucleotide) according to a pile-up relationship and iteratively adding one or more nucleotides to the 3′ and/or 5′ position of the majority consensus nucleotide that was determined according to the pile-up relationship, thereby initiated assembly of a contig. In certain embodiments, a starter read is a first read that will start a contig assembly process and a pileup relationship of recruited reads determines the majority consensus nucleotide for each nucleotide position of a starter read. For example, in certain embodiments a starter read is re-assembled by a similar process to that used for assembly of a contig or intermediate contig.

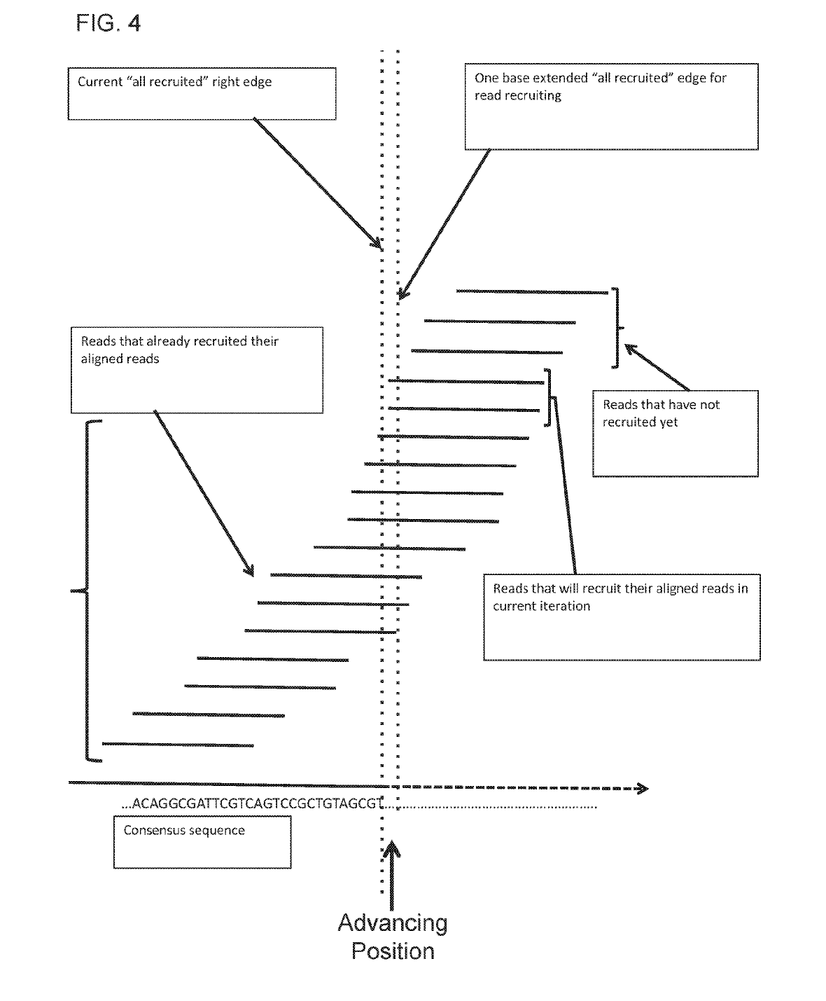

In some embodiments a contig is assembled from a starter read and the process comprises iteratively adding at least one nucleotide to a position 3′ or 5′ of an intermediate contig. In some embodiments an intermediate contig comprises a starter read (e.g., at least some nucleotides of a starter read) and one or more nucleotides added to the 3′ and/or 5′ side of the starter read. In some embodiments an intermediate contig comprises some or all of the nucleotides of a starter read. A position 3′ or 5′ of a starter read or intermediate contig often is the nucleotide position immediately adjacent to and past the 3′ or 5′ end of an in silico assembled nucleic acid sequence of a starter read or intermediate contig. In some embodiments a nucleotide position located immediately adjacent to and past the 3′ or 5′ end of a starter read or intermediate contig, where a majority consensus nucleotide has not yet been added (e.g., not yet added during an in silico contig assembly process), is referred to herein as an advancing position (e.g., see FIG. 4). In some embodiments a position 3′ or 5′ of a starter read, where the position 3′ or 5′ of the starter read (e.g., 3′ or 5′ of a nucleotide within a starter read) that has not yet been filled by a majority consensus nucleotide, is referred to as an advancing position. In certain embodiments an intermediate contig comprises a starter read and one or more nucleotides added to a position 3′ or 5′ of the starter read. A nucleotide is often added to a position 3′ or 5′ of a starter read or intermediate contig where that position (e.g., the advancing position) comprises a majority consensus nucleotide.

In some embodiments a majority consensus nucleotide is determined according to a plurality of overlaps or alignments determined according to a pile-up relationship. Sometimes one or more nucleic acid reads are aligned with a starter read, an intermediate contig or portions thereof, according to overlaps that were selected and/or stored. In certain embodiments selected and/or stored overlaps (e.g., overlapping reads) are recruited to an alignment comprising a starter read or intermediate contig where some or all of the reads or the overlaps include a nucleotide that overlaps or aligns with an advancing position. In certain embodiments, a majority consensus nucleotide is determined according to nucleotides that overlap or align with an advancing position. In some embodiments a majority consensus nucleotide is a nucleotide (e.g., A, T, C, G or U) located at or aligned to an advancing position where at least 3, at least 4, at least 5, at least 6, at least 7, at least 8, at least 9, at least 10, at least 15, at least 20, at least 30, at least 50, at least 100 or at least 200 of the overlapping reads include the same nucleotide (e.g., A, T, G, C or U) at the advancing position. In some embodiments a majority consensus nucleotide is a nucleotide (e.g., A, T, C, G or U) located at or aligned with an advancing position where at least 5%, at least 6%, at least 7%, at least 8%, at least 9%, at least 10%, at least 15%, at least 20%, at least 25%, at least 30%, or at least 50% of the overlapping reads include the same nucleotide (e.g., A, T, G, C or U) at the advancing position.

In some embodiments of a contig assembly, an advancing position includes a single majority consensus nucleotide, the majority consensus nucleotide is added to a position 3′ or 5′ of the starter read or intermediate contig and the in silico process is repeated for the next advancing position. In some embodiments, an advancing position includes a polymorphic base position, for example where more than one majority consensus nucleotide exists for the advancing position (e.g., the polymorphic base position). Where two majority consensus nucleotides are identified for a polymorphic base position, a copy is often made of the intermediate contig resulting in two identical intermediate contig copies. In this situation one of the two majority consensus nucleotides identified is added to the advancing position of one of the two copies and the other majority consensus nucleotide identified is added to the advancing position of the other copy. This process is sometimes referred to as splitting or splitting a contig. In some embodiments a system, method, process or algorithm herein comprises a method of splitting one or more contigs. In some embodiments a computer program component (i.e., component) provides instructions to a microprocessor to split one or more contigs.

In certain embodiments where three majority consensus nucleotides are identified for an advancing position (e.g., a polymorphic base position), two copies of the intermediate contig are generated resulting in 3 identical contigs and one of each of the three majority consensus nucleotides is added to the advancing position of each of the three identical contigs. In such a situation, a different nucleotide is added to each of the three identical contigs. In other words, the contig is split into three contigs. Likewise, where four majority consensus nucleotides are identified for an advancing position (e.g., a polymorphic base position), often three copies of the intermediate contig are generated resulting in 4 identical contigs, and one of each of the four majority consensus nucleotides is added to the advancing position of each of the four identical contigs. In other words, the contig is split into four contigs. In certain embodiments an intermediate contig comprises a split contig (e.g., a contig that resulted from splitting a contig).

In certain embodiments during the process of assembling a contig, a contig or an intermediate contig is split multiple times. For example, during the assembly of a contig or intermediate contig, a first polymorphic base position and a second polymorphic base position may be encountered where the first polymorphic positions results in a first splitting of the contig and the second polymorphic base position can result in a second splitting of a contig. For example, an intermediate contig can be split 1 or more, 5 or more or 50 or more times. In some embodiments an intermediate contig is split 1 to 500, 1 to 100, 1 to 50, 1 to 25 or 1 to 10 times. In some embodiments an intermediate contig is not split. In certain embodiments a second polymorphic base position is encountered during the assembly of an intermediate contig that resulted from a first split (e.g., an intermediate contig resulting from a previous split where a first polymorphic base position was encountered). In this situation, an intermediate contig can be split again or the contig may not be split. If the contig was previously split at some position (e.g., a first polymorphic position), the splitting process determines if any read pair or set of read pairs overlap with both the first polymorphic position and the second, currently encountered, polymorphic base position (e.g., the advancing position where two or more majority consensus nucleotides align). In some embodiments, if such a set of overlapping read pairs exists, and the set of read pairs include (i) the first polymorphic base that was added in the first polymorphic position and (ii) a single majority consensus nucleotide (e.g., the same nucleotide) at the second polymorphic base position, then the majority consensus nucleotide for the second polymorphic base position is added to the intermediate contig strand at the advancing position and the contig is not split. Further, the set of reads above that satisfied the conditions of both (i) and (ii) are not used to split any other contig and are not used to assemble another contig. In some embodiments, if such a set of overlapping read pairs exists, and the set of read pairs include (i) the first polymorphic base that was added in the first polymorphic position and (iii) two or more majority consensus nucleotides at the second polymorphic base position, then the intermediate contig is split again. In certain embodiments, a set of overlapping read pairs that do not satisfy condition (i), but provide a majority consensus nucleotide for the second polymorphic position are not used to split the intermediate contig in the examples above and such read pairs are excluded from assembling the intermediate contig in the examples above. The rationale behind this design is to prevent splitting at a polymorphic base where a haplotype that includes that polymorphic base has already been included in the assembly of another contig. In the example above, if condition (i) is satisfied and the set of reads that overlap with the first polymorphic base position include two or more majority consensus nucleotides for the second polymorphic base position, then the contig will be split accordingly, in some embodiments. Likewise, in some embodiments, if condition (i) is not satisfied, then the contig will be split. Additional details for splitting are described in example 1.

In some embodiments a graph cycle is detected by duplicating a split that was already taken in the contig. In certain embodiments, if repeated splits are detected, the contig is labeled as “bad” and assembly of the “bad” contig is terminated. In certain embodiments, contigs labeled as “bad” are not used in supercontig construction.

In some embodiments a read of a set of reads is used only one time for the construction of a contig. In certain embodiments, a read that comprises a majority consensus nucleotide that is incorporated into an advancing position of a contig, is not used to add additional nucleotides to another contig. In some embodiments, where a contig is copied, due to the presence of two or more majority consensus nucleotides, a read will only be used to continue building one of the contig copies. In some embodiments, a read can be re-used in distinct contigs.

In some embodiments if a polymorphic position is encountered that was already encountered and split on in some other contig, then the contig splitting in this contig is not performed, but rather just referenced in this contig as a “duplicate” split. In such an embodiment, the duplicate split contains the same set of consensus bases and the same set of reads supporting them at the same positions in the reads. In such an embodiment, once all contigs are assembled then these skipped splits are reintroduced by adding all possible endings of the consensus sequence from the contig with the duplicate splits and other contigs split-off from that contig after the “duplicate split” position. In certain embodiment it is assumed that once the same set of reads piled-up the same way is encountered during a contig building process that the consensus sequences after that position will be identical because these reads will recruit the same set of reads afterwards. In some embodiments this “duplicate” split detection should not change the assembled contigs, but just speed-up the contig assembly computational process.

In some embodiments if a split-off contig cannot recruit any new reads to extend the contig, while some of the other contigs resulting from this spit position can recruit new reads, such contig is labeled as “dead-end”. These contigs are often a result of following a consensus base resulting from a systematic sequencing error rather than from a true polymorphism. In some embodiments these “dead-end” contigs are discarded.

In some embodiments contigs that at are assembled from less than a predetermined amount of reads, or from a pile-up relationship containing less than a predetermined amount of reads, are discarded or removed. In some embodiments a predetermined amount of reads is about 200 reads or less, 100 reads or less, 50 reads or less, 25 reads or less or 10 reads or less. In certain embodiments, contigs that are assembled from less than a predetermined amount of reads are discarded, deleted and/or removed by a filter. In some embodiments contigs that are discarded, deleted and/or removed are not used for assembly of a supercontig.

Supercontigs

Contigs assembled in previous step can either span an entire genomic region of interest or may terminate, for example in places where coverage drops off or where high read error rates (e.g., usually systematic errors) prohibit high-scoring overlaps. In certain embodiments a contig that spans an entire genomic regions of interest is a supercontig and requires no additional assembly. A supercontig often spans an entire genomic region of interest. Contigs that do not span an entire genomic region of interest can be assembled into supercontigs. In some embodiments, one or more supercontigs are assembled from two or more contigs. In certain embodiments, read mates (e.g., of a read mate pair) are used to link contigs together to form supercontigs. For example, in some embodiments a coverage gap between two neighboring contigs can be bridged by read mates of a read mate pair where a first read mate of the pair provides an overlap with a first contig and the second read mate of the pair provides an overlap with the other contig. Read mates of a pair that bridge or join two neighboring contigs can provide information as to the estimated distance between the contigs, the order and orientation of the contigs. For example, the estimated insert length between the read mates can provide an estimated distance between two bridged contigs. Sometimes the orientation of read mates that bridge two contigs provide the relative orientation and order of the two bridged contigs to each other. In some embodiments a first contig is joined to a second contig according to a plurality of read mate pairs. In some embodiments a first contig is joined to a second contig according to at least 3, at least 4, at least 5, at least 6, at least 7, at least 8, at least 9, at least 10, at least 15, at least 20, at least 30, or at least 50 read mate pairs.

In certain embodiments, once two contigs are linked by one or more read mates, additional reads, overlaps (e.g., as determined according to a pile-up relationship) and/or contigs can be recruited and/or aligned to assemble the intervening sequence between neighboring contigs that were bridged.

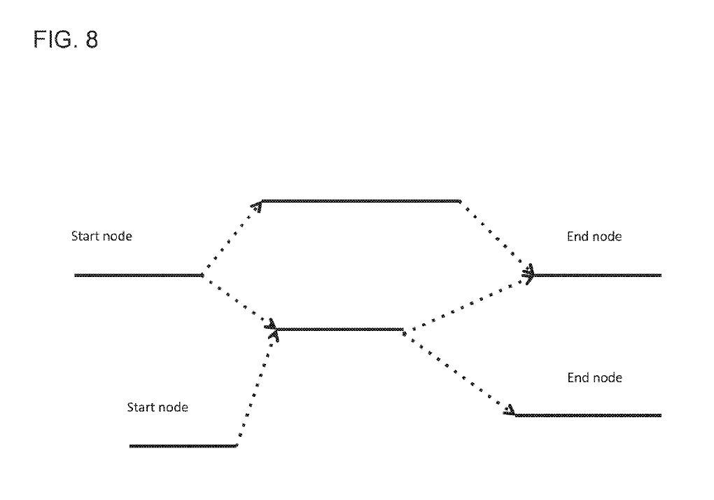

In certain embodiments supercontig construction involves constructing a graph with contigs as vertices and the identified links (e.g., read mates that link two contigs) as oriented edges. In some embodiments the oriented edges are recorded where two neighboring contigs are bridged by a minimum number of read mate pairs where the minimum number of read mate pairs is at least 3, at least 4, at least 5, at least 6, at least 7, at least 8, at least 9, at least 10, at least 15, at least 20, at least 30, or at least 50 read mate pairs. In some embodiments the minimum number of read mate pairs required to bridge two neighboring contigs is at least 5%, at least 6%, at least 7%, at least 8%, at least 9%, at least 10%, at least 15%, at least 20%, at least 25%, at least 30%, or at least 50% of the mean contig coverage. Mean contig coverage refers to the mean number of reads spanning each nucleotide position of a contig or intermediate contig. For example, the number of reads overlapping each nucleotide position in a contig is often computed as a position coverage and the average of the position coverages over all positions in a contig is the mean contig coverage. In some embodiments read mate pairs that bridge two contigs share the same orientation. In certain embodiments two more contigs are bridged, thereby forming supercontigs, by traversing all paths through the graph, while avoiding cycles, starting from all vertices with in-degree 0 and ending with vertices with out-degree 0 (e.g., see FIG. 8). In certain embodiments, contigs that are not connected to any other contigs (e.g., have both in-degree 0 and out-degree 0) create a supercontigs with just one contig.

Haplotyping

In some embodiments the supercontigs that are assembled by the above described processes represent all possible sequence arrangements, and thus represent all possible haplotype sequences (i.e., haplotypes). In some embodiments, haplotypes are combined directly by a caller, according to a predetermined ploidy, thereby producing all possible genotypes (e.g., genotype hypotheses, genotype likelihoods or genotype likelihood ratios). In some embodiments all haplotypes are subjected to a haplotyping process prior to being processed by a caller. In some embodiments a haplotyping process initiates objects associated with each haplotype (e.g., haplotype objects). Haplotype objects can include mapping weights, identified false junctions and/or identified false insertions. For example, in certain embodiments a haplotyping process comprises a re-mapping of some or all reads to the haplotype sequences (e.g., supercontigs). In certain embodiments, this re-mapping includes a pre-computation of mapping weights described in the “Caller” section of Example I, wherein the mapping weights are associated with each haplotype. In certain embodiments a haplotyper process also performs identification of false junctions and false insertions in the haplotype sequences (see below). A haplotyper process is often conducted separate from the function of a caller to allow a caller component the opportunity to filter haplotypes, based on the output of the haplotyping process (e.g., haplotype objects, e.g., mapping weights, the identification of false junctions and/or false insertions), before they are combined to genotype sequence hypotheses. In some embodiments, identified false junctions and false insertions are listed in each haplotype object with necessary information determining their support by the reads. The caller component can then use cutoffs based on the attributes of the haplotype objects, such as false insertions, to filter and/or remove haplotypes. Any suitable cutoffs can be used.

False Junction Identification

In certain embodiments a haplotyper process comprises a method of identifying false junctions. In some embodiments false junctions are created due to false positive alignments. In some embodiments false junctions are comprised of sequence reads derived from different parts of the genome (e.g., parts of the genome outside of a genomic region of interest) that were recruited (e.g., obtained) and included in a contig assembly due to some sequence similarity.

Such a sequence similarity sometimes allows some reads to join certain sequences, however the junction would be sparsely covered. A haplotyper process can identify locations in a haplotype sequence where the number of read-pairs straddling a junction location is much lower than expected. In some embodiments a haplotyper process finds possible false junctions by computing the expected number of read mates some distance away (e.g., estimated from insert size distribution) and compares them to an observed count. Locations of low observed/expected ratios can be marked as possible false junctions. In some embodiments a suitable estimate of statistical fit (e.g., a chi-squared test) can be used to determine the significance of an observed-expected difference. In some embodiments false junctions are identified by using a centered or un-centered band around the mean of the insert size distribution to test for the false junctions. An interval of −20% and +80% of a band (e.g., currently size 50 for Illumina read-pair libraries) around the insert size distribution mean is sometimes used to compute the expected count and used to search for the observed count. In some embodiments a haplotyper process separately computes the observed and expected counts for forward reads and reverse reads (in the reverse direction) and then finds the local minima in the ratios. The haplotyper process sometimes reports all local minima that exceed specified ratio cutoff. In certain embodiments performing the search in both forward and reverse directions can give algorithmic confirmation of the junctions.

False Insertion Identification

In some embodiments a haplotyper processes comprises a false insertion detection process. In some embodiments a false insertion is an undesired or false insertion of foreign or misplaced nucleic acid sequences within an in silico assembled supercontig. In some embodiments a false insertion detection process determines the presence or absence of false insertions in a haplotype. In some embodiments a false insertion detection process determines a likelihood or probability that a false insertion is present or absent in a haplotype. In some embodiments a false insertion detection process marks, weights or scores potential false insertions and associates those objects with a haplotype. In some embodiments false insertions can be identified using pairing of false junctions (e.g., as described above). However, for identifying false insertions, a dedicated false insertion detection process is often more sensitive and specific than a false junction algorithm.

In some embodiments a false insertion detection process 1) identifies read mate-pairs that map to a haplotype where the distance between the read mates is larger than an estimated insert length (e.g., as determined by an insert size distribution component), thereby defining a hypothetical false insert between the read mates, and 2) determines if the hypothetical insert is occupied only by read mate pairs that are fully contained within the hypothetical insert. Read mates that occupy a hypothetical insert region are reads that contributed to the in silico assembly of the specified region. Any suitable method can be used to determine if read mates are fully contained within a hypothetical insert. For example, the medians or ends of the read mates that flank a hypothetical insertion can be used to define the beginning and the end of a hypothetical insert. In certain embodiments, the medians or ends of a collection of reads that overlap (e.g., according to a pile-up relationship) with the read mates that flank a hypothetical insertion can be used to define a hypothetical insertion region. Sometimes a combination of methods are used. For example, a false insertion process may first identify read mate-pairs that map to a haplotype where the distance between the median of read mates is larger than an estimated insert length (e.g., as determined by an insert size distribution component), thereby defining the hypothetical insert beginning and end according to the locations of the medians of the flanking read mates. If the algorithm determines that the hypothetical insert is occupied only by read mate pairs that are fully contained within the hypothetical insert, then, in some embodiments, the algorithm may re-define the hypothetical insert edges according to the edges or ends of the read mates that comprise the insert.

In some embodiments, a false insertion process re-computes for each base position within a hypothetical insert a measure of insert purity. If within the insert there are positions that are comprised mostly of insert reads (read-pairs that are fully contained within the proposed false insertion region) and are not contaminated by reads that cross the insertion boundary, or by reads which have mates crossing or outside the insertion boundary, then such insertion is recognized as false insertion. Any suitable process can be used to compute the insert purity for the base positions within a hypothetical insert and/or to define, redefine and/or confirm the length and/or edges of a false insertion region. For example, in some embodiments, each base position is re-computed by a contig assembly process according to overlaps and according to a pile-up relationship where the read mate pairs that are fully contained within the hypothetical insert are excluded from the assembly process. When such a method is used, base position that cannot be occupied by a majority consensus nucleotide are used to define and report the false insertion region. Any similar process can be used to define, redefine and/or confirm a false insertion region.

In some embodiments a haplotyper process marks, weights, penalizes or scores haplotypes that were determined to contain a false insertion. In some embodiments a haplotyper process marks, weights, or scores haplotypes that were determined not contain a false insertion. In some embodiments a caller uses the objects that a haplotyper process assigns to a haplotype to determine if a haplotype will be included in a genotype hypothesis.

Caller and Haplotype Likelihood Ratios

In some embodiments a caller process assembles genotypes and determines genotype likelihood ratios. A caller component often carries out a caller process. A caller (e.g., a caller component) can receive haplotypes from a supercontig assembly component and/or from a haplotyper (e.g., a haplotype component). In certain embodiments, a caller process combines haplotypes to generate all possible genotypes for a given ploidy. In some embodiments all possible genotypes for a given ploidy are assembled by a caller component (e.g., a “caller”). In some embodiments each possible genotype determined for a given ploidy is referred to as a genotype hypothesis. Haplotypes can be combined in all possible arrangements for a haploid, diploid, triploid subject or for a subject of any ploidy. For example, for a diploid sequence hypotheses all possible pairings of any two haplotypes, including homozygous arrangements consisting of two copies of the same haplotype, can be assembled by a caller, each of which is referred to as a genotype hypothesis.

In such diploid genotypes the haplotype contributions are 0.5 for each haplotype. In some embodiments haplotypes can be combined in any ratios, resulting in fractional haplotype contributions to genotypes. Such fractional genotypes can be used to genotype mosaic individual samples, or tumor samples that can reflect normal tissue contamination and/or tumor heterogeneity. In some embodiments every genotype assembled by a caller is individually a genotype hypothesis. Thus, in some embodiments a method and/or processes herein generates a genotype likelihood ratio according to the one or more haplotypes. In some embodiments a method and/or processes herein generates a genotype likelihood ratio according to the one or more haplotypes and their fractional contribution to the genotype. In some embodiments a method and/or processes herein generates a genotype likelihood ratio according to one or more genotype hypotheses. Thus, in some embodiments a caller process generates a genotype likelihood ratio according to the one or more haplotypes. In some embodiments a caller process generates a genotype likelihood ratio according to one or more genotype hypotheses (e.g., one selected genotype hypothesis). In some embodiments a caller process generates a genotype likelihood ratio according to a genotype hypothesis comprising a homozygous reference genome arrangement.

In certain embodiments, haplotypes obtained by a caller, from a haplotyper, are filtered (e.g., excluded) by a caller process, for example, according to the presence or absence of false junctions, false insertions and/or by mapping weights. Filtered haplotypes are often not used by a caller to assemble genotypes or to determine genotype likelihood ratios. In certain embodiments haplotypes are not filtered by a caller process.

In some embodiments the number of genotypes assembled for a genomic regions of interest represents all possible haplotype sequence arrangements for that region for a given ploidy. Any suitable number of genotypes can be assembled for a genomic region of interest. Sometimes a plurality of genotypes are assembled. Sometimes 1 or more genotypes are assembled. In certain embodiments, 1 to 100,000,000, 1 to 1,000,000, 1 to 100,000, 1 to 10,000, 1 to 1000, 1 to 500, 1 to 200, 1 to 50 or 1 to 20 genotypes are assembled for a genomic region of interest. In some embodiments at least 5, at least 10, at least 20, at least 30, at least 50, at least 100, at least 500 or at least 1000 genotypes are assembled for a genomic regions of interest.

In some embodiments a caller process determines a genotype of a genomic region of interest (e.g., for a subject) according to one or more genotype likelihood ratios. In some embodiments a caller process determines the most probable and/or most likely genotype of a plurality of possible genotype hypotheses according to one or more genotype likelihood ratios. In some embodiments a caller process can provide a list of genotype hypotheses to a health care professional or to an outcome component, where the list includes a probability, likelihood, a measure of statistical confidence, a measure of error, a ranking, the like, or a combination thereof that is associated with each genotype hypothesis. In some embodiments a caller process determines a genotype likelihood ratio according to one or more genotype hypothesis. In some embodiments a caller process determines one or more genotype likelihood ratios according to one or more genotype hypothesis.

In some embodiments a genotype likelihood ratio is determined according to equation 1

$$\frac{P ( G \vert \{ R \} )}{P ( {G}_{0} \vert \{ R \} )} = {\prod }_{\{ R \}} \frac{{\sum }_{\{ {A}_{G} \}} \frac{1}{{N}_{{A}_{G}}} {F}_{{A}_{G}} ( W ( R , {A}_{G} ) + α )}{{\sum }_{\{ {{A}_{G}}_{0} \}} \frac{1}{{N}_{{A}_{{G}_{0}}}} {F}_{{A}_{{G}_{0}}} ( W ( R , {A}_{{G}_{0}} ) + α )} \qquad Eq . 1$$

where G is a genotype sequence for a predetermined ploidy, G₀ is a reference sequence, {R} is a set of the read mate pairs R, N_{AG} is a number of alleles _{AG} in the genotype sequence _{G}, N_{AG0} is a number of alleles _{AG0} in the reference sequence G₀, and F_{AG} is a fraction of the alleles A_{G} in the genotype sequence G, F_{AG0} is a fraction of the alleles _{AG0} in the reference sequence G₀, W is a read-pair mapping weight, and a is a mapping probability constant. In some embodiments a genotype likelihood ratio is determined according to derivation of equation 1 or a variation of equation 1. The terms of Eq. 1 and derivations thereof are further described in Example 1.

In some embodiments the ploidy of a subject is known, predetermined or assumed. In some embodiments a method or process herein does not determine the ploidy of a subject. In some embodiments a method or process herein can determine an estimated ploidy of a subject, where the estimated ploidy is associated with a probability. In some embodiments a method or process herein can determine an estimated ploidy of a subject, where the estimated ploidy is associated with a maximum likelihood. In some embodiments the ploidy is diploid. In some embodiments a genotype probability is determined for a human subject that is diploid. For example, for a diploid genome the fraction of the alleles F_{AG} and F_{AG0} each equal a value of 0.5.

In some embodiments, the alpha value of Eq. 1 depends on a read-pair (e.g., mapping or mappability of a read pair). For example, if a read-pair has a second mapping outside of a contig assembly region or outside of a genomic region of interest then the alpha value is larger (e.g., comparable in value to the W). In some embodiments where the mappability of a read-pair is poor, alpha can correspond to a W value. In some embodiments, the default value of alpha for reads that do not have a second mapping (e.g., good mappability) can be from about 1e-5 or smaller, about 1e-10 or smaller, 1e-20 or smaller, about 1e-25 or smaller, about 1e-30 or smaller, about 1e-40 or smaller, about 1e-50 or smaller, about 1e-60 or smaller or about 1e-70 or smaller. In some embodiments, the default value of alpha for reads that do not have a second mapping (e.g., good mappability) is about 1e-50 or less. Additional details concerning alpha and W are provided in Example 1.

In some embodiments generating a genotype likelihood ratio comprises realigning and/or mapping of some or all reads that were obtained or recruited. In some embodiments reads are realigning and/or mapped to a reference (e.g., a reference haplotype or reference genotype hypothesis) by a caller component (e.g., a “caller”). In some embodiments reads are realigning and/or mapped to a reference (e.g., a reference haplotype or reference genotype hypothesis) by a haplotype component. In some embodiments generating a genotype likelihood ratio comprises realigning and/or mapping all reads to a reference genome. In some embodiments generating a genotype likelihood ratio comprises realigning and/or mapping all reads to one or more haplotypes. In some embodiments generating a genotype likelihood ratio comprises realigning and/or mapping all reads to one or more haplotypes. In some embodiments generating a genotype likelihood ratio comprises realigning and/or mapping all reads to one or more haplotypes (for example a genotype hypothesis) designated as a reference. Any suitable haplotype or genotype hypothesis can be a reference.

In some embodiments a plurality of genotype likelihood ratios are determined according to Equation 1. In some embodiments a genotype likelihood ratio is determined for a plurality of genotype hypotheses (e.g., possible genotypes). In some embodiments a genotype likelihood ratio is determined for a plurality of genotype hypotheses (e.g., possible genotypes) according to one or more haplotypes, or pairs of haplotypes that span the full length of a genomic region of interest. In certain embodiments, each genotype hypothesis is associated with a probability (e.g., a genotype likelihood ratio normalized by their sum).

In some embodiments the presence or absence of genetic alteration in a subject is determined according to a genotype likelihood ratio. In certain embodiments, a genotype hypothesis comprising the highest probability (e.g., highest genotype likelihood ratio), of all possible genotypes for a genomic region of interest, is the most probable genotype for a given genomic region of interest. In some embodiments the most probable genotype represents the nucleic acid sequence for one or more haplotypes of a genomic region of interest. In some embodiments the presence or absence of a genetic variation is determined according to a most probable genotype.

In some embodiments a genotype hypothesis with the highest likelihood ratio is used to make a call or determine an outcome. In some embodiments a genotype hypothesis with the highest likelihood ratio is used to determine the presence or absence of genetic alteration in a subject.

In some embodiments the highest likelihood ratio is determined according to a predetermined cut-off. In certain embodiments two or more likelihood ratios are determined to be the highest likelihood ratio and other parameters or data are used to determine an outcome or a genotype. In some embodiments the highest likelihood ratio values comprise a log-likelihood ratio of about 800 to 10,000. In some embodiments a highest likelihood ratio comprises a log-likelihood ratio of about 1000.

In some embodiments the likelihood ratio between the two top genotype hypotheses can be used to estimate the confidence in the presence or absence of genetic variations. In some embodiments the full set of genotype hypotheses can be evaluated for presence and absence of a genetic variation and the sets of hypotheses with the variation and without the variation can be used to determine the confidence in the presence of the variation in the sample.

Systems, Machines, Storage Mediums and Interfaces

Certain processes and methods described herein often cannot be performed without a computer, microprocessor, software, computer program component or other machine. Methods described herein typically are computer-implemented methods, and one or more portions of a method sometimes are performed by one or more hardware processors (e.g., microprocessors), computers, or microprocessor controlled machines. Embodiments pertaining to methods described in this document generally are applicable to the same or related processes implemented by instructions in systems, machines and computer program products described herein. Embodiments pertaining to methods described in this document generally can be applicable to the same or related processes implemented by a non-transitory computer-readable storage medium with an executable program stored thereon, where the program instructs a microprocessor to perform the method, or a part thereof. The descriptive term “non-transitory” as used herein is expressly limiting and excludes transitory, propagating signals (e.g., transmission signals, electronic transmissions, waves (e.g., carrier waves)). The terms “non-transitory computer-readable media” and/or “non-transitory computer-readable medium” as used herein comprise all computer-readable mediums except for transitory, propagating signals. In some embodiments, processes and methods described herein are performed by automated methods. In some embodiments one or more steps and a method described herein is carried out by a microprocessor and/or computer, and/or carried out in conjunction with memory. In some embodiments, an automated method is embodied in software, computer program components, microprocessors, peripherals and/or a machine comprising the like, that (i) obtain a set of paired-end sequence reads comprising a plurality of read mate pairs, each pair comprising two read mates, wherein at least one of the two read mates of each pair is mapped to at least one portion of a reference genome comprising a pre-selected genomic region of interest and wherein some of the paired-end sequence reads are not mapped to the at least one portion of the reference genome, (ii) determine a pile-up relationship for a set of sequence reads, (iii) construct one or more contigs according to a pile-up relationship, (iv) assemble one or more supercontigs, (v) generate a genotype likelihood ratio, (vi) determine the presence or absence of genetic alteration, or (vii) perform a combination thereof.

Machines, software and interfaces may be used to conduct methods described herein. Using machines, software and interfaces, a user may enter, request, query or determine options for using particular information, programs or processes (e.g., obtaining reads, recruiting reads, mapping reads, generating a pile-up relationship, constructing contigs, assembling haplotypes, generating a genotype likelihood ratio, determining the presence or absence of genetic alteration, the like or a combination thereof), which can involve implementing statistical analysis algorithms, statistical significance algorithms, statistical error algorithms, statistical probability algorithms, iterative steps, validation algorithms, and graphical representations, for example. In some embodiments, a data file may be entered by a user as input information, a user may download one or more data files by a suitable hardware media (e.g., flash drive), and/or a user may send a data set from one system to another for subsequent processing and/or providing an outcome (e.g., send sequence read data from a sequencer to a computer system for sequence read mapping; send mapped sequence data to a computer system for processing and yielding one or more genotype likelihood ratios).

A system typically comprises one or more machines. Each machine comprises one or more of memory, one or more microprocessors, and instructions. Where a system includes two or more machines, some or all of the machines may be located at the same location, some or all of the machines may be located at different locations, all of the machines may be located at one location and/or all of the machines may be located at different locations. Where a system includes two or more machines, some or all of the machines may be located at the same location as a user, some or all of the machines may be located at a location different than a user, all of the machines may be located at the same location as the user, and/or all of the machine may be located at one or more locations different than the user.

A system sometimes comprises a computing apparatus or a sequencing apparatus, or a computing apparatus and a sequencing apparatus (i.e., sequencing machine and/or computing machine). Apparatus, as referred to herein, is sometimes a machine. A sequencing apparatus generally is configured to receive physical nucleic acid and generate signals corresponding to nucleotide bases of the nucleic acid. A sequencing apparatus is often “loaded” with a sample comprising nucleic acid and the nucleic acid of the sample loaded in the sequencing apparatus generally is subjected to a nucleic acid sequencing process. The term “loading a sequence apparatus” as used herein refers to contacting a portion of a sequencing apparatus (e.g., a flow cell) with a nucleic acid sample, which portion of the sequencing apparatus is configured to receive a sample for conducting a nucleic acid sequencing process. In some embodiments a sequencing apparatus is loaded with a variant of a sample nucleic acid. A variant sometimes is produced by a process that modifies the sample nucleic acid to a form suitable for sequencing the nucleic acid (e.g., by ligation; e.g., adding adaptors to ends of sample nucleic acid by ligation, amplification, restriction digest, the like or combinations thereof). A sequencing apparatus is often configured, in part, to perform a suitable DNA sequencing method that generates signals (e.g., electronic signals, detector signals, data files, images, the like, or combinations thereof) corresponding to nucleotide bases of the loaded nucleic acid.

One or more signals corresponding to each base of a DNA sequence are often processed and/or transformed into base calls (e.g., a specific nucleotide base, e.g., guanine, cytosine, thymine, uracil, adenine, and the like) by a suitable process. A collection of base calls derived from a loaded nucleic acid often are processed and/or assembled into one or more sequence reads. In embodiments in which multiple sample nucleic acids are sequenced at one time (i.e., multiplexing), a suitable de-multiplexing process can be utilized to associated particular reads with the sample nucleic acid from which they originated. Sequence reads can be aligned by a suitable process to a reference genome and reads aligned to portions of the reference genome, and read mates that may not be aligned with a reference genome (e.g., read mates with low mappability scores or reads mates that are unmappable) can be stored and processed as described herein.

A sequencing apparatus sometimes is associated with and/or comprises one or more computing apparatus in a system. The one or more computing apparatus sometimes are configured to perform one or more of the following processes: obtain reads, recruit reads, filter reads, determine a pile-up relationship for a set of sequence reads, construct one or more contigs (e.g., contigs and/or intermediate contigs), assemble one or more supercontigs, filter contigs, filter haplotypes, perform one or more functions of a haplotyper, perform one or more functions of a caller, assemble one or more genotypes, generate one or more genotype hypotheses, generate one or more genotype likelihood ratios, determine the presence or absence of a genetic alteration, the like, or a combination thereof. The one or more computing apparatus sometimes are configured to perform one or more of the following additional processes: generate base calls from sequencing apparatus signals, generating reads, trim reads, de-multiplexing reads, align or map reads to a reference genome, and the like.

In some embodiments, a method or process is performed by multiple computing apparatus and a subset of the total processes performed by the system may be allocated to or divided among particular computing apparatus in the system. Subsets of the total number of processes can be divided among two or more computing apparatus, or groups thereof, in any suitable combination. A multi-computing apparatus system sometimes includes one or more suitable servers local to a sequencing apparatus, and sometimes includes one or more suitable servers not local to the sequencing apparatus (e.g., web servers, on-line servers, application servers, remote file servers, cloud servers (e.g., cloud environment, cloud computing)).

Apparatus in different system configurations can generate different types of output data. For example, a sequencing apparatus can output base signals and the base signal output data can be transferred to a computing apparatus that converts the base signal data to base calls. In some embodiments, the base calls are output data from one computing apparatus and are transferred to another computing apparatus for generating sequence reads. In certain embodiments, base calls are not output data from a particular apparatus, and instead, are utilized in the same apparatus that received sequencing apparatus base signals to generate sequence reads. In some embodiments, one apparatus receives sequencing apparatus base signals, generates base calls, sequence reads and de-multiplexes sequence reads, and outputs de-multiplexed sequence reads for a sample that can be transferred to another apparatus or group thereof that aligns the sequence reads to a reference genome. Output data from one apparatus can be transferred to a second apparatus in any suitable manner. For example, output data from one apparatus sometimes is placed on a physical storage device and the storage device is transported and connected to a second apparatus to which the output data is transferred. Output data sometimes is stored by one apparatus in a database, and a second apparatus accesses the output data from the same database.

In some embodiments a user interacts with an apparatus (e.g., a computing apparatus, a sequencing apparatus). A user may, for example, place a query to software which then may acquire a data set via internet access, and in certain embodiments, a programmable microprocessor may be prompted to acquire a suitable data set based on given parameters. A programmable microprocessor also may prompt a user to select one or more data set options selected by the microprocessor based on given parameters. A programmable microprocessor may prompt a user to select one or more data set options selected by the microprocessor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, machines, apparatuses (multiple apparatuses, also referred to herein in plural as apparatus), computer programs or a non-transitory computer-readable storage medium with an executable program stored thereon.

Systems addressed herein may comprise devices, peripherals, interfaces, storage media, sensors and parts of typical computer systems, such as, for example, network servers, laptop systems, desktop systems, handheld systems, personal digital assistants, cell phones, computing kiosks, and the like. A computer system may comprise one or more input means such as a keyboard, touch screen, mouse, voice recognition or other means to allow the user to enter data into the system. A system may further comprise one or more outputs, including, but not limited to, a display (e.g., CRT, LED or LCD), speaker, FAX machine, printer (e.g., laser, ink jet, impact, black and white or color printer), or any other sutiable output useful for providing visual, auditory and/or hardcopy output of information (e.g., outcome and/or report).

A computer system often comprises a user input component. A user input component facilitates entry and/or selection of information by subject, and/or other users. A user input component often facilitates entry and/or selection of information via user interface and/or other interface devices. For example, a user input component may cause a user interface to display one or more views graphical views to a user which facilitates entry and/or selection of information by the user. In some embodiments, a user input component is configured to facilitate entry and/or selection of information via one or more user interfaces associated with one or more users. In some embodiments, a user input component is be configured to facilitate entry and/or selection of information through a website, a mobile app, a bot through which text messages and/or emails are sent, and/or via other methods. In some embodiments, entered and/or selected information includes information related to nucleic acid sequences, users, samples and optional parameters that provide additional instructions to microprocessor. In some embodiments, a user input component is configured to prompt a subject or user and/or other users to answer specific questions, and/or provide other information. In some embodiments, a user input component is configured to associate a time of day, a duration of time, and/or other time related information with other entered, selected, stored, extracted and/or processed information.

In a system, input and output means may be connected to a central processing unit which may comprise a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by a provider, or it may be implemented as an internet based service where the user accesses a web page to enter and retrieve information. Accordingly, in certain embodiments, a system includes one or more machines, which may be local or remote with respect to a user. More than one machine in one location or multiple locations may be accessed by a user, and data may be mapped and/or processed in series and/or in parallel. Thus, a suitable configuration and control may be utilized for mapping and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (Ethernet/WiFi), a communication port (e.g., a USB port, HDMI port), Bluetooth, a PCMCIA slot and/or card, and the like. Data may be input by a suitable communication interface, device and/or method, including, but not limited to, manual input devices and/or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

In certain embodiments, simulated data is generated by an in silico process and the simulated data serves as data that can be input via an input device. The term “in silico” refers to data (e.g., contigs, intermediate contigs, supercontigs and the like), and/or a manipulation or a transformation of data that is performed using a computer, one or more computer program components, or a combination thereof. In certain embodiments methods and processes herein are performed in silico. In silico processes include, but are not limited to, mapping reads, aligning reads, overlapping reads, generating a pile-up relationship, iterative processes (e.g., iterative assembly or construction of contigs, intermediate contigs and/or supercontigs, or portions thereof), assembling haplotypes, assembling genotypes and/or genotype hypothesis.

A system may include software useful for performing a process described herein, and software can include one or more computer program components for performing such processes. The term “software” refers to computer-readable storage medium comprising program instructions (e.g., an executable program) that, when executed by a computer, perform computer operations. Instructions executable by the one or more microprocessors sometimes are provided as executable code, that when executed, can cause one or more microprocessors to implement a method described herein.

A computer program component (i.e., component) described herein can exist as software, and/or instructions (e.g., processes, routines, subroutines) embodied in the software which can be implemented or performed by a processor or microprocessor. For example, a computer program component can be a part of a program that performs a particular process or task. The term “computer program component” and “component” are used synonymously herein and refer to a self-contained functional unit that can be used in a larger machine or software system. A component can comprise a set of instructions for carrying out a function of the computer program component by one or more microprocessors. Instructions of a computer program component can be implemented in a computing environment by use of a suitable programming language, suitable software, and/or code written in a suitable language (e.g., a computer programming language known in the art) and/or operating system, non-limiting examples of which include UNIX, Linux, oracle, windows, Ubuntu, ActionScript, C, C++, C#, Haskell, Java, JavaScript, Objective-C, Perl, Python, Ruby, Smalltalk, SQL, Visual Basic, COBOL, Fortran, UML, HTML (e.g., with PHP), PGP, G, R, S, the like or combinations thereof.

In some embodiments a computer program component comprises one or more data files and can transfer data files to another computer program component and/or receive data files from another computer program component. In some embodiments a component transforms data and/or information, for example, into tangible printed matter, instructions to a user, an outcome, a display, a genotype, the like or combinations thereof. For example, one or more components and/or microprocessors (e.g., apparatus or machines) described herein can obtain sequencing reads, which represent random, unordered, nucleic acid fragments of a subjects genome, and transform those reads into an accurate representation (e.g., a display) of a specific portion of subject's body (e.g., a portion of a subject's genome (e.g., a genotype of a genomic region of interest)). The process can be compared to a process of transforming millions of pieces of a puzzle into a picture or transforming bits of X-ray data into a display of a portion of a subjects body (e.g., a display of bones, organs, and other body tissues).

One or more components can be utilized in a method described herein, non-limiting examples of which include a sequence component, a recruiting component, a pile-up relationship component, a supercontiger component, a contig assembly component, a supercontig assembly component, an insert size distribution component, an adapter trimmer component, a read-read aligner, a haplotype component, a caller, an outcome component, the like or combination thereof. Components are sometimes controlled by a microprocessor. In certain embodiments a component or a machine comprising one or more components, gather, assemble, receive, obtain, access, recover provide and/or transfer data and/or information to or from another component, machine, interface, peripheral or operator (user) of a machine. In some embodiments, data and/or information (e.g., sequence reads) are provided to a component by a machine comprising one or more of the following: one or more flow cells, a camera, a detector (e.g., a photo detector, a photo cell, an electrical detector (e.g., an amplitude modulation detector, a frequency and phase modulation detector, a phase-locked loop detector), a counter, a sensor (e.g., a sensor of pressure, temperature, volume, flow, weight), a fluid handling device, a data input device (e.g., a keyboard, mouse, scanner, voice recognition software and a microphone, stylus, or the like), a printer, a display (e.g., an LED, LCT or CRT), the like or combinations thereof. For example, sometimes an operator of a machine or apparatus provides a constant, a threshold value, a formula or a predetermined value to a component. A computer program component is often configured to transfer data and/or information to or from a microprocessor, a storage medium and/or memory. A component is often configured to transfer data and/or information to, or receive data and/or information from another suitable component or machine. A component can manipulate and/or transform data and/or information. Data and/or information derived from or transformed by a component can be transferred to another suitable machine and/or component. A machine comprising a computer program component can comprise at least one microprocessor. A machine comprising a component can include a microprocessor (e.g., one or more microprocessors) which microprocessor can perform and/or implement one or more instructions (e.g., processes, routines and/or subroutines) of a component. In some embodiments, a component operates with one or more external microprocessors (e.g., an internal or external network, server, storage device and/or storage network (e.g., a cloud)).

Data and/or information can be in a suitable form. For example, data and/or information can be digital or analogue. In certain embodiments, data and/or information sometimes can be packets, bytes, characters, or bits. In some embodiments, data and/or information can be any gathered, assembled or usable data or information. Non-limiting examples of data and/or information include a suitable media, pictures, video, sound (e.g. frequencies, audible or non-audible), numbers, constants, data files, a value, objects, time, functions, instructions, maps, references, sequences, reads, mapped reads, levels, ranges, thresholds, signals, displays, representations, or transformations thereof. A computer program component can accept or receive data and/or information, transform the data and/or information into a second form, and provide or transfer the second form of information to a machine, peripheral, device, microprocessor, storage device, interface or to another computer program component. A microprocessor can, in certain embodiments, carry out the instructions in a component. In some embodiments, one or more microprocessors are required to carry out instructions in a computer program component or group of computer program components. A computer program component can provide data and/or information to another computer program component, machine or source and can receive data and/or information from another computer program component, machine or source.

A computer program product sometimes is embodied on a non-transitory computer-readable medium, and sometimes is tangibly embodied on a non-transitory computer-readable medium. In certain embodiments a computer-readable storage medium comprises an executable program stored thereon. A computer program component sometimes is stored on a non-transitory computer readable medium (e.g., disk, drive) or in memory (e.g., random access memory). A computer program component and microprocessor capable of implementing instructions from a computer program component can be located in a machine or in a different machine. A computer program component and/or microprocessor capable of implementing an instruction for a computer program component can be located in the same location as a user (e.g., local network) or in a different location from a user (e.g., remote network, cloud system). In embodiments in which a method is carried out in conjunction with two or more computer program components, the computer program components can be located in the same machine, one or more computer program components can be located in different machine in the same physical location, and one or more computer program components may be located in different machines in different physical locations.

In certain embodiments, a machine, apparatus or computer comprises one or more computer component parts, peripherals and/or interfaces. Peripherals and/or computer component parts can sometimes transfer data and/or information to and from computer program components, interfaces, displays, peripherals and/or other computer component parts. In certain embodiments a machine interacts with a peripheral and/or computer component part that provides data and/or information. In certain embodiments peripherals and computer component parts assist a machine in carrying out a function or interact directly with a computer program component. Non-limiting examples of peripherals and/or computer component parts include a suitable computer peripheral, I/O or storage method or device including but not limited to scanners, printers, displays (e.g., monitors, LED, LCT or CRTs), cameras, microphones, pads (e.g., ipads, tablets), touch screens, smart phones, mobile phones, USB I/O devices, electronic storage (USB mass storage devices, optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media), keyboards, a computer mouse, digital pens, modems, hard drives, jump drives, flash drives, a microprocessor, a server, CDs, DVDs, graphic cards, specialized I/O devices (e.g., sequencers, photo cells, photo multiplier tubes, optical readers, sensors, etc.), network interface controllers, read-only memory (ROM), random-access memory (RAM), wireless transfer devices (Bluetooth devices, WiFi devices, and the like,), the world wide web (www), the internet, a computer and/or another computer program component.

Computer Program Components and Computer Implementation

In some embodiments a system comprises a sequence component that is configured to generate sequence reads. A sequence component may comprise a nucleic acid sequencer (e.g., a machine or apparatus designed and configured to generate sequence reads for a nucleic acid library) and/or software and instructions configured to generate, assemble, map, and trim sequence reads. A sequence component often provides sequence reads in the form of a data file (e.g., a bam file, a fasta file, and the like). A sequence component can provide sequence reads in any suitable file format.

In some embodiments a system comprises a recruiting component. In some embodiments a recruiting component is configured to obtain sequence reads (e.g., paired-end sequence reads), in the form of data files, from a suitable source and/or input means. For example, a recruiting component can obtain or receive reads from an apparatus configured to produce reads (e.g., an apparatus configured for nucleic acid sequencing) and/or from a computer configured to assemble and/or map reads. In some embodiments a recruiting component obtains and/or recruits reads from a sequence component. In some embodiments a recruiting component obtains reads from a suitable non-transitory or transitory storage medium. For example a person can provide sequence reads to a recruiting component by providing data files to a recruiting component by any suitable means (e.g., via a jump drive, disc, email, the internet, and the like). In certain embodiments a recruiting component obtains and/or recruits reads that are mapped to a reference and/or reads that are unmapped, discordant or poorly mapped to a reference (e.g., reads with low mappability). In some embodiments a recruiting component obtains read mates of pair-end sequence reads that are mapped and their corresponding read mates whether they are mapped, unmapped, discordant or poorly mapped. In certain embodiments a recruiting component obtains a set of paired-end sequence reads comprising a plurality of read mate pairs, each pair comprising two read mates, wherein at least one of the two read mates of each pair is mapped to at least one portion of a reference genome comprising a pre-selected genomic region of interest and wherein some of the paired-end sequence reads are not mapped to the at least one portion of the reference genome. In some embodiments a recruiting component obtains and/or stores information associated with a read (e.g., read length, orientation of read mate pairs and estimated insert length of read mate pairs). In some embodiments a recruiting component is configured to transferred selected reads (e.g., recruited reads, obtained reads, a selected set of reads) to another computer program component. For example in some embodiments a recruiting component transfers selected reads to a filter component, trimming component, mapping component, or pile-up relationship component, contig assembly component, supercontig assembly component and/or a caller component.

In some embodiments a system or storage media comprises an insert size distribution component. In some embodiments an insert size distribution component transfers and/or receives data and/or information from a recruiting component, supercontiger, supercontig assembly component, haplotype component or genotype likelihood ratio component. In some embodiments an insert size distribution component is often configured to determine an insert size distribution, an estimated insert size, estimated insert length, and/or estimated likelihood of insert size for a read pair or for a subset of paired-end reads. In some embodiments an insert size distribution component generates a distribution of estimated, calculated or measured insert fragments lengths and determines an estimated insert size for a subset of paired-end reads. An insert size distribution component sometimes incorporates or indexes an estimated insert size and/or estimated likelihood of insert size into a data file. In some embodiments an estimated likelihood of insert size is associated with a read mate pair and is used to determine how likely a given read mate pair maps or aligns to a contig or supercontig. In some embodiments an insert size distribution component determines likelihoods or probabilities associated with the mapping or alignment of a read mate pair to another read mate pair, a reference sequence, contig, or supercontig according to an estimated insert length.

In some embodiments an insert size distribution component assigns a likelihood (e.g., an insert size likelihood), likelihood score or penalty to a supercontig or genotype hypothesis. In some embodiments an insert size distribution component assigns a likelihood, likelihood score or penalty to a proposed insert size. For example, in some embodiments a haplotyper re-aligns a read pair with a supercontig or haplotype of a genotype hypothesis. In certain embodiments, an insert size distribution component is recruited by the haplotyper to determine if the distance between a re-aligned read pair, which distance is defined by the supercontig or haplotype, is consistent with an estimated insert size or insert size distribution for the read pair. In some embodiments an insert size distribution component compares (i) the distance between a re-aligned read pair, which distance is defined by the supercontig or haplotype to which the read pair is aligned, to (ii) an insert size distribution or estimated insert size determined for the read pair, and returns a likelihood, likelihood score or probability that, in some embodiments is associated with the supercontig or genotype hypothesis to which the read pair was aligned. For example, in some embodiments when read mates of a read mate pair map to a hypothesized sequence (e.g., a supercontig, haplotypes of a genotype hypothesis) and are mapped at a distance from each other that is longer than an estimated insert size, the likelihood of such a long insert size would be low and the low likelihood is used to penalize the likelihood ratio of that hypothesis. In some embodiments an insert distribution component determines a likelihood, likelihood score or probability according to a plurality of read pairs that are re-aligned to a supercontig or genotype hypothesis. In some embodiments an insert distribution component determines a likelihood, likelihood score or probability according to a plurality of read pairs that are re-aligned to a supercontig or genotype hypothesis and associates the likelihood, likelihood score or probability with the supercontig or genotype hypothesis to which the reads were re-aligned. In certain embodiments a likelihood or likelihood score comprises a penalty or penalty score that is associated with a supercontig, haplotype or genotype hypothesis. In certain embodiments, data and/or information (e.g., insert size distributions, estimated insert sizes, likelihoods, insert size likelihoods, likelihood scores, penalties or probabilities) is transferred to and/or is processed by a haplotype component to determine or pre-compute mapping weights for each read-pair to each haplotype. In some embodiments a mapping weight is determined, in part, according to one or more insert size likelihoods or penalties determined by an insert size distribution component. In some embodiments a mapping weight comprises an insert size likelihood, which is derived from an insert size distribution component.

In some embodiments a majority consensus nucleotide is determined according to a plurality of overlaps or alignments determined according to a pile-up relationship (e.g., as determined by a pile-up relationship component). In certain embodiments overlaps and/or alignments of reads are checked against an overlap and/or alignment of their corresponding read mates according to an estimated or implied insert length between the mapped read mates. Such a function is often performed by an insert size distribution component. For example, where two read mates of a read mate pair align with portions of a contig and the distance between the two read mate ends implies a certain insert size then the alignment will receive an insert size likelihood corresponding to the frequency of such insert size being generated by the lab DNA fragmentation protocol. In some embodiments, where two read mates of a read mate pair overlap or align with portions of a contig and the distance between the two read mates' ends, which implies their insert length, is quite frequent given the DNA fragmentation process, then such read-pair alignment is assigned relatively high likelihood. On the other hand a low likelihood would be assigned to a read-pair alignment that implies too short or too long insert size given the DNA fragmentation protocol. During a contig assembly or during assembly of a supercontig, overlap and alignment penalties are sometimes assessed. In some embodiments, overlaps and/or alignments that comprise a penalty are not included or used for assembly of a contig, intermediate contig or supercontig. Alignments and overlaps that include two read mates of a read mate pair are often checked by an insert size distribution component. In certain embodiments, an insert size distribution component assesses overlaps and alignments of read mate pairs to contigs, intermediate contigs, supercontigs and haplotypes according to insert lengths determined by the locations of the read mate ends. In certain embodiments, an insert size distribution component assigns weights and/or penalties or likelihoods to certain overlaps and alignments of read mate pairs (e.g., alignments of read mates to contigs, intermediate contigs, supercontigs and haplotypes). In some embodiments, an insert size distribution component determines a likelihood that a read mate pair (e.g., both read mates) was generated by an in silico generated sequence (e.g., a contig, supercontig, haplotype or haplotype hypothesis), wherein the likelihood is determined according to the insert length for the read pair implied by their alignments to the sequence. In some embodiments an insert distribution component associates a likelihood with each read pair alignment to an in silico generated sequence (e.g., a contig, supercontig, haplotype or haplotype hypothesis), wherein the likelihood is determined according to an insert size and the likelihood is included as the probability P(I_{M}) as shown in equations 3 and 6, where I_{M} is the insert size implied by mapping M of the read-pair. In some embodiments the probability P(I_{M}) can be obtained from an empirical insert-size distribution. An insert size distribution component often sends data and/or information to a pile-up relationship component, a contig assembly component, a supercontig assembly component, a caller and/or to a caller component.

In some embodiments a system comprises a pile-up relationship component (i.e., a relationship component). In some embodiments a pile-up relationship component determines one or more pile-up relationships. In some embodiments a pile-up relationship component is configured to perform alignments, generate overlaps and determine or assign relationships (e.g., pile-up relationships) to reads and/o read mates. In some embodiments a pile-up relationship component is configured to generate one or more pile-up relationships for a set of reads. A pile-up relationship component often obtains and/or receives reads from a recruiting component and generates one or more pile-up relationships according to the reads received. In certain embodiments, a pile-up relationship component generates all possible overlaps for a set or subset of reads. In certain embodiments, a pile-up relationship component generates overlaps for a set or subset of reads according to a suitable k-mer hashing strategy. In certain embodiments a pile-up relationship component filters, removes and/or prunes overlaps. In certain embodiments a pile-up relationship component selects and/or stores overlaps. In some embodiments a pile-up relationship component generates a pile-up graph and/or tiling chart. A pile-up relationship component often transfers selected overlaps and/or read-read alignments for a set of reads to a contig assembly component.

In some embodiments a system comprises a contig assembly component. In certain embodiments a contig assembly component receives data and/or information (e.g., data files) from a recruiting component or pile-up relationship component. A contig assembly component is often configured to assemble contigs by iteratively adding nucleotides (e.g., in silico) to a starter read or to an intermediate contig according to a pile-up relationship. A contig assembly component often determines overlaps and/or alignments of reads, read mates and/or read mate pairs to portions of a starter read, a contig or intermediate contig. In some embodiments a contig assembly component often determines overlaps and/or alignments of reads, read mates and/or read mate pairs according to pile-up relationship. In some embodiments a contig assembly component often determines overlaps and/or alignments of reads, read mates and/or read mate pairs according to penalties and/or weights determined for certain overlaps and/or alignments of reads. Penalties, weights and/or the absence thereof are often determined by an insert size distribution component and are sent to a contig assembly component where the information is used to include or exclude certain read overlaps or alignments during assembly of a contig. Contigs (e.g., contigs and intermediate contigs) generated by a contig assembly component are often transferred to a supercontig assembly component.

In some embodiments a system comprises a supercontig assembly component. In certain embodiments a supercontig assembly component receives data and/or information (e.g., data files) from a contig assembly component, a relationship component, an insert size distribution component and/or from a recruiting component. A supercontig assembly component is often configured to construct and assemble supercontigs by bridging contigs with one or more read mate pairs. A supercontig assembly component often determines overlaps and/or alignments of reads, read mates and/or read mate pairs to portions of one or more contigs or intermediate contigs. In some embodiments a supercontig assembly component often determines overlaps and/or alignments of reads, read mates and/or read mate pairs that connect two or more contigs. In some embodiments a supercontig assembly component often determines overlaps and/or alignments of reads, read mates and/or read mate pairs according to penalties and/or weights determined for certain overlaps and/or alignments of reads and read mate pairs. Penalties, weights and/or the absence thereof are often determined by an insert size distribution component and are sent to a supercontig assembly component where the information is used to include or exclude certain read overlaps or alignments during assembly of a supercontig. In some embodiments supercontigs generated by a supercontig assembly component are transferred to a caller or to a caller component. In some embodiments supercontigs generated by a supercontig assembly component are transferred to a haplotype component.

In some embodiments a system comprises a haplotyper (e.g., haplotype component) which carries out one or more haplotyper processes. One or more haplotyper processes are often performed by a haplotype component. A haplotype component may receive and/or exchange objects, data and/or information with one or more of a supercontiger component, a supercontig assembly component, pile-up relationship component, insert size distribution component, or recruiting component. A haplotype component can send objects, data and/or information to a caller or outcome component. In some embodiments a system does not comprise a haplotype component.

In some embodiments a system comprises a caller (e.g., a caller component). In certain embodiments a caller component receives data and/or information (e.g., data files) from a supercontig assembly component, a haplotype component, a relationship component, an insert size distribution component and/or from a recruiting component. In certain embodiments a caller assembles all possible genotypes for a given ploidy. In some embodiments a caller performs the functions of equation 6 (Eq. 6) and/or equation 1 (Eq. 1). In some embodiments a caller pre-computes the read pair weights for each read-pair and each allele (supercontig) and recalls the values during the hypothesis likelihood computation, which can be performed by the caller component. In some embodiments, to facilitate the computation of the read weights for all reads, the caller realigns all reads to all supercontigs. In some embodiments a caller maps all the reads to a reference genome or picks one of the haplotypes (e.g., sequence hypotheses) as a reference. In certain embodiments the first hypothesis determined becomes the reference and all likelihoods are computed relative to the first hypothesis.

In some embodiments a caller component generates one or more genotype sequences from one or more supercontigs according to ploidy (e.g., input ploidy, default ploidy). A caller can assemble genotype sequences (e.g., genotype possibilities, genotype hypotheses) according to any suitable ploidy. Genotype sequences can be pairs of supercontigs, in some embodiments, where ploidy is diploid. Genotype sequences can be single supercontigs were ploidy is haploid. Genotype sequences may consist of three supercontigs where ploidy is triploid. In some embodiments a caller assembles a plurality of genotype sequences from representing every possible genotype hypothesis for a given ploidy. Genotype sequences (e.g., genotype hypotheses) are often transferred from a caller to an outcome component.

In certain embodiments a caller receives data and/or information (e.g., data files) from a supercontig assembly component, a pile-up relationship component, an insert size distribution component and/or from a recruiting component. In some embodiments a caller generates one or more genotype sequence likelihood ratios for one or more supercontigs. In some embodiments a caller component generates a plurality of genotype likelihood ratios, where each likelihood ratio is generated for an assembly of haplotypes (e.g., a genotype hypothesis). Genotype sequence likelihood ratios that are generated by a caller component are often transferred to an outcome component.

In some embodiments a system comprises an outcome component. An outcome component often receives data and/or information (e.g., genotype probabilities) from a caller component. In some embodiments, an outcome component often obtains one or more genotype likelihood ratios from a caller component. Often an outcome is provided by an outcome component. An outcome sometimes is provided to a health care professional (e.g., laboratory technician or manager; physician or assistant) from an outcome component. An outcome component may comprise a suitable statistical software package. In certain embodiments an outcome component generates a plot, table, chart or graph. In some embodiments an outcome component generates and/or compares standard scores (e.g., Z-scores). The presence or absence of a genetic variation and/or associated medical condition (e.g., an outcome) is often determined by and/or provided by an outcome component. The presence or absence of a genetic variation in a subject is, in some embodiments, identified by a machine comprising an outcome component. An outcome component can be specialized for determining a specific genetic variation (e.g., an STR, translocation, polymorphism, insertion). For example, an outcome component that identifies an STR can be different than and/or distinct from an outcome component that identifies a translocation. In some embodiments, an outcome component or a machine comprising an outcome component is required to identify a genetic variation or an outcome determinative of a genetic variation by aligning the genotype sequences to the reference sequence. In certain embodiments an outcome is transferred from an outcome component to a display component where an outcome is provided by the display component (e.g., a suitable display, e.g., an LED or the like). In some embodiments an outcome component provides a representation of a genotype (e.g., a genotype sequence, a genotype image) to a display.

Genetic Variations and Medical Conditions

In some embodiments a system, process or method described herein determines the presence or absence of a genetic variation in a subject. In some embodiments the presence or absence of genetic alteration in a subject is determined according to a genotype likelihood ratio and/or an outcome component. A genetic variation generally is a particular genetic phenotype present in certain individuals. In some embodiments, a genetic variation is a chromosome abnormality (e.g., loss or gain of one or more portions of a chromosome). Non-limiting examples of genetic variations include one or more deletions, duplications, insertions, microinsertions, additions, translocations, mutations, polymorphisms (e.g., single-nucleotide polymorphisms, multiple nucleotide polymorphisms), fusions, repeats (e.g., short tandem repeats (i.e., STRs)), the like and combinations thereof. An insertion, repeat, deletion, duplication, mutation or polymorphism can be of any length, and in some embodiments, is about 1 base or base pair (bp) to about 250 megabases (Mb) in length. In some embodiments, an insertion, repeat, STR, deletion, duplication, mutation or polymorphism is about 1 nucleotide (nt) to about 50,000 nt in length (e.g., about 1 to about 10,000 nucleotides, about 1 to about 10,000 nucleotides, about 1 to about 10,000 nucleotides, about 1 to about 1,000 nucleotides, about 1 to about 500 nucleotides, about 1 to about 400 nucleotides, about 1 to about 300 nucleotides, about 1 to about 200 nucleotides, about 1 to about 100 nucleotides, or about 1 to about 50 nucleotides). In some embodiments a genetic variation that is determined by a process, system or method described herein consist of a length of about 2 to about 500 nucleotides, about 2 to about 400 nucleotides, about 2 to about 300 nucleotides, about 2 to about 200 nucleotides, about 2 to about 100 nucleotides, about 2 to about 50 nucleotides, 10 to about 500 nucleotides, about 10 to about 400 nucleotides, about 10 to about 300 nucleotides, about 10 to about 200 nucleotides, about 10 to about 100 nucleotides, about 10 to about 50 nucleotides, 20 to about 500 nucleotides, about 20 to about 400 nucleotides, about 20 to about 300 nucleotides, about 20 to about 200 nucleotides, about 20 to about 100 nucleotides, or about 20 to about 50 nucleotides in length.

A genetic variation can be comprised within a gene. A gene that comprises a genetic variation may include a genetic variation in or near the gene, which genetic variation may be in an intron, exon, untranslated region of a gene, or in a combination thereof. Any gene may comprise a genetic variation that is determined by a method or process described herein. For example, a genetic variation may be comprised with an AR, ATXN1, ATXNNX2, ATXN3, ATXN7, ATXN8, ATXN10, DMPK, FXN, JPH3, CACNA1A, PPP2R2B, TBP, ATN1, ARX, PHOX2B, PABPN1, ATT, CFTR and BRACA1 gene.

In certain embodiments a genetic variation, for which the presence or absence is identified for a subject, is sometimes associated with a medical condition. Non-limiting examples of medical conditions include those associated with intellectual disability (e.g., Down Syndrome), aberrant cell-proliferation (e.g., cancer), Non-Hodgkin's lymphoma, myelodysplastic syndrome, William's syndrome, Langer-Giedon syndrome, Alfi's syndrome, Rethore syndrome, Jacobsen Syndrome, retinoblastoma, Smith-Magenis, Edwards Syndrome, papillary renal cell carcinomas, DiGeorge syndrome, Angelman syndrome, Cat-Eye Syndrome, Familial Adenomatous Polyposis, Miller-Dieker syndrome, presence of a micro-organism nucleic acid (e.g., virus, bacterium, fungus, yeast), and preeclampsia.

### EXAMPLES

The examples set forth below illustrate certain embodiments and do not limit the technology.

### Example 1: Kragle: Local De-Novo Assembler and Genotype Caller for Short Tandem Repeat Sequences and Other Complex Loci

Kragle was designed as a local de-novo sequence assembly and genotyping package. Kragle was designed to assemble any ploidy sequence from paired-end reads. Kragle was specifically designed to handle repeat sequences extending up to read length, but can also call sequence junctions resulting from sequence inversions, translocations, duplications or deletions. Kragle was successfully applied to call diploid genotypes of short tandem repeats (STR's) in AR, ATXN1, ATNX2, ATXN3, ATXN7, DMPK, FXN, and HTT genes that were implicated in an array of genetic conditions. Kragle was also used to confirm a hypothesized junction resulting from a large deletion in the human BRACA1 gene, as well as to call complex variations involving homopolymer and adjacent di-nucleotide repeat in a human CFTR gene.

The functionality of Kragle is divided into four main components (FIG. 1): a read recruiting component (i.e., recruiter, recruiting component), a supercontiger, haplotype creator (haplotyper), and genotype hypotheses caller (caller). The supercontiger includes three additional components, a pile-up relationship component, a contig assembly component and a supercontig assembly component. The first two main components (recruiter component and supercontiger) are designed to take full advantage of paired-end reads to assemble low-complexity sequences that may include uninterrupted repeat content as long as the read length, and sometimes even longer if a repeat contains interruptions. The haplotyper constructs haplotypes from supercontig sequences, remaps all reads to them and attempts to identify irregularities in the assembled sequences. The genotype probability component assembles haplotype sequences (e.g., supercontigs) from the supercontigs assembly component and generates genotype probabilities and assigns confidence values. The caller calls diploid genotypes from the assembled haplotype sequences and assigns confidence based on likelihood ratios to alternative genotype hypotheses. The Kragle embodiment in this example does not utilize any read error correction as it was determined that read error correction algorithms interfere with determining the presence or absence of repeats (e.g., STRs).

In some embodiments the inputs to Kragle are reference sorted bam file and an insert size distribution file. Alternatively, Kragle accepts two fastq files (for read 1 and read 2) and a reference fasta file to call adapter trimmer component, BWA-MEM aligner, insert size distribution component and samtools (reference-sort and index bam file) to create the necessary inputs in a pre-processing step (see FIG. 1).

Recruiting Component

Sequence reads obtained from genomic regions of a sample that significantly differ from a reference sequence, present significant challenges to standard read aligners. For example, reads that originated from an altered part of a genome were often mapped to a wrong genomic location or remain unmapped. In such cases, however, the read mate from a read mate pair often contains the sequence of the unchanged (or little changed) flanking region and therefore can be mapped correctly. In order to circumvent the read mapping problem in the areas of expanded STR's, sequence junctions and large complex variations, the recruiting component uses the locations of the mapped read mates from the read pairs to identify reads that could be informative of a region that Kragle is trying to assemble (e.g., a particular genomic region of interest).

In some embodiments, Kragle is configured to use reference-sorted bam files to quickly index into a region or regions of interest. In cases of junctions, Kragle uses two genomic regions, which might have been identified using split-read signal or discordant mate signal, to retrieve the informative reads. The recruiting component then collects reads that had at least one of the mates mapped with a primary alignment to the region(s) of interest.

Supercontiger

The supercontiger is comprised of three components: a pile-up relationship component (e.g., a read-read aligner), a contig assembly component, and a supercontig assembly component. The three components start with a set of recruited read-pairs obtained from the recruiting component and produce a set of haplotype sequences (supercontigs). Supercontigs are composed of one or more contigs linked together in the right orientation and which are ordered to produce a haplotype sequence (e.g., a possibly interrupted haplotype sequence) of the assembled genomic region of interest.

Two general paradigms were used for contig assembly: de Bruin graph based (Idury R M, et al.; Pevzner P A, et al.) and Overlap-Layout-Consensus that relies on an overlap graph (Myers E W, et al., (2005)). Kragle used a read tiling strategy similar to, but distinct from the process of building “unitigs” in the Celera assembler (Myers E W, et al., (2000)) and in-spirit similar to, but distinct from, the Overlap-Layout-Consensus strategy. In this example, all possible overlaps between reads are identified before the contig assembly starts.

Pile-Up Relationship Component

The pile-up relationship component is configured to perform the function of identifying such possible read-read overlaps and also can eliminate some redundancy in the constructed graph. In contrast with overlap graph (Myers E W, et al., (2005)), which eliminates each read that was fully contained in a sequence of another read, the read tiling graph produced by the pile-up relationship component contains all reads as vertices, and edges represent read-read overlaps. The advantage of performing only local de-novo assembly and recruiting read pairs by mapped mates is that the orientation (strand) of each read is known and the read tiling graph is not required to represent the two strand possibilities for each read. This simplifies the read tiling graph and the assembly task because alternative read orientations are not explored.

The read tiling graph construction is performed in two steps. The first step identifies all read-read overlaps that pass a score threshold. The second step prunes the edges in the tiling graph to keep only a minimal set of overlaps necessary for constructing a complete tiling of contigs.

A k-mer hashing strategy is used to speed-up the identification of read-read overlaps. Each read is decomposed to a set of all possible k-mers (e.g., default k-mer size is 50), and all read-read pairs are quickly screened for a matching set of k-mers. If a match is found, if the alignment score passes a predetermined score cutoff, and if the corresponding k-mer position do not imply any insertions or deletions (in/dels) in the read-read alignment, then the inferred overlap between two reads in the read tiling graph is stored. The alignment score is computed as a sum of match and mismatch scores customary in alignment software (Smith et al., (1981); *Smith et al., (1981); Needleman et al., (1970)). The full read-read alignment is computed only if the corresponding k-mer positions implied insertions or deletions in the alignment. However the penalty for initiating or extending insertions and deletions is set sufficiently high to prohibit them, so the resulting read-read alignment is always in/del free. Again, if the resulting glocal alignment score passes the cutoff, the overlap is stored in the read tiling graph.

Reads with in/del sequencing errors are usually excluded from the assembly as a consequence of not allowing in/dels in the read-read overlap alignments. Such errors are quite rare and do not significantly reduce the read coverage. However, in/del—free overlaps simplify and significantly accelerate contig assembly. Note that true in/del variants (relative to a reference sequence) are aligned properly among reads, because all reads originating from such haplotypes contain the variation and therefore their overlaps are aligned correctly.

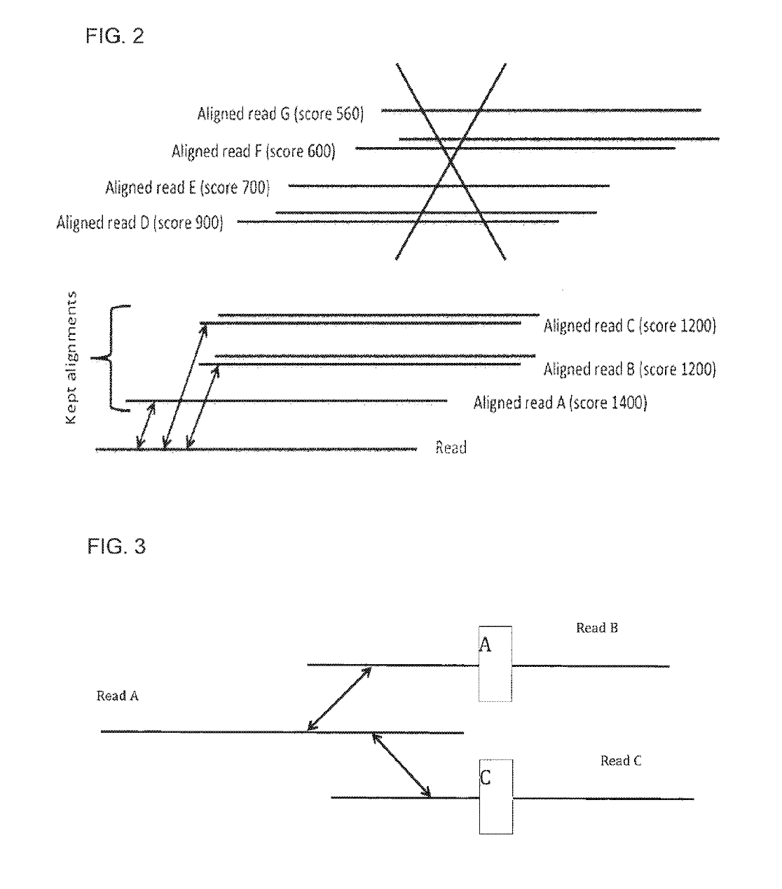

Once a full read tiling graph is constructed, it is pruned to eliminate unnecessary and possibly false overlaps. After the pruning only the best scoring overlap(s) and overlap(s) necessary to maintain connectivity in the graph are kept (e.g., stored) for each read. To maintain the graph connectivity each read keeps a minimum number (default one) of best scoring overlaps that extend it on each of the 3′ and 5′ sides and these connected reads however also must have a minimum number of their own overlaps extending it on the same side. For instance with a default minimum number of one connection, a read's connections is considered satisfied on the 3′ side if it has at least one overlap with another read that extends the read on the 3′ side and that extending read itself has at least one overlap with another read extending it also on the 3′ side (e.g., see FIG. 2). Then by implication the read maintains its connectivity in the graph and is reachable by overlap edges from the 3′ and 5′ sides, if it has such connections at the beginning of the process. Therefore any sequence supported by continuous read pileup with no coverage gaps maintains an uninterrupted path through the read connections.

The pruning algorithm iterates over all reads and maintains a list of overlaps for each read that are kept. At each iteration the process picks, for each read, the best scoring overlap (or overlaps, if there are more than one with the same score) that extends a read on the 3′ and 5′ sides, unless the read already satisfies the required minimum number of connections on each side (e.g., connected to reads on a side, which reads also have connections on the same side). The pruning iteration terminates when each read has a required minimum number of connections on each side. A read does not need to meet the required number of connections if it runs out of overlaps. Such reads are most likely reads at the end of contigs, or reads with too many sequencing errors to have any overlaps with other reads. After terminating the iteration the algorithm then deletes all overlap edges that are not in the list of edges to keep. It should be noted that an overlap edge is kept if either of the overlapping reads consider that connection necessary. For instance, if there are two reads spanning a polymorphism, each on the opposite haplotype, and a read that is just outside of the polymorphic position has overlaps with both of the polymorphic reads, then the read that is outside may keep an overlap with only one of the polymorphic reads to satisfy its connections, however the other polymorphic read may require a connection to that read to maintain its own connectivity and therefore the read that is outside of the polymorphism keeps both overlaps (FIG. 3). This ensures that reads from sequence stretches that are common to more than one haplotype are reached from all of the haplotype-specific reads.

Contig Assembly Component

The contig assembly component uses the read tiling graph to collect overlapping reads and extend the paths of overlapping reads through the graph. Each contig assembly is started from a single read. The contigs assembly first assembles two contigs that are initiated from a read picked from the 3′ side and the 5′ side of the region that the assembler was trying to call (e.g., the genomic region of interest). Each contig uses each read only once, however different contigs can share reads. The contig building process creates new contigs by splitting an existing contig upon encountering a polymorphic location. Once the two initial contigs and their split-off contigs are finished, the contig assembler inspects the set of reads that were not used in any of the contigs. If it finds among the unused reads a connected cluster of reads larger than a cutoff (computed as percentage of mean coverage depth from already assembled contigs—default 10%), then it starts building a new contig from one of the reads in the cluster. New contigs are started until there are no unused read clusters larger than the cutoff.

The contig builder keeps track of all reads that are used in the contig and splits them into two groups: reads that have already recruited their overlapping reads (using overlaps in the read tiling graph), and reads that have not recruited yet. The builder also maintains 3′ and 5′ “all recruited” boundaries (i.e., edges). The “all recruited” boundaries are set on the 3′ side just before the start of a first read that has not recruited yet and equivalently on the 5′ side. Therefore these boundaries set an interval (e.g., the advancing position) in the growing contig that does not change its read composition by newly recruited reads. Thus it is safe to compute a consensus sequence from the read pileup inside the “all recruited” interval.

Contig building progressed by repeating the following three steps:
(1) Recruit new reads using reads that have not recruited yet and overlap the position 1 base outside the current “all recruited” 3′ or 5′ boundaries (see FIG. 4). The recruited read must be either contained inside the recruiting read, or extend the read to the outside—away from the “all recruited” edge.
(2) Recompute “all recruited” boundary. The 3′ boundary may not have changed if any of the reads on that side of the boundary recruited a read that when placed in the contig starts at the same position. Similarly, the 5′ boundary may not have changed. However, also either of the two “all recruited” boundaries can shift by more than one base if newly placed reads leave a larger gap.
(3) Compute consensus sequence up to the new “all recruited” interval boundaries using the read pileup inside the “all recruited” interval.

The contig building iteration terminates when there are no reads that have not recruited yet. That happens if the contig building reaches the end of a region covered by the recruited reads (or reached a gap in coverage) and the reads on the edges do not have any overlapping reads extending the contig to either side.

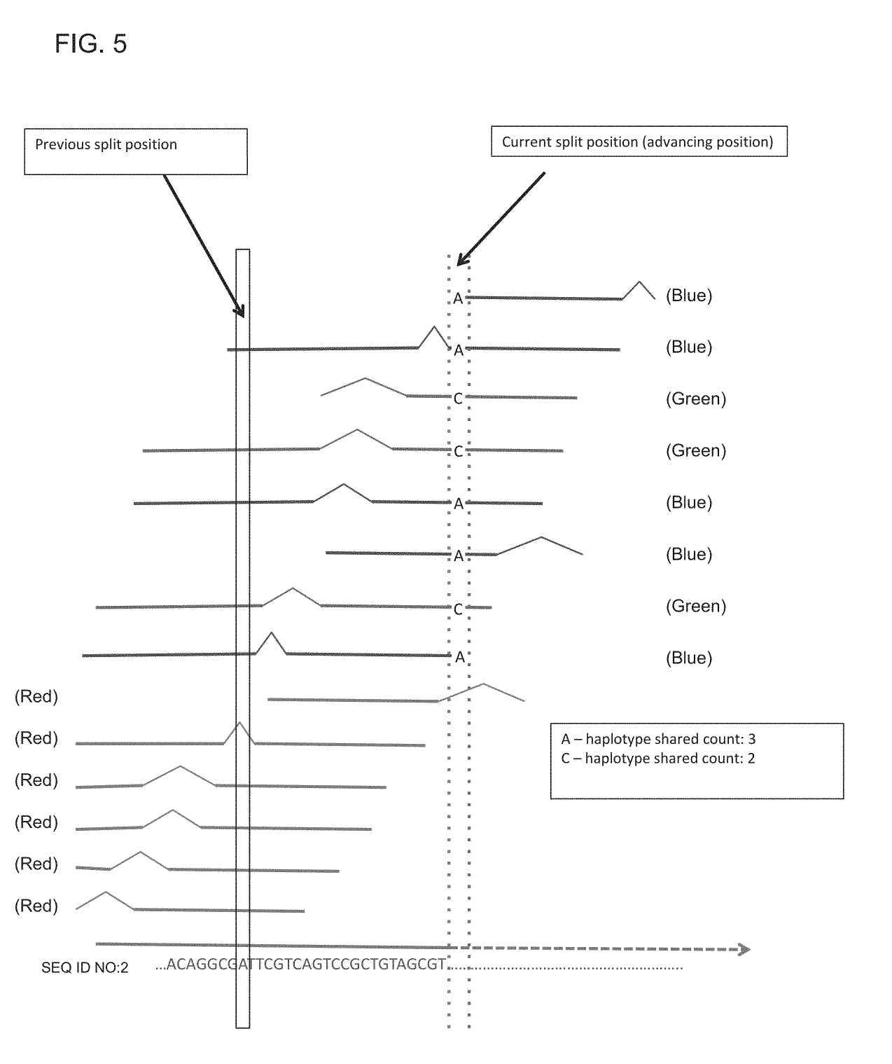

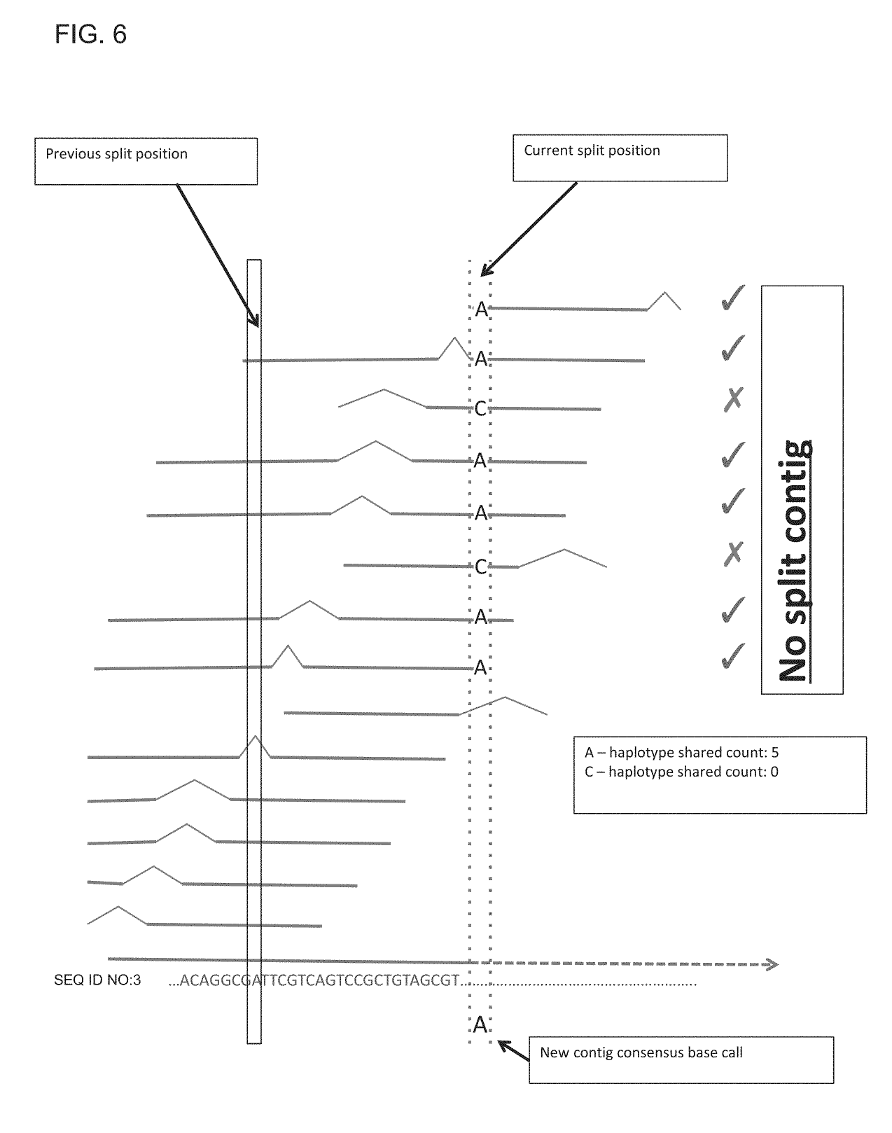

When the computation of the consensus sequence encounters a polymorphic position, i.e. position where the read pileup contains significant counts of two or more different bases, the current contig is split into two (or more) representing the two (or more) possible haplotypes. However, the count of a base is considered significant only if it exceeds 10% of the coverage at the position or at least 5 reads. Therefore random base call errors in reads will not likely trigger contig splitting. At this point the polymorphic reads are also checked for haplotype compliance. If the contig was already previously split at some position(s) the splitting process checks if the reads or their mates overlap the previously split position and collects the counts of reads that do (see FIG. 5). If read for one polymorphic base (or their mates) do overlap the position with a significant count (by default at least 5), but some other polymorphic base reads and their mates do not cross that position (count 0), then that polymorphic base will not be used for splitting another contig and these polymorphic reads and their mates will be removed from the contig (see FIG. 6). The rationale behind this design decision is to prevent splitting on a polymorphic position whose haplotype has already been segregated away in another contig. If a polymorphic position is sufficiently distant from previous polymorphic position that already split the contig into two (or more), and the read overlaps allow to recruit again some reads from the already split off haplotype, these reads will have no overlap (themselves or their mates) with the previous split position, because that position was already “purified” to contain the reads only belonging its haplotype. Therefore these reads (and their mates) will be just deleted from this contig, but will most likely be used in the contig that was already split off at the previous split position.

The process of contig splitting involves replicating the read membership in both contigs, except for the reads spanning the polymorphic position, where each contig will take the reads with their assigned base. The polymorphic reads and their mates are assigned to their respective contigs together. Since the mates reassignment can potentially eliminate read coverage in some sections of the “all recruited” interval (this is rare), we need to re-call the consensus sequence in each of the split contigs, assigning N's to regions with complete loss of coverage.

Since splitting off new contigs and building them is computationally expensive we employed several checks to reduce the geometric contig multiplication:

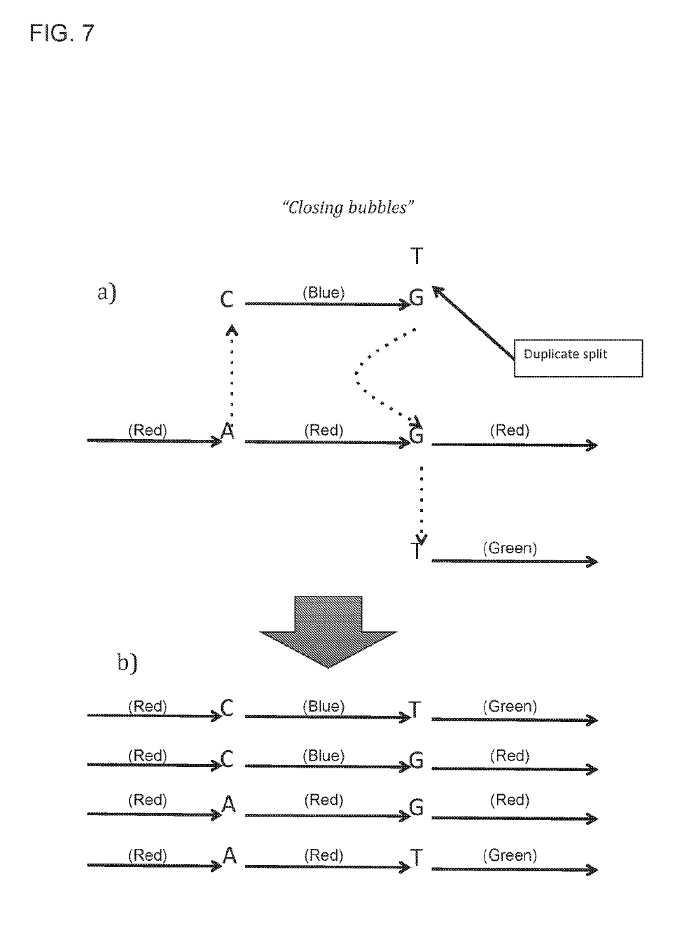

1. A split will not generate new contig(s) if that same split was already encountered in other contig (splitting with the same set of polymorphic reads and at the same positions in the reads). These split positions correspond to “closing haplotype bubbles” and these “not taken” splits with their corresponding “duplicates” are saved. These “not taken” paths are added later by concatenating all possible contig sequence endings to the contig after the “not taken” split. These endings are taken from the “duplicate” split contig and all contigs that it split off after this position (see FIG. 7). This criterion assumes that the potentially different read membership before the “duplicate” split is encountered would not result in different endings due to the constraint on read reuse. It assumes that once the same read pileup is encountered in the contig, the possible endings will be identical.
2. If the split off polymorphic reads cannot recruit any new reads to extend the split off contig, the contig is labeled with “dead end” and will not be used in supercontig construction. We observed that these splits happen when the contig encounters a set of reads with systematic basecall errors. These can be quite common especially in challenging areas, such as STR regions.
3. If graph cycle is detected by duplicating a split that was already taken in the contig (very rare), then the contig is labeled as “bad” and terminated. These contigs are also not used in supercontig construction.

The contigs assembled as described above are filtered for duplicates. These can result from the duplicate effort of assembling the same region starting with two reads—one from the 3′ and another from the 5′ side. However, the two starting points give the contig assembler better robustness against the shortcomings of the greedy read recruitment process (where a read is recruited by the first read in the contig that has an overlap with it in the read tiling graph). Exploring the paths through the graph from the two directions may result in some circumstances in somewhat different assembled sequences.

Supercontig Assembly Component

The contigs assembled in previous steps can either span the entire region that Kragle is trying to call, or they can terminate in places where coverage drops off or where high read error rates (usually systematic errors) prohibit high-scoring read-read overlaps. In such cases the read pairs can be used to link the contigs together to form supercontigs. If the gap between the reads in the read-pair allows them to be placed in the two neighboring contigs, straddling the coverage gap between the contigs, then such read-pairs can inform the contig link and its orientation.

The supercontig construction involves constructing a graph with contigs as vertices and the identified links as oriented edges. The oriented edges are recorded where a contig pair shares at least a minimum number of read pairs with the same orientation (minimum number set by default to 10% of mean contig coverage or at least 5 read pairs). The contigs are then connected to supercontigs by traversing all paths through the graph, while avoiding cycles, stating from all vertices with in-degree 0 and ending with vertices with out-degree 0 (see FIG. 8). Contigs that are not connected to any other contigs (have both in-degree and out-degree 0) create supercontigs with just one contig.

Haplotyper

Since the contig and the supercontig construction creates all possible sequence arrangements, the supercontigs represent possible haplotype sequences, which will be combined to generate correct ploidy (i.e. diploid) sequence hypotheses in the caller. The haplotyper initiates the haplotype objects and performs re-mapping of all reads to all the haplotype sequences. This re-mapping also includes the pre-computation of mapping weights described in the “Caller” section. The haplotyper also performs identification of false junctions and false insertions in the haplotype sequences. The main reason why the haplotyper is separated from the caller is to allow the calling program the opportunity to filter haplotypes, based on the outcomes of the identifications of false junctions and false insertions, before they are combined to sequence hypotheses. The identified false junctions and false insertions are listed in each haplotype object with necessary information determining their support by the reads. The calling program can use cutoffs on the supporting information to apply its own stringency of haplotype filtering.

False Junction Identification:

The algorithm aims to identify junctions between sequences that belong to different parts of the genome and were joined in the assembly process due to some sequence similarity. The sequence similarity would allow some reads to join the sequences, however the junction would be sparsely covered. Therefore these junctions can be identified as points in the sequence where the number of read-pairs straddling the junction location is much lower than expected. The algorithm finds possible false junctions by computing the expected number of mates some distance away (range of the insert size distribution) and compares them to the observed count. Locations of low observed/expected ratios are then marked as possible false junctions. One can use the chi-square test to determine the significance of the observed-expected difference, however such p-values would be increasingly sensitive with increasing coverage to locations with legitimate variability in the insert size distribution.

The algorithm uses an un-centered band around the mean of the insert size distribution to test for the false junctions. An interval of −20% and +80% of a band (currently size 50 for Illumina read-pair libraries) around the insert size distribution mean is used to compute the expected count and used to search for the observed count. The algorithm processes separately the observed and expected counts for forward reads and reverse reads (in the reverse direction) and then finds the local minima in the ratios. The algorithm reports all local minima that exceed specified ratio cutoff. Performing the search in both forward and reverse directions can give algorithmic confirmation of the junctions.

The algorithm however can yield false positives in some situations and therefore filtering of haplotypes with false insertions must be done with caution. The algorithm will yield false positives for large homozygous repeats that were compressed to read-length for both alleles. In such cases there will not be any reads that will span the compressed repeat sequence but the expected counts can still be large. The algorithm can also falsely flag positions that are highly erroneous (systematic and correlated errors in Illumina sequencing process). The highly erroneous sequence positions will have low coverage with mapped reads (reads with too many errors will not map) and thus may result in discrepancies between the expected and observed counts.

False Insertion Identification:

False insertion detection algorithm aims to detect insertions of foreign (or misplaced) sequences that were joined at the flanks with the parent sequence using some sequence similarity at the junctions. Such insertions could potentially be identified using pairing of false junctions (described above), however the dedicated algorithm described below is much more sensitive and specific than the false junction algorithm.

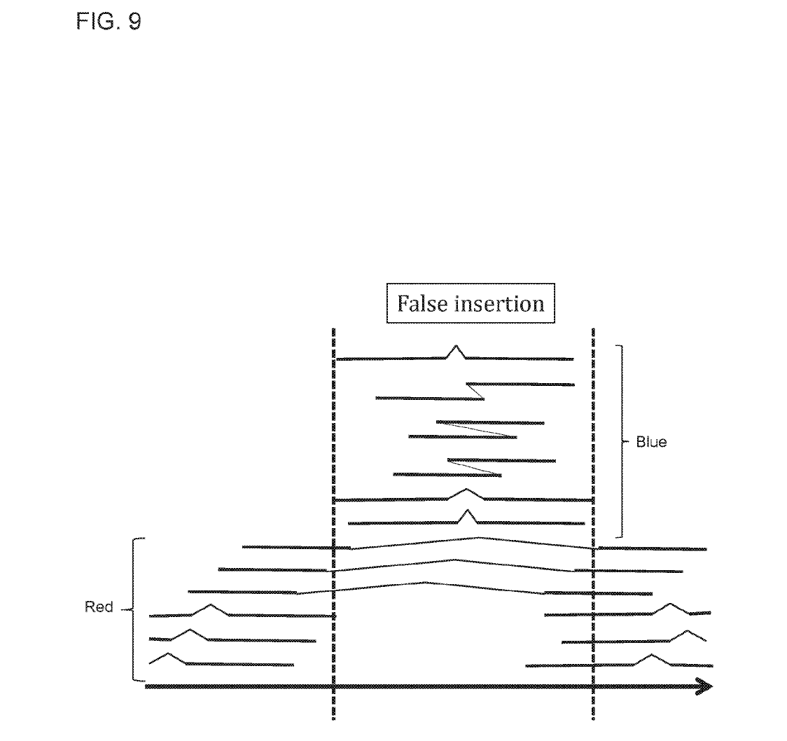

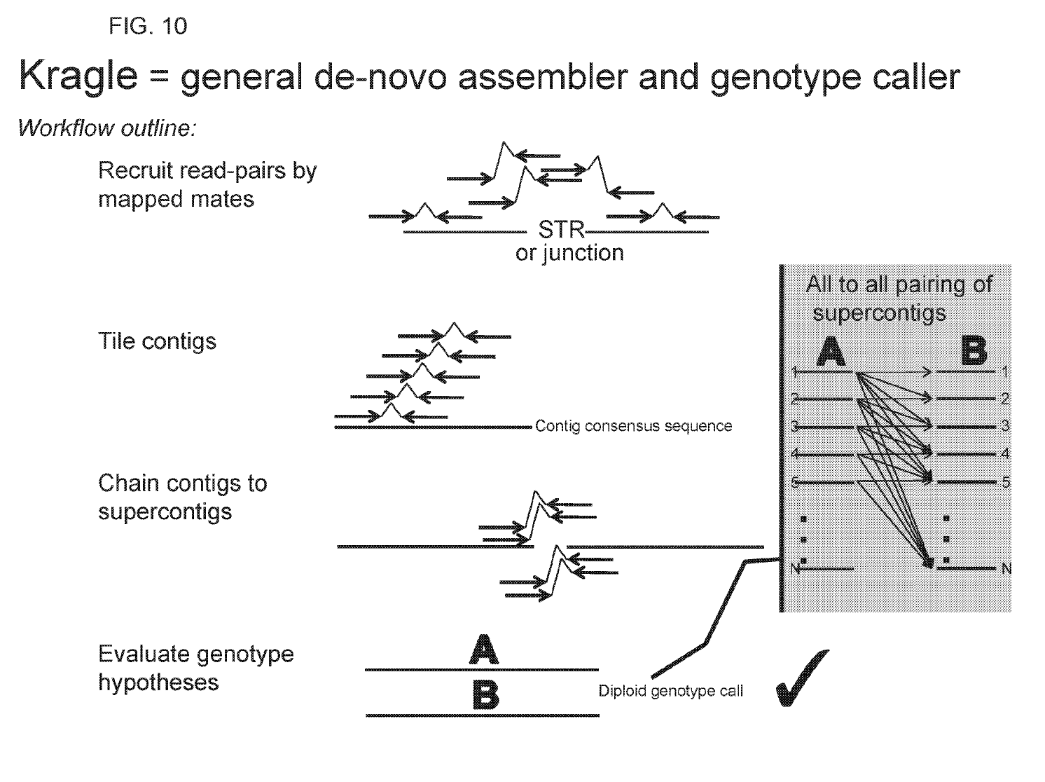

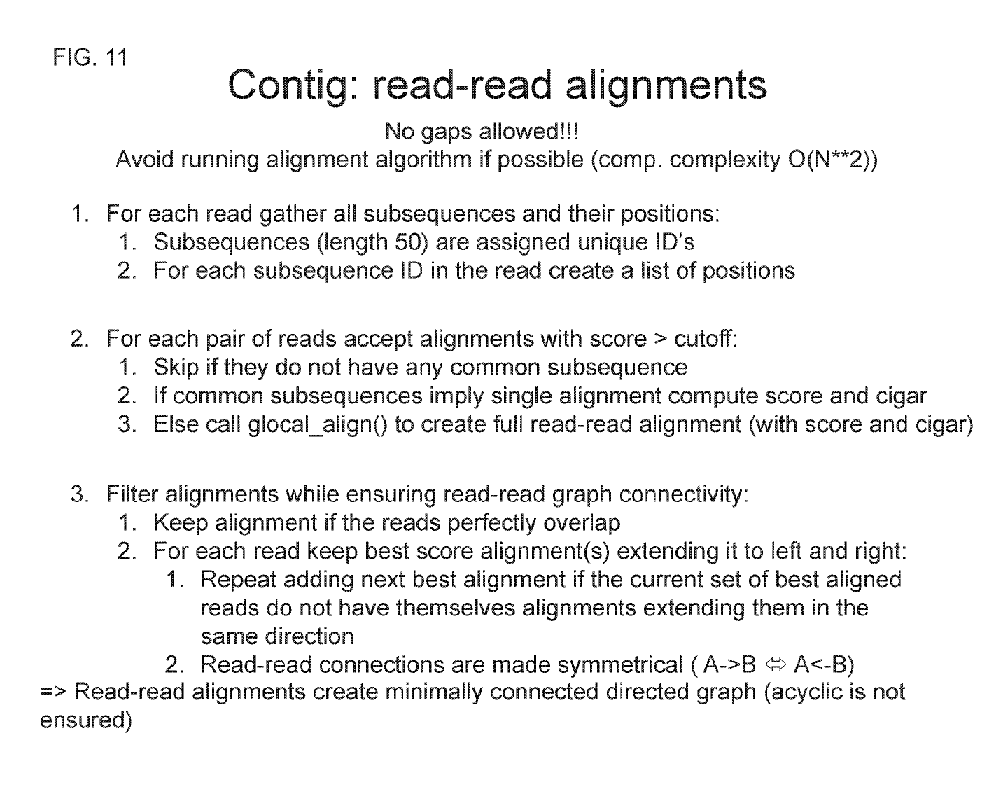

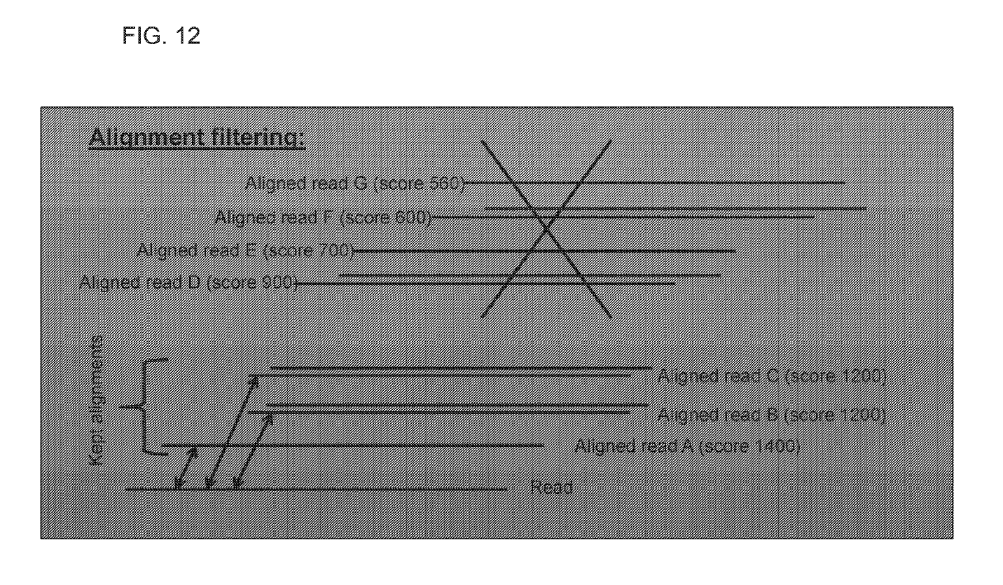

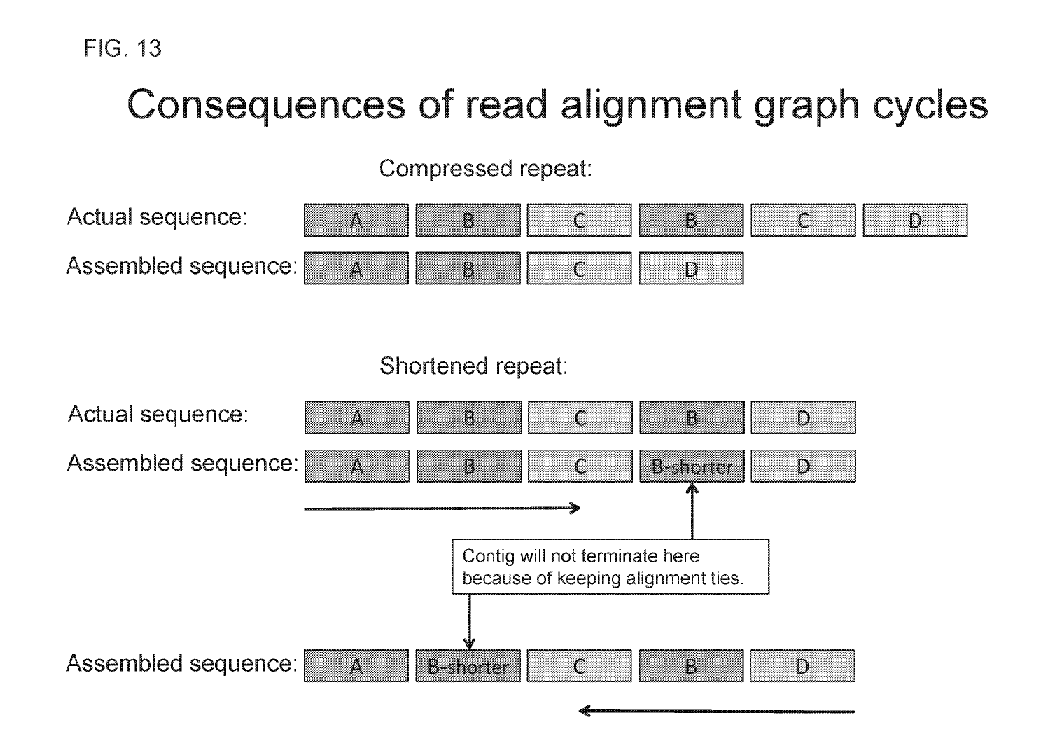

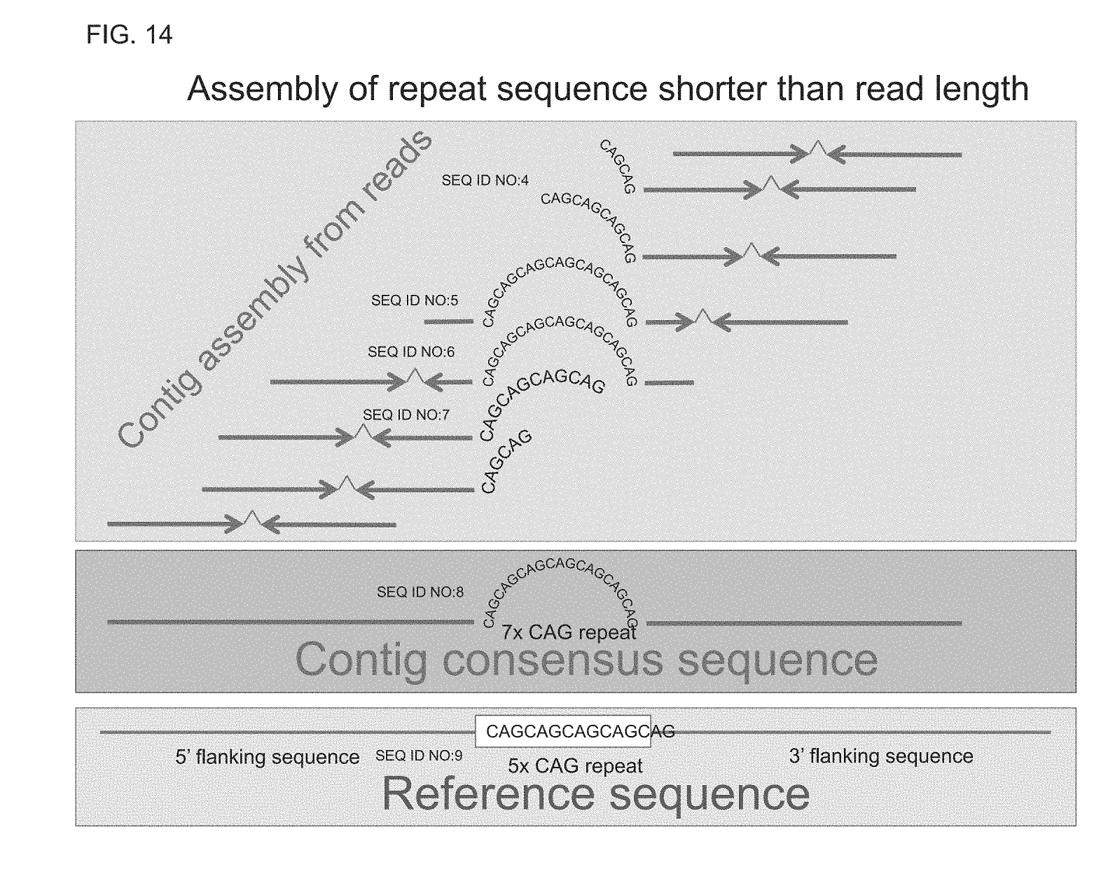

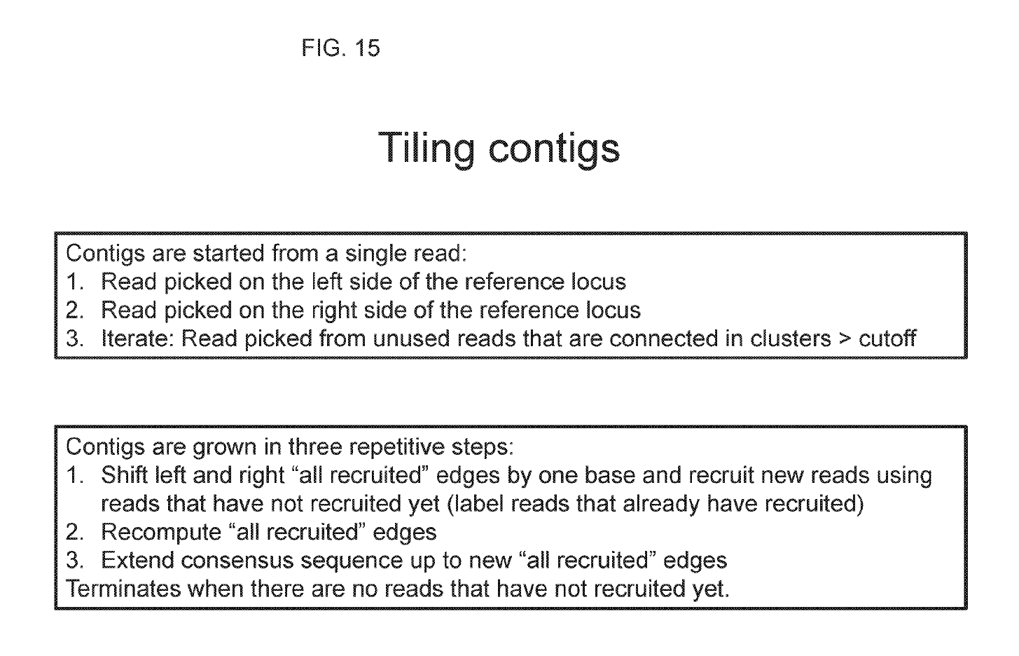

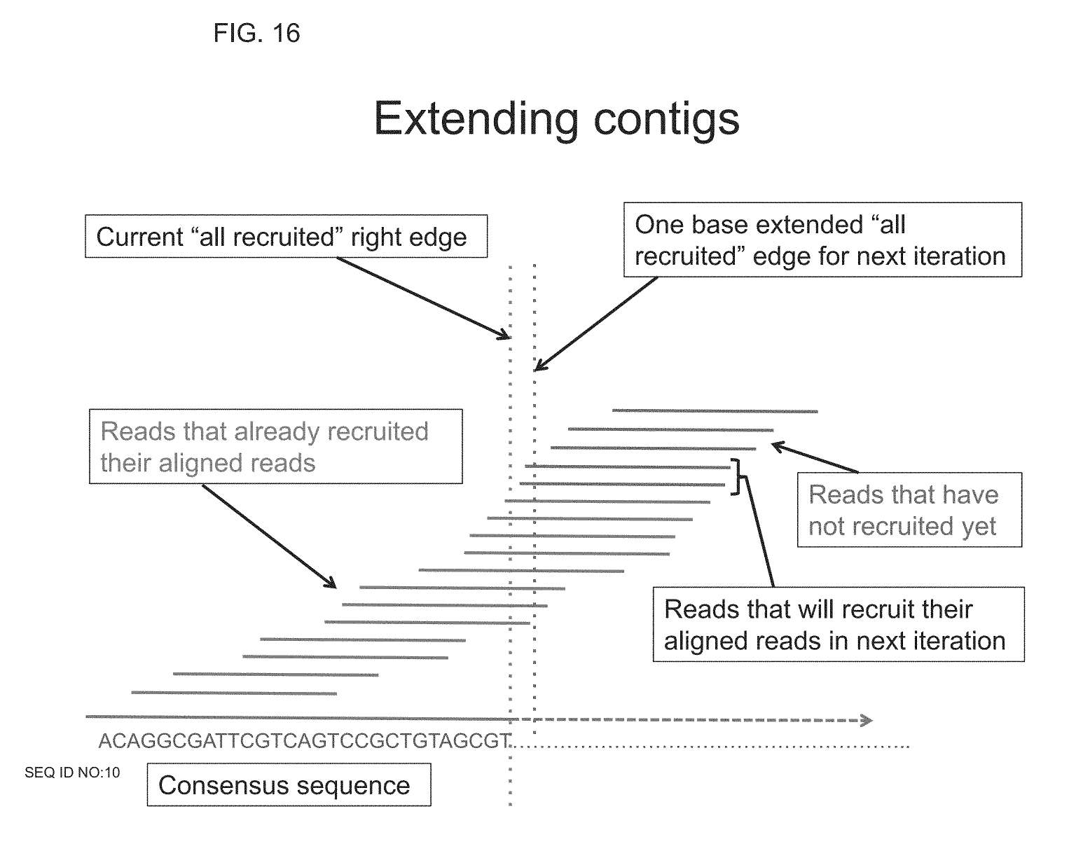

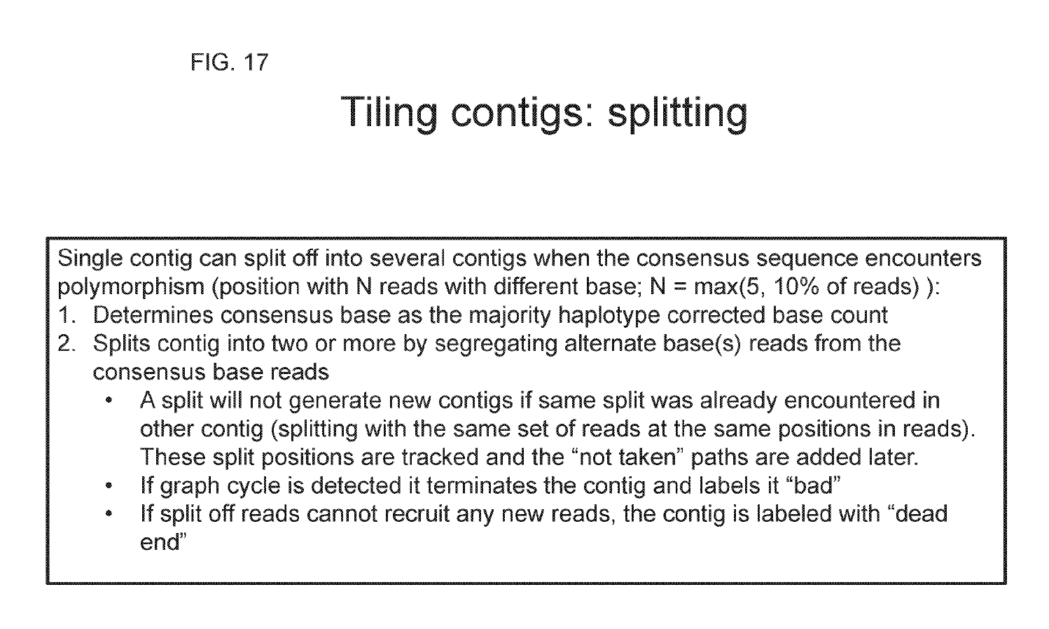

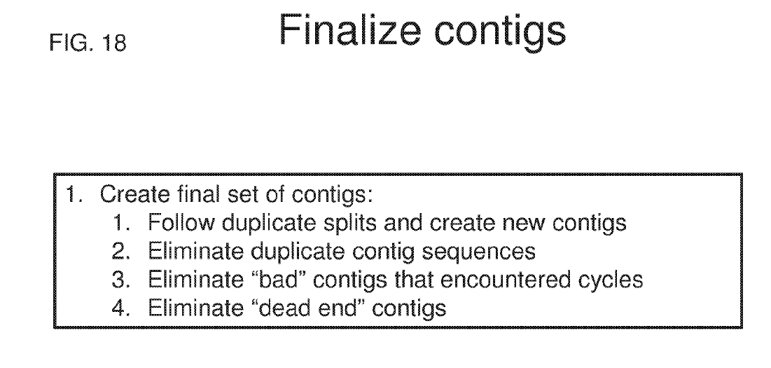

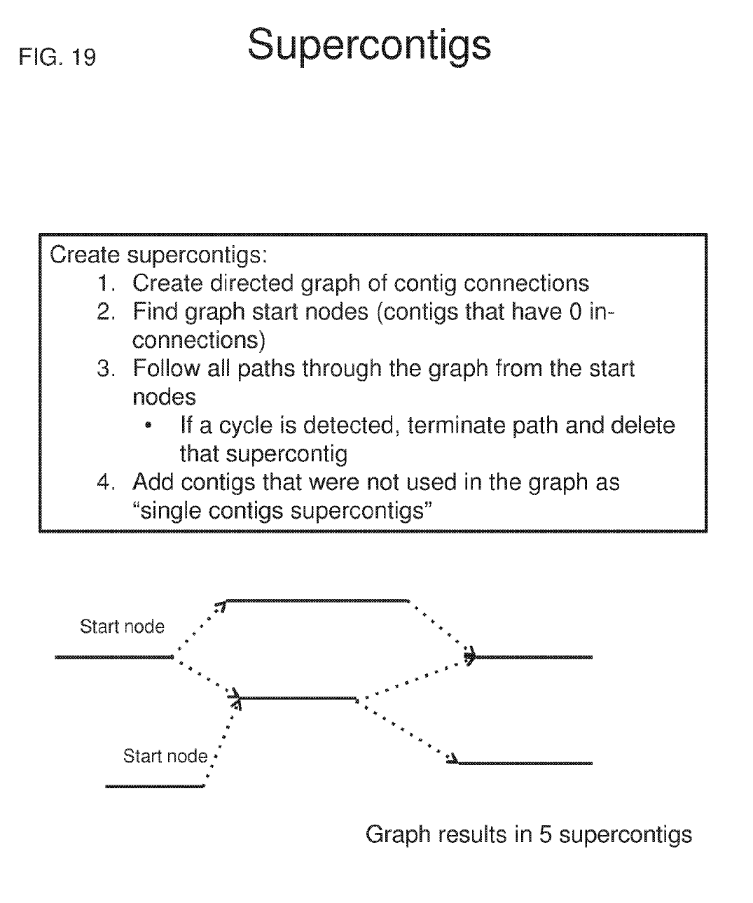

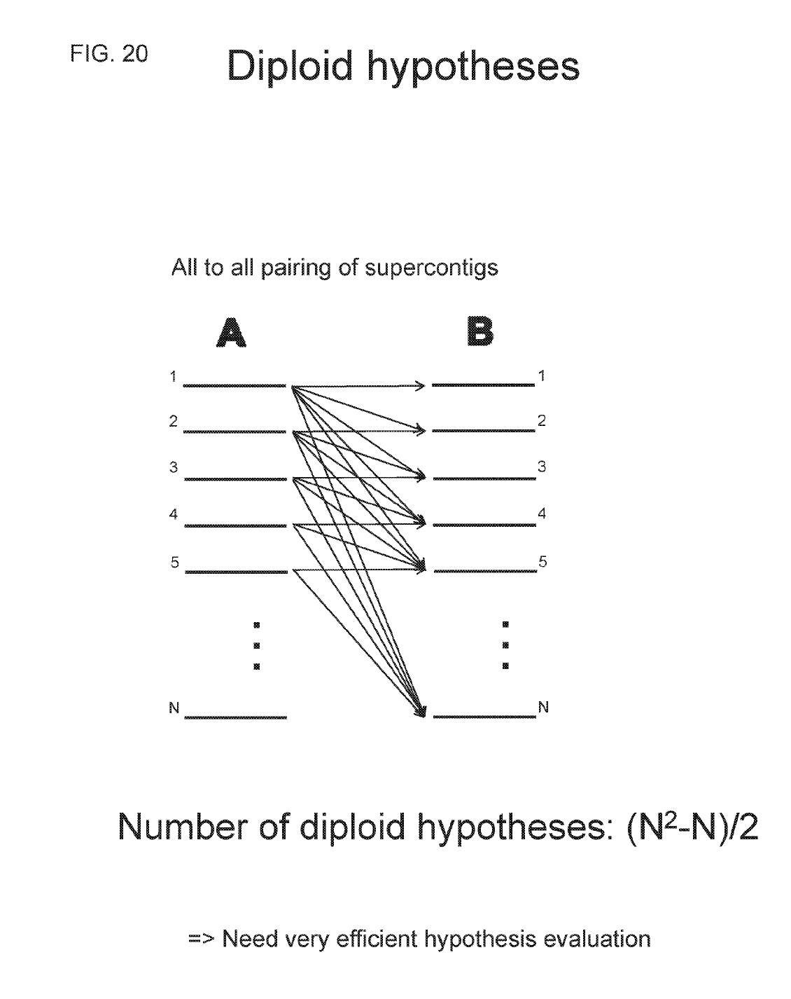

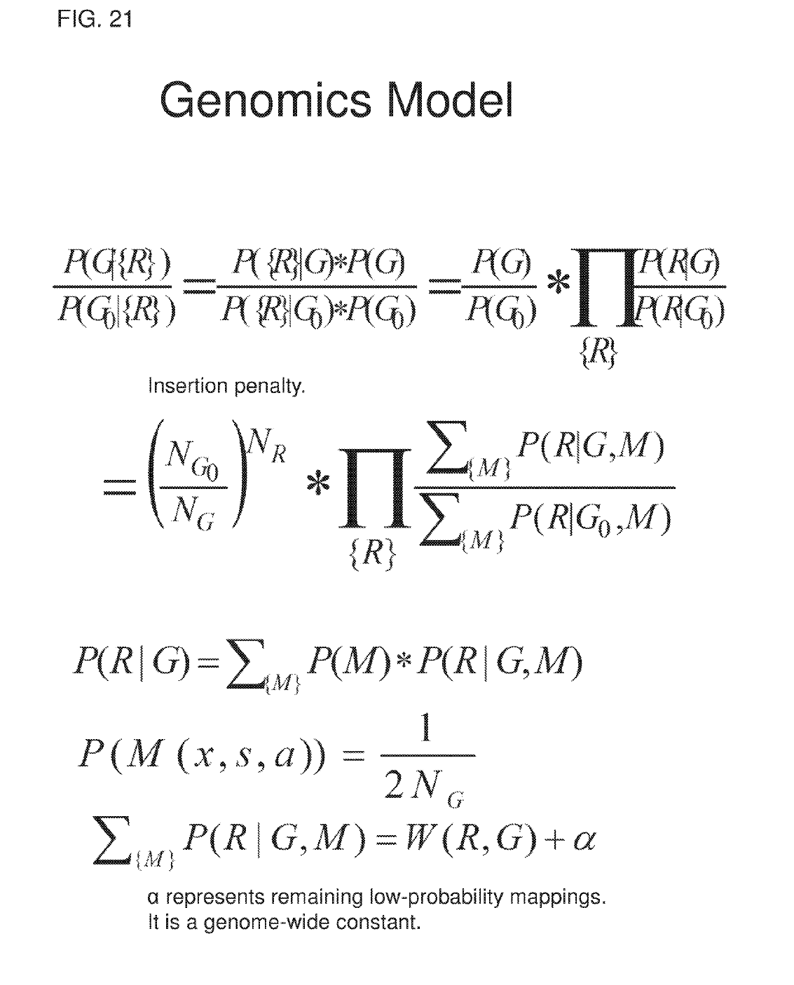

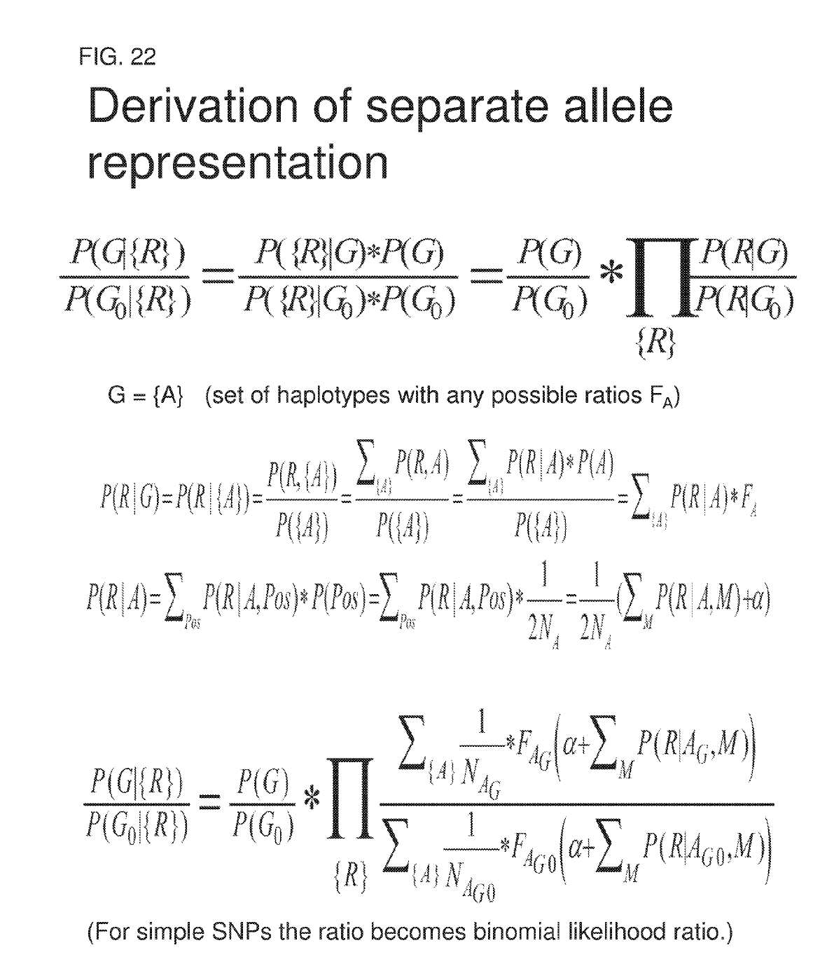

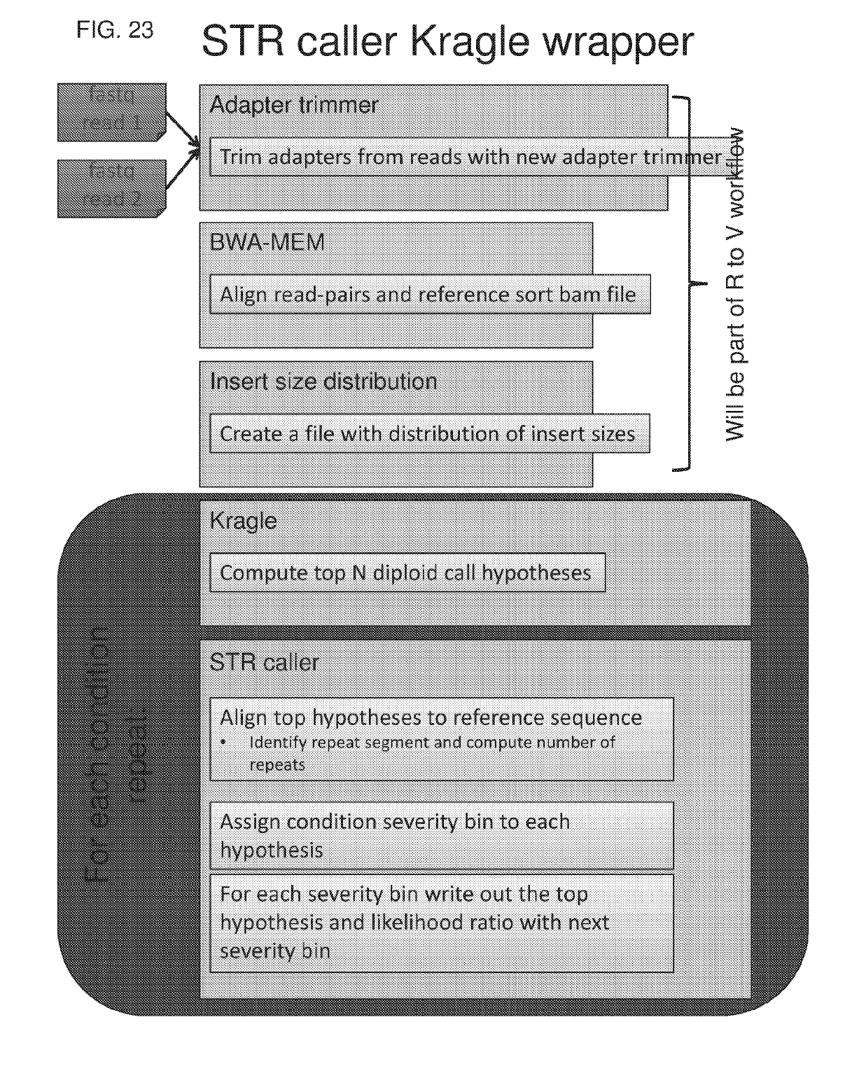

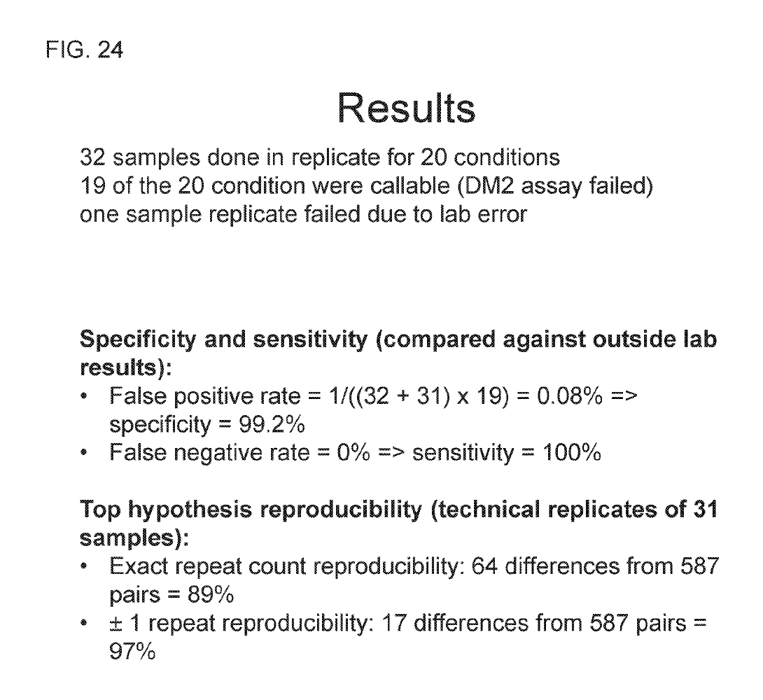

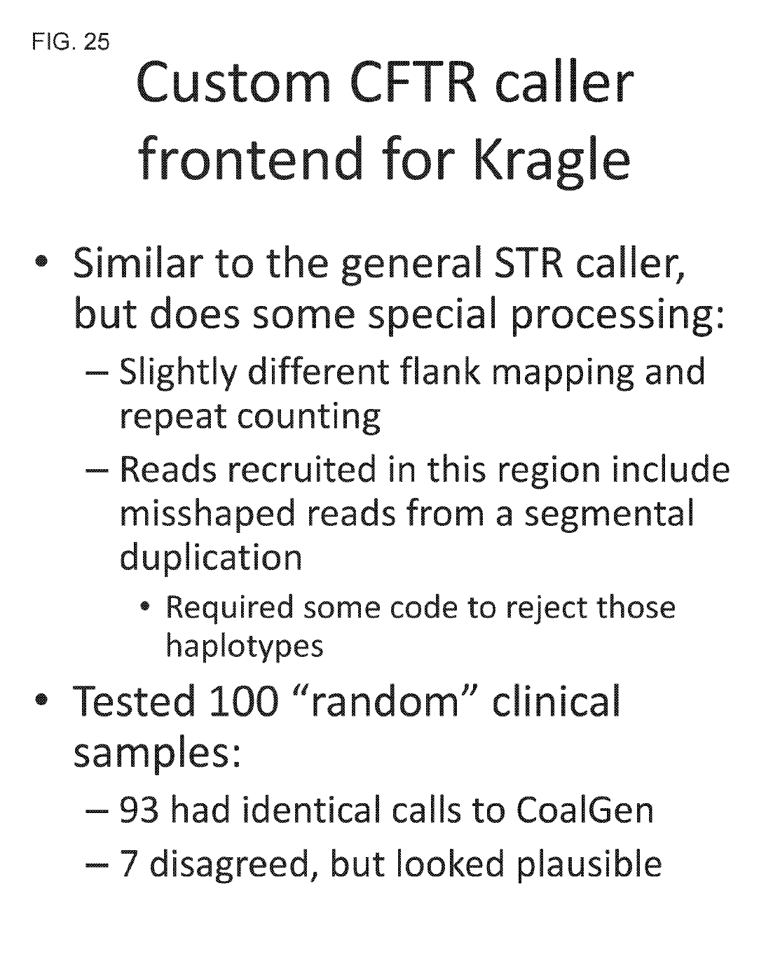

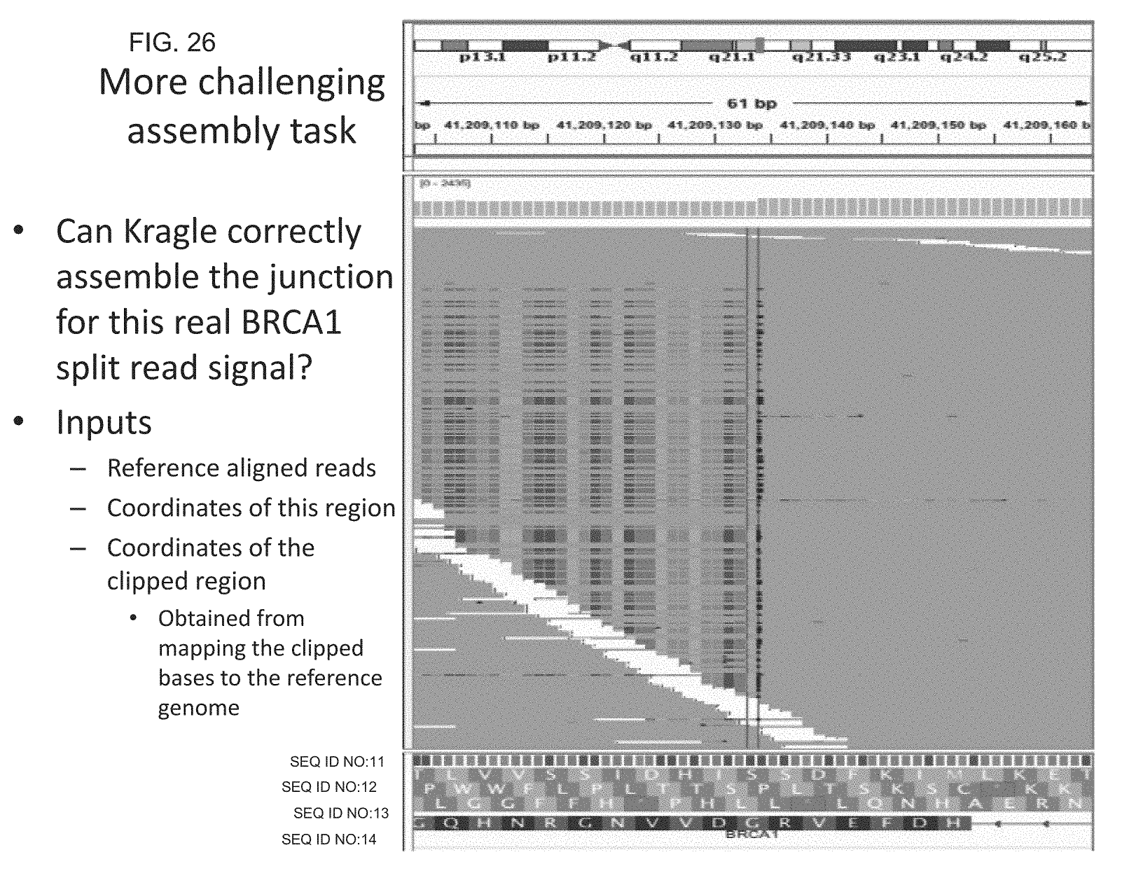

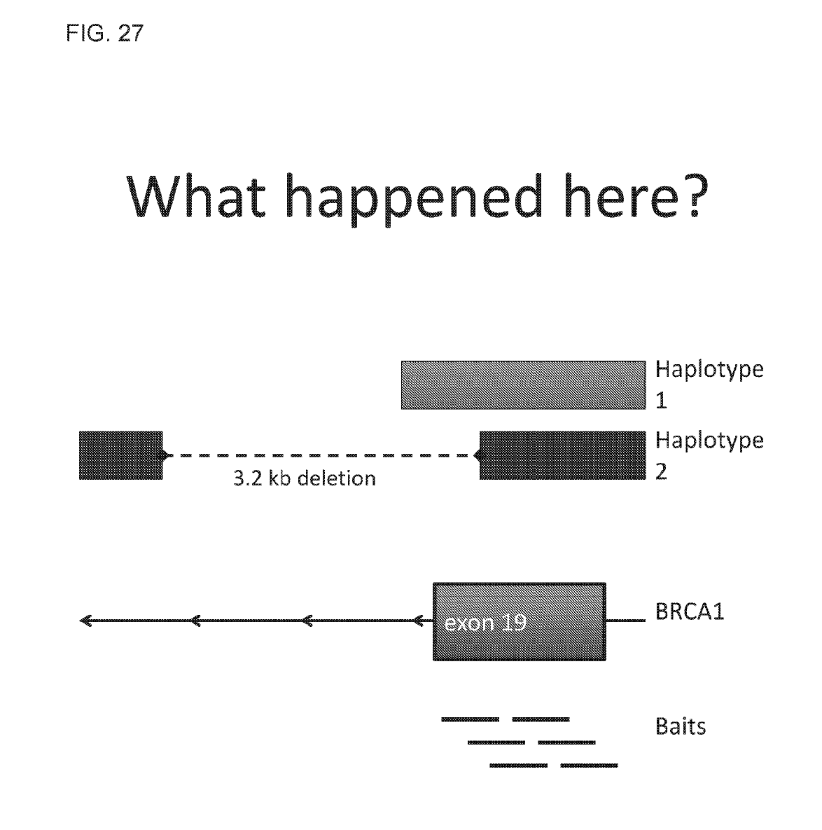

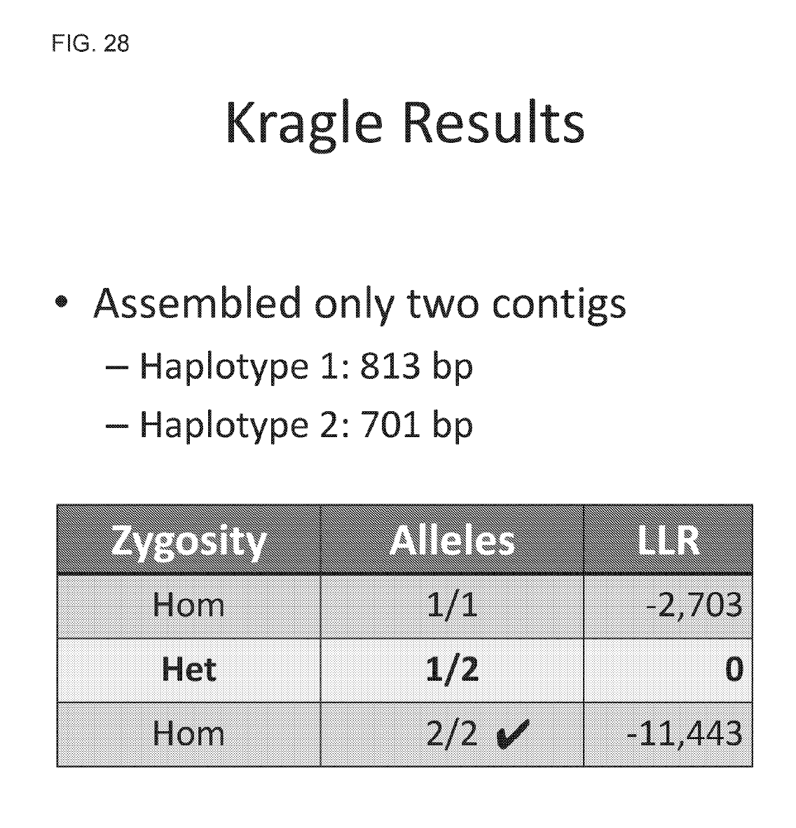

False insertions can be identified by inspecting read mate-pair positions for situations where read-pairs unexpectedly jump over a piece of sequence, where the inserted sequence has read-pairs mapping only within it, but not outside of it (see FIG. 9). The algorithm searches in both forward and reverse directions. It first finds regions where mate-pairs that do not overlap are separated by distance larger than the mean of the insert size distribution. The medians of the middle of the reads mark the beginning and the end of the hypothesized insert. Then the algorithm attempts to locate read-pairs that are fully contained within the hypothesized insert. If such read-pairs are found, the algorithm refines the insert edges as the left-most and the right-most bases of these reads. Then within this interval the algorithm computes at each position the contamination of the fully contained read-pairs by read-pairs that are not fully contained. If at any position the contamination with outside read-pairs drops below a cutoff then this region is reported as a possible false insertion.

Caller

The haplotypes produced by the haplotyper can be filtered for false junctions and insertions and then combined to create haploid, diploid or in general any ploidy sequence hypotheses. For diploid sequence hypotheses the caller explored all possible pairs of haplotypes, including homozygous arrangements consisting of two copies of the same haplotype. The diploid hypothesis evaluation process therefore scaled in computational complexity with the square of the number of haplotypes. Therefore, to avoid ad-hoc haplotype and hypothesis filtering, the hypothesis likelihood evaluation must be computationally efficient. The statistical framework described by Carnevali (Carnevali et al. 2012) was considered but failed in certain aspects. For example Carnevali's framework failed to accommodate any allele ratios (for mosaic and cancer genomes). The statistical framework of equation 2 was extended and modified to accommodate any allele ratios (for mosaic and cancer genomes). This new framework (e.g., see Eq. 1) also allowed precomputation of many components of the likelihood calculation for each haplotype and therefore pairing haplotypes to diploid hypotheses and computing their likelihoods required just a fairly trivial amount of compute.

$$\frac{P ( G \vert \{ R \} )}{P ( {G}_{0} \vert \{ R \} )} = {( \frac{{N}_{{G}_{0}}}{{N}_{G}} )}^{NR} {\prod }_{\{ R \}} \frac{{\sum }_{\{ M \}} P ( R \vert G , M )}{{\sum }_{\{ M \}} P ( R \vert {G}_{0} , M )} \qquad Eq . 2$$

Where G denotes any ploidy genome sequence and G₀ denotes a reference genome sequence, or any other fixed sequence hypothesis. {R} denotes the set of read pairs and the number of read pairs was N_{R}. N_{G} and N_{G0} denote the number of bases in genome G and G₀, respectively. M signifies the mapping locations of the two reads in a read pair and the set of all possible mapping locations of the read pair R was denoted by {M}.

The P(R/G,M) can be computed as a product of the match and mismatch base probabilities given the mapping positions in the genome (Carnevali et al. 2012) and the probability of the insert size implied by the mapping M of the two reads in the read-pair. The match and mismatch probabilities can be deduced from the basecall error rates assigned by the sequencing platform.

P(R|G,M)=P(I_{M})*ΠᵢP(bᵢ|G[M(i)]) Eq. 3

Where I_{M} is the insert size implied by mapping M of the read-pair, and the probability P(I_{M}) can be obtained from an empirical insert-size distribution. The product Πᵢ is taken over all positions i in the read-pair, and the P(bᵢ|G[M(i)]) is a probability that a mapped reference base in genome G at the mapped position i generated the mapped base b in the read-pair. These can be computed using estimated basecall error probabilities provided by the sequencer.

P(bᵢ|G[M(i)])=(1−εᵢ)δ[bᵢ,G[M(i)]]+(εᵢ/3)(1−δ[bᵢ,G[M(i)]]) Eq. 4

In this equation δ is the Kronecker symbol defined to be 1 if its two arguments are identical and 0 otherwise, and εᵢ is the error probability for basecall at position i in the read-pair. G[M(i)] is the base in genome G to which the base bᵢ in the read-pair is mapped using mapping M.

The sum of the read probabilities P(R|G,M) over the entire set of possible mappings can be computationally intractable even for small genomes. The combinations of all possible locations of the two reads can be too many to enumerate. However, there are only few mappings to the genome that will give a significant contribution to the sum. Therefore the sum over all possible mappings can be split into a sum over “good” mappings, called mapping weight W(R,G), and the remaining small contributions to the sum can be aggregated to a small term α:

$${\sum }_{\{ M \}} P ( R \vert G , M ) = {\sum }_{\{ {M}_{good} \}} P ( R \vert G , M ) + α = W ( R , G ) + α \qquad Eq . 5$$

In local de-novo sequencing it is possible to use the α to capture the sum probability of mapping weights outside the assembled region. Some reads, especially reads containing low complexity sequence, can have several mappings to the reference genome, and therefore their placement to the region of interest is uncertain and should be accompanied with a larger α, which effectively reduces their contribution to the likelihood ratio.

The above Eq. 2 can thus be expanded and modified to Eq. 1 below which accommodate alleles with different ratios:

$$\frac{P ( G \vert \{ R \} )}{P ( {G}_{0} \vert \{ R \} )} = {\prod }_{\{ R \}} \frac{{\sum }_{\{ {A}_{G} \}} \frac{1}{{N}_{{A}_{G}}} {F}_{{A}_{G}} ( W ( R , {A}_{G} ) + α )}{{\sum }_{\{ {{A}_{G}}_{0} \}} \frac{1}{{N}_{{A}_{{G}_{0}}}} {F}_{{A}_{{G}_{0}}} ( W ( R , {A}_{{G}_{0}} ) + α )} \qquad Eq . 1$$

where G is a genotype sequence for a predetermined ploidy, G₀ is a reference sequence, {R} is a set of the read mate pairs R, N_{AG} is a number of alleles A_{G} in the genotype sequence _{G}, N_{AG0} is a number of alleles A_{G0} in the reference sequence G₀, and F_{AG} is a fraction of the alleles A_{G} in the genotype sequence G, F_{AG0} is a fraction of the alleles A_{G0} in the reference sequence G₀, W is a read-pair mapping weight, and a is a mapping probability constant. In some embodiments a genotype likelihood ratio is determined according to derivation of equation 1 or variation of equation 1. In diploid genomes the two allele fractions will be 0.5 each.

Using equations above the W(R,A_{G}) can be expressed as Eq. 6 below:

$$W ( R , {A}_{G} ) = {\sum }_{\{ {M}_{good} \}} ( P ( {I}_{M} ) * {\prod }_{i ∈ M} ( ( 1 - {ɛ}_{i} ) δ [ {b}_{i} , G [ M ( i ) ] ] + ( {ɛ}_{i} \text{/} 3 ) ( 1 - δ [ {b}_{i} , G [ M ( i ) ] ] ) ) )$$

As mentioned above the a captures the sum of mapping weights of possible mappings outside of the set of mapping locations M_{good} in the region being assembled. Every mapping program provides a mapping quality value (mapQ), which is the phred-transformed probability of an alignment being wrong (mapQ?=−10*log₁₀(P_{wrongAlignment})). This probability is not the sum of mapping weights at all possible alternative mapping locations, so a scaling was developed that approximates the translation of mapQ to that sum of mapping weights (W_{mapQ}). The scaling aims to make the W_{mapQ} contribution negligibly small for high mapQ values (i.e. 60 in BWA), and have contribution 1 (large value) for mapQ=0. Therefore the a in equation 5 can be further expanded to:

a=W_{mapQ}+α_{R} Eq. 7

Where α_{R} is a very small constant capturing a residual mapping weights to outside locations for very high mapQ values (when W_{mapQ} becomes negligible).

The W_{mapQ} is obtained using the equations below:

W_{mapQ}=10^{mapQ*f/-10} Eq. 8

$$\begin{matrix}f = 1 IF mapQ \leq 30 ELSE ( 1 + \frac{mapQ - 30}{30} * ( \frac{pmax}{60} - 1 ) ) & Eq . 9 \\ pmax = - 10 * {log}_{10} ( {α}_{R} ) + 100 & Eq . 10\end{matrix}$$

Since the W_{mapQ} value is computed for a read-pair, the mapQ value in the above equations is the max value of the two reads in the read-pair. Therefore if one of the reads in the read-pair has a convincing unique mapping in the assembly region, then it is assumes that the second read belongs uniquely to the assembly region as well.

It is evident from the equations above that the operating range of the mapQ transformation is between mapQ values of 30 and 60. For values less than 30 (low quality reads) the W_{mapQ} value becomes comparable to mapping weights of good mappings. This effectively decreases the contribution of the read-pair to the overall probability ratio in equation 1. On the other hand for mapQ values approaching 60 (high quality mappings) the W_{mapQ} becomes smaller than α_{R}, therefore negligible and α≈α_{R}.

The above equations 6-10 allows precomputation of the read pair weights for each read-pair and each allele (supercontig) and can recall the values during the hypothesis likelihood computation. This pre-computation significantly speeds up the computation of the likelihood ratios, and therefore allowed evaluation, in real time, of all diploid hypotheses constructed from thousands of supercontigs. In some embodiments, to facilitate the computation of the read weights for all reads, the caller realigns all reads to all supercontigs.

The likelihood ratio calculation requires the caller to either map all the reads to the reference genome or pick one of the sequence hypotheses as reference. By default the first hypothesis becomes the reference and then all likelihoods are computed relative to the first hypothesis. The log-likelihood ratios of any two hypotheses can be subtracted to get their relative likelihood ratio. This allows computation of the likelihood ratio of the top hypothesis to any other hypothesis to obtain a confidence measure.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

### REFERENCES

Carnevali, P., et al. 2012. Computational Techniques for Human Genome Resequencing Using Mated Gapped Reads. J. Comput. Biol. 19, 279-292.
Idury R M, Waterman M S (1995) J. Comput. Biol. 2(2):291-306.
Pevzner P A, Tang H, Waterman M S (2001) Proc. Natl. Acad. Sci. USA. 98(17):9748-53).
Myers E W (2005) Bioinformatics 21:Suppl 2: ii79-85).
Myers E W, et al., (2000) Science 287(5461):2196-204).
Smith T F, Waterman M S (1981) J. Theor. Biol. 91(2):379-80.
*Smith T F, Waterman M S (1981) J. Mol. Biol. 147(1):195-7.
Needleman S B, Wunsch C D (1970) J. Mol. Biol. 48(3):443-53).

1.-189. (canceled)

190. A computer-implemented method for determining the presence or absence of a genetic alteration in a subject, comprising:
(a) obtaining a set of paired-end sequence reads comprising a plurality of read mate pairs, each pair comprising two read mates, wherein at least one of the two read mates of each pair is mapped to at least one portion of a reference genome comprising a pre-selected genomic region of interest and wherein some of the paired-end sequence reads are not mapped to the at least one portion of the reference genome;
(b) determining a pile-up relationship for the set of sequence reads, wherein the pile-up relationship comprises a plurality of overlaps between two or more reads of the set;
(c) constructing one or more contigs according to the pile-up relationship determined in (b), comprising iteratively adding at least one nucleotide to a position 3′ or 5′ of one or more starter reads wherein the at least one nucleotide added is a majority consensus nucleotide determined according to the plurality of overlaps;
(d) assembling one or more supercontigs, according to the one or more contigs constructed in (c) and/or one or more read mate pairs that bridge two or more of the contigs constructed in (c);
(e) generating a genotype likelihood ratio according to the one or more supercontigs; and
(f) determining the presence or absence of genetic alteration according to the genotype likelihood ratio generated in (e).

191. The method of claim 190, wherein each of the plurality of overlaps is selected according to (i) a first read of the set that comprises a first overlap with a second read of the set, (ii) the first overlap includes an alignment score that is greater than a predetermined alignment score threshold, (iii) the second read extends one or more nucleotides past a 3′ end or a 5′ end of the first read, and (iv) the first overlap includes a highest alignment score of all possible overlaps between the first and second read that satisfies (i), (ii) and (iii).

192. The method of claim 190, wherein the position comprises two different majority consensus nucleotides, constructing the contig comprises generating a copy of the contig, thereby providing two identical intermediate contigs, and adding one of the two different majority consensus nucleotides to each of the two identical intermediate contigs, wherein a different nucleotide is added to each of the two identical intermediate contigs; or
wherein the position comprises three different majority consensus nucleotides, constructing the contig comprises generating two copies of the intermediate contig, thereby providing three identical intermediate contigs, adding one of the three different majority consensus nucleotides to each of the three identical intermediate contigs, wherein a different nucleotide is added to each of the three identical intermediate contigs; or
wherein the position comprises four different majority consensus nucleotides, constructing the contig comprises generating three copies of the intermediate contig, thereby providing four identical intermediate contigs, adding one of the four different majority consensus nucleotides to each of the four identical intermediate contigs, wherein a different nucleotide is added to each of the four identical intermediate contigs.

193. The method of claim 190, wherein the one or more supercontigs comprise a contig that spans a full length of the genomic region of interest; or wherein the one or more supercontigs span a full length of the genomic region of interest.

194. The method of claim 190, wherein the sequence reads are obtained from a sample obtained from a human subject.

195. The method of claim 190, wherein the genotype hypothesis likelihood ratio is determined according to one or more mapping weights.

196. The method of claim 190, wherein the majority consensus nucleotide is determined according to at least 5 reads that are aligned.

197. The method of claim 190, comprising generating a tiling graph according to the pile-up relationship.

198. The method of claim 190, wherein each of the plurality of overlaps is determined according to a k-mer hashing strategy.

199. The method of claim 190, wherein the starter read comprises a read located at the most 5′ side of the pre-selected genomic region of interest, or the starter read comprises a read located at the most 3′ side of the pre-selected genomic region of interest.

200. The method of claim 190, wherein a first contig is joined to a second contig according to multiple read mate pairs.

201. The method of claim 190, wherein the genetic variation comprises a short tandem repeat, or one or more single nucleotide polymorphisms.

202. The method of claim 190, wherein the genotype likelihood ratio is determined according to equation 1
$$\frac{P ( G \vert \{ R \} )}{P ( {G}_{0} \vert \{ R \} )} = {\prod }_{\{ R \}} \frac{{\sum }_{\{ {A}_{G} \}} \frac{1}{{N}_{{A}_{G}}} {F}_{{A}_{G}} ( W ( R , {A}_{G} ) + α )}{{\sum }_{\{ {{A}_{G}}_{0} \}} \frac{1}{{N}_{{A}_{{G}_{0}}}} {F}_{{A}_{{G}_{0}}} ( W ( R , {A}_{{G}_{0}} ) + α )} \qquad Eq . 1$$
where G is a genotype sequence for a predetermined ploidy, G₀ is a reference sequence, {R} is a set of the read mate pairs R, N_{AG} is a number of alleles A_{G} in the genotype sequence _{G}, N_{AG0} is a number of alleles _{AG0} in the reference sequence G₀ and F_{AG} is a fraction of the alleles _{AG} in the genotype sequence G, F_{AG0} is a fraction of the alleles _{AG0} in the reference sequence G₀, W is a read-pair mapping weight, and a is a mapping probability constant.

203. The method of claim 190, wherein the genetic variation is comprised within a gene selected from AR, ATXN1, ATXN2, ATXN7, ATXN8, ATXN10, DMPK, FXN, JPH3, CACNA1A, PPP2R2B, TBP, ATN1, ARX, PHOX2B, PABPN1, ATT, CFTR and BRACA1.

204. The method of claim 190, wherein generating the genotype likelihood ratio of (e) comprises re-aligning the sequence reads to the one or more supercontigs.

205. The method of claim 190, wherein the sequence reads are obtained from a diploid human subject.

206. The method of claim 190, wherein each of the plurality of read mate pairs is not used more than once for the construction of any one of the one or more contigs constructed in (c).

207. The method of claim 190, wherein generating the genotype likelihood ratio comprises determining one or more probable genotypes according to one or more haplotypes, wherein each haplotype is determined according to a supercontig that spans the full length of the genomic region of interest.

208. A non-transitory computer-readable storage medium with an executable program stored thereon, which program is configured to instruct a microprocessor to carry out the method of claim 190.
